SEQUENCE LISTING (271 sequences)

SEQ ID NO: 1 - protein, 737 aa, Artificial sequence
  Synthetic construct
MSDQDHSMDEMTAVVKIEKGVGGNNGGNGNGGGAFSQARSSSTGSSSSTGGGGQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 2 - protein, 975 aa, Artificial sequence
  Synthetic construct
MSGIGNKRAAGEPGTSMPPEKKAAVEDSGTTVETIKLGGVSSTEELDIRTLQTKNRKLAEMLDQRQAIEDELREHIEKLERRQATDDASLLIVNRYWSQFDENIRIILKRYDLEQGLGDLLTERKALVVPEPEPDSDSNQERKDDRERGEGQEPAFSFLATLASSSSEEMESQLQERVESSRRAVSQIVTVYDKLQEKVELLSRKLNSGDNLIVEEAVQELNSFLAQENMRLQELTDLLQEKHRTMSQEFSKLQSKVETAESRVSVLESMIDDLQWDIDKIRKREQRLNRHLAEVLERVNSKGYKVYGAGSSLYGGTITINARKFEEMNAELEENKELAQNRLCELEKLRQDFEEVTTQNEKLKVELRSAVEQVVKETPEYRCMQSQFSVLYNESLQLKAHLDEARTLLHGTRGTHQHQVELIERDEVSLHKKLRTEVIQLEDTLAQVRKEYEMLRIEFEQTLAANEQAGPINREMRHLISSLQNHNHQLKGEVLRYKRKLREAQSDLNKTRLRSGSALLQSQSSTEDPKDEPAELKPDSEDLSSQSSASKASQEDANEIKSKRDEEERERERREKEREREREREKEKEREREKQKLKESEKERDSAKDKEKGKHDDGRKKEAEIIKQLKIELKKAQESQKEMKLLLDMYRSAPKEQRDKVQLMAAEKKSKAELEDLRQRLKDLEDKEKKENKKMADEDALRKIRAVEEQIEYLQKKLAMAKQEEEALLSEMDVTGQAFEDMQEQNIRLMQQLREKDDANFKLMSERIKSNQIHKLLKEEKEELADQVLTLKTQVDAQLQVVRKLEEKEHLLQSNIGTGEKELGLRTQALEMNKRKAMEAAQLADDLKAQLELAQKKLHDFQDEIVENSVTKEKDMFNFKRAQEDISRLRRKLETTKKPDNVPKCDEILMEEIKDYKARLTCPCCNMRKKDAVLTKCFHVFCFECVKTRYDTRQRKCPKCNAAFGANDFHRIYIG

SEQ ID NO: 3 - protein, 1460 aa, Artificial sequence
  Synthetic construct
MAEEQQQPPPQQPDAHQQLPPSAPNSGVALPALVPGLPGTEASALQHKIKNSICKTVQSKVDCILQEVEKFTDLEKLYLYLQLPSGLSNGEKSDQNAMSSSRAQQMHAFSWIRNTLEEHPETSLPKQEVYDEYKSYCDNLGYHPLSAADFGKIMKNVFPNMKARRLGTRGKSKYCYSGLRKKAFVHMPTLPNLDFHKTGDGLEGAEPSGQLQNIDEEVISSACRLVCEWAQKVLSQPFDTVLELARFLVKSHYIGTKSMAALTVMAAAPAGMKGITQPSAFIPTAESNSFQPQVKTLPSPIDAKQQLQRKIQKKQQEQKLQSPLPGESAAKKSESATSNGVTNLPNGNPSILSPQPIGIVVAAVPSPIPVQRTRQLVTSPSPMSSSDGKVLPLNVQVVTQHMQSVKQAPKTPQNVPASPGGDRSARHRYPQILPKPANTSALTIRSPTTVLFTSSPIKTAVVPASHMSSLNVVKMTTISLTPSNSNTPLKHSASVSSATGTTEESRSVPQIKNGSVVSLQSPGSRSSSAGGTSAVEVKVEPETSSDEHPVQCQENSDEAKAPQTPSALLGQKSNTDGALQKPSNEGVIEIKATKVCDQRTKCKSRCNEMLPGTSTGNNQSTITLSVASQNLTFTSSSSPPNGDSINKDPKLCTKSPRKRLSSTLQETQVPPVKKPIVEQLSAATIEGQKQGSVKKDQKVPHSGKTEGSTAGAQIPSKVSVNVSSHIGANQPLNSSALVISDSALEQQTTPSSSPDIKVKLEGSVFLLDSDSKSVGSFNPNGWQQITKDSEFISASCEQQQDISVMTIPEHSDINDLEKSVWELEGMPQDTYSQQLHSQIQESSLNQIQAHSSDQLPLQSELKEFEPSVSQTNESYFPFDDELTQDSIVEELVLMEQQMSMNNSHSYGNCLGMTLQSQSVTPGAPMSSHTSSTHFYHPIHSNGTPIHTPTPTPTPTPTPTPTPTPTSEMIAGSQSLSRESPCSRLAQTTPVDSALGSSRHTPIGTPHSNCSSSVPPSPVECRNPFAFTPISSSMAYHDASIVSSSPVKPMQRPMATHPDKTKLEWMNNGYSGVGNSSVSGHGILPSYQELVEDRFRKPHAFAVPGQSYQSQSRHHDTNFGRLTPVSPVQHQGATVNNTNKQEGFAVPAPLDNKGTNSSASSNFRCRSVSPAVHRQRNLSGSTLYPVSNIPRSNVTPFGSPVTPEVHVFTNVHTDACANNIAQRSQSVPLTVMMQTAFPNALQKQANSKKITNVLLSKLDSDNDDAVRGLGMNNLPSNYTARMNLTQILEPSTVFPSANPQNMIDSSTSVYEFQTPSYLTKSNSTGQINFSPGDNQAQSEIGEQQLDFNSTVKDLLSGDSLQTNQQLVGQGASDLTNTASDFSSDIRLSSELSGSINDLNTLDPNLLFDPGRQQGQDDEATLEELKNDPLFQQICSESMNSMTSSGFEWIESKDHPTVEMLG

SEQ ID NO: 4 - protein, 508 aa, Artificial sequence
  Synthetic construct
MLPTQAGAAAALGRGSALGGSLNRTPTGRPGGGGGTRGANGGRVPGNGAGLGPGRLEREAAAAAATTPAPTAGALYSGSEGDSESGEEEELGAERRGLKRSLSEMEIGMVVGGPEASAAATGGYGPVSGAVSGAKPGKKTRGRVKIKMEFIDNKLRRYTTFSKRKTGIMKKAYELSTLTGTQVLLLVASETGHVYTFATRKLQPMITSETGKALIQTCLNSPDSPPRSDPTTDQRMSATGFEETDLTYQVSESDSSGETKDTLKPAFTVTNLPGTTSTIQTAPSTSTTMQVSSGPSFPITNYLAPVSASVSPSAVSSANGTVLKSTGSGPVSSGGLMQLPTSFTLMPGGAVAQQVPVQAIQVHQAPQQASPSRDSSTDLTQTSSSGTVTLPATIMTSSVPTTVGGHMMYPSPHAVMYAPTSGLGDGSLTVLNAFSQAPSTMQVSHSQVQEPGGVPQVFLTASSGTVQIPVSAVQLHQMAVIGQQAGSSSNLTELQVVNLDTAHSTKSE

SEQ ID NO: 5 - protein, 891 aa, Artificial sequence
  Synthetic construct
MVAPVLETSHVFCCPNRVRGVLNWSSGPRGLLAFGTSCSVVLYDPLKRVVVTNLNGHTARVNCIQWICKQDGSPSTELVSGGSDNQVIHWEIEDNQLLKAVHLQGHEGPVYAVHAVYQRRTSDPALCTLIVSAAADSAVRLWSKKGPEVMCLQTLNFGNGFALALCLSFLPNTDVTWKTGQVERGRAWKPPASLALCSRSCDSMVSCYASILCKALWKEKLHTFWHHNRISFLPSAFRPIPILACGNDDCRIHIFAQQNDQFQKVLSLCGHEDWIRGVEWAAFGRDLFLASCSQDCLIRIWKLYIKSTSLETQDDDNIRLKENTFTIENESVKIAFAVTLETVLAGHENWVNAVHWQPVFYKDGVLQQPVRLLSASMDKTMILWAPDEESGVWLEQVRVGEVGGNTLGFYDCQFNEDGSMIIAHAFHGALHLWKQNTVNPREWTPEIVISGHFDGVQDLVWDPEGEFIITVGTDQTTRLFAPWKRKDQSQVTWHEIARPQIHGYDLKCLAMINRFQFVSGADEKVLRVFSAPRNFVENFCAITGQSLNHVLCNQDSDLPEGATVPALGLSNKAVFQGDIASQPSDEEELLTSTGFEYQQVAFQPSILTEPPTEDHLLQNTLWPEVQKLYGHGYEIFCVTCNSSKTLLASACKAAKKEHAAIILWNTTSWKQVQNLVFHSLTVTQMAFSPNEKFLLAVSRDRTWSLWKKQDTISPEFEPVFSLFAFTNKITSVHSRIIWSCDWSPDSKYFFTGSRDKKVVVWGECDSTDDCIEHNIGPCSSVLDVGGAVTAVSVCPVLHPSQRYVVAVGLECGKICLYTWKKTDQVPEINDWTHCVETSQSQSHTLAIRKLCWKNCSGKTEQKEAEGAEWLHFASCGEDHTVKIHRVNKCAL

SEQ ID NO: 6 - protein, 2427 aa, Artificial sequence
  Synthetic construct
MPLKTRTALSDDPDSSTSTLGNMLELPGTSSSSTSQELPFCQPKKKSTPLKYEVGDLIWAKFKRRPWWPCRICSDPLINTHSKMKVSNRRPYRQYYVEAFGDPSERAWVAGKAIVMFEGRHQFEELPVLRRRGKQKEKGYRHKVPQKILSKWEASVGLAEQYDVPKGSKNRKCIPGSIKLDSEEDMPFEDCTNDPESEHDLLLNGCLKSLAFDSEHSADEKEKPCAKSRARKSSDNPKRTSVKKGHIQFEAHKDERRGKIPENLGLNFISGDISDTQASNELSRIANSLTGSNTAPGSFLFSSCGKNTAKKEFETSNGDSLLGLPEGALISKCSREKNKPQRSLVCGSKVKLCYIGAGDEEKRSDSISICTTSDDGSSDLDPIEHSSESDNSVLEIPDAFDRTENMLSMQKNEKIKYSRFAATNTRVKAKQKPLISNSHTDHLMGCTKSAEPGTETSQVNLSDLKASTLVHKPQSDFTNDALSPKFNLSSSISSENSLIKGGAANQALLHSKSKQPKFRSIKCKHKENPVMAEPPVINEECSLKCCSSDTKGSPLASISKSGKVDGLKLLNNMHEKTRDSSDIETAVVKHVLSELKELSYRSLGEDVSDSGTSKPSKPLLFSSASSQNHIPIEPDYKFSTLLMMLKDMHDSKTKEQRLMTAQNLVSYRSPGRGDCSTNSPVGVSKVLVSGGSTHNSEKKGDGTQNSANPSPSGGDSALSGELSASLPGLLSDKRDLPASGKSRSDCVTRRNCGRSKPSSKLRDAFSAQMVKNTVNRKALKTERKRKLNQLPSVTLDAVLQGDRERGGSLRGGAEDPSKEDPLQIMGHLTSEDGDHFSDVHFDSKVKQSDPGKISEKGLSFENGKGPELDSVMNSENDELNGVNQVVPKKRWQRLNQRRTKPRKRMNRFKEKENSECAFRVLLPSDPVQEGRDEFPEHRTPSASILEEPLTEQNHADCLDSAGPRLNVCDKSSASIGDMEKEPGIPSLTPQAELPEPAVRSEKKRLRKPSKWLLEYTEEYDQIFAPKKKQKKVQEQVHKVSSRCEEESLLARGRSSAQNKQVDENSLISTKEEPPVLEREAPFLEGPLAQSELGGGHAELPQLTLSVPVAPEVSPRPALESEELLVKTPGNYESKRQRKPTKKLLESNDLDPGFMPKKGDLGLSKKCYEAGHLENGITESCATSYSKDFGGGTTKIFDKPRKRKRQRHAAAKMQCKKVKNDDSSKEIPGSEGELMPHRTATSPKETVEEGVEHDPGMPASKKMQGERGGGAALKENVCQNCEKLGELLLCEAQCCGAFHLECLGLTEMPRGKFICNECRTGIHTCFVCKQSGEDVKRCLLPLCGKFYHEECVQKYPPTVMQNKGFRCSLHICITCHAANPANVSASKGRLMRCVRCPVAYHANDFCLAAGSKILASNSIICPNHFTPRRGCRNHEHVNVSWCFVCSEGGSLLCCDSCPAAFHRECLNIDIPEGNWYCNDCKAGKKPHYREIVWVKVGRYRWWPAEICHPRAVPSNIDKMRHDVGEFPVLFFGSNDYLWTHQARVFPYMEGDVSSKDKMGKGVDGTYKKALQEAAARFEELKAQKELRQLQEDRKNDKKPPPYKHIKVNRPIGRVQIFTADLSEIPRCNCKATDENPCGIDSECINRMLLYECHPTVCPAGGRCQNQCFSKRQYPEVEIFRTLQRGWGLRTKTDIKKGEFVNEYVGELIDEEECRARIRYAQEHDITNFYMLTLDKDRIIDAGPKGNYARFMNHCCQPNCETQKWSVNGDTRVGLFALSDIKAGTELTFNYNLECLGNGKTVCKCGAPNCSGFLGVRPKNQPIATEEKSKKFKKKQQGKRRTQGEITKEREDECFSCGDAGQLVSCKKPGCPKVYHADCLNLTKRPAGKWECPWHQCDICGKEAASFCEMCPSSFCKQHREGMLFISKLDGRLSCTEHDPCGPNPLEPGEIREYVPPPVPLPPGPSTHLAEQSTGMAAQAPKMSDKPPADTNQMLSLSKKALAGTCQRPLLPERPLERTDSRPQPLDKVRDLAGSGTKSQSLVSSQRPLDRPPAVAGPRPQLSDKPSPVTSPSSSPSVRSQPLERPLGTADPRLDKSIGAASPRPQSLEKTSVPTGLRLPPPDRLLITSSPKPQTSDRPTDKPHASLSQRLPPPEKVLSAVVQTLVAKEKALRPVDQNTQSKNRAALVMDLIDLTPRQKERAASPHQVTPQADEKMPVLESSSWPASKGLGHMPRAVEKGCVSDPLQTSGKAAAPSEDPWQAVKSLTQARLLSQPPAKAFLYEPTTQASGRASAGAEQTPGPLSQSPGLVKQAKQMVGGQQLPALAAKSGQSFRSLGKAPASLPTEEKKLVTTEQSPWALGKASSRAGLWPIVAGQTLAQSCWSAGSTQTLAQTCWSLGRGQDPKPEQNTLPALNQAPSSHKCAESEQK

SEQ ID NO: 7 - protein, 403 aa, Artificial sequence
  Synthetic construct
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLWCGCIAALTLRADSALVLHFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 8 - protein, 355 aa, Artificial sequence
  Synthetic construct
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 9 - protein, 725 aa, Artificial sequence
  Synthetic construct
MEGDAVEAIVEESETFIKGKERKTYQRRREGGQEEDACHLPQNQTDGGEVVQDVNSSVQMVMMEQLDPTLLQMKTEVMEGTVAPEAEAAVDDTQIITLQVVNMEEQPINIGELQLVQVPVPVTVPVATTSVEELQGAYENEVSKEGLAESEPMICHTLPLPEGFQVVKVGANGEVETLEQGELPPQEDPSWQKDPDYQPPAKKTKKTKKSKLRYTEEGKDVDVSVYDFEEEQQEGLLSEVNAEKVVGNMKPPKPTKIKKKGVKKTFQCELCSYTCPRRSNLDRHMKSHTDERPHKCHLCGRAFRTVTLLRNHLNTHTGTRPHKCPDCDMAFVTSGELVRHRRYKHTHEKPFKCSMCDYASVEVSKLKRHIRSHTGERPFQCSLCSYASRDTYKLKRHMRTHSGEKPYECYICHARFTQSGTMKMHILQKHTENVAKFHCPHCDTVIARKSDLGVHLRKQHSYIEQGKKCRYCDAVFHERYALIQHQKSHKNEKRFKCDQCDYACRQERHMIMHKRTHTGEKPYACSHCDKTFRQKQLLDMHFKRYHDPNFVPAAFVCSKCGKTFTRRNTMARHADNCAGPDGVEGENGGETKKSKRGRKRKMRSKKEDSSDSENAEPDLDDNEDEEEPAVEIEPEPEPQPVTPAPPPAKKRRGRPPGRTNQPKQNQPIIQVEDQNTGAIENIIVEVKKEPDAEPAEGEEEEAQPAATDAPNGDLTPEMILSMMDR

SEQ ID NO: 10 - protein, 763 aa, Artificial sequence
  Synthetic construct
MDHLNEATQGKEHSEMSNNVSDPKGPPAKIARLEQNGSPLGRGRLGSTGAKMQGVPLKHSGHLMKTNLRKGTMLPVFCVVEHYENAIEYDCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSSLAKECPLSQSMISSIVNSTYYANVSAAKCQEFGRWYKHFKKTKDMMVEMDSLSELSQQGANHVNFGQQPVPGNTAEQPPSPAQLSHGSQPSVRTPLPNLHPGLVSTPISPQLVNQQLVMAQLLNQQYAVNRLLAQQSLNQQYLNHPPPVSRSMNKPLEQQVSTNTEVSSEIYQWVRDELKRAGISQAVFARVAFNRTQGLLSEILRKEEDPKTASQSLLVNLRAMQNFLQLPEAERDRIYQDERERSLNAASAMGPAPLISTPPSRPPQVKTATIATERNGKPENNTMNINASIYDEIQQEMKRAKVSQALFAKVAATKSQGWLCELLRWKEDPSPENRTLWENLSMIRRFLSLPQPERDAIYEQESNAVHHHGDRPPHIIHVPAEQIQQQQQQQQQQQQQQQAPPPPQPQQQPQTGPRLPPRQPTVASPAESDEENRQKTRPRTKISVEALGILQSFIQDVGLYPDEEAIQTLSAQLDLPKYTIIKFFQNQRYYLKHHGKLKDNSGLEVDVAEYKEEELLKDLEESVQDKNTNTLFSVKLEEELSVEGNTDINTDLKD

SEQ ID NO: 11 - protein, 396 aa, Artificial sequence
  Synthetic construct
MPNPRPGKPSAPSLALGPSPGASPSWRAAPKASDLLGARGPGGTFQGRDLRGGAHASSSSLNPMPPSQLQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 12 - protein, 525 aa, Artificial sequence
  Synthetic construct
MVSRPEPEGEAMDAELAVAPPGCSHLGSFKVDNWKQNLRAIYQCFVWSGTAEARKRKAKSCICHVCGVHLNRLHSCLYCVFFGCFTKKHIHEHAKAKRHNLAIDLMYGGIYCFLCQDYIYDKDMEIIAKEEQRKAWKMQGVGEKFSTWEPTKRELELLKHNPKRRKITSNCTIGLRGLINLGNTCFMNCIVQALTHTPLLRDFFLSDRHRCEMQSPSSCLVCEMSSLFQEFYSGHRSPHIPYKLLHLVWTHARHLAGYEQQDAHEFLIAALDVLHRHCKGDDNGKKANNPNHCNCIIDQIFTGGLQSDVTCQVCHGVSTTIDPFWDISLDLPGSSTPFWPLSPGSEGNVVNGESHVSGTTTLTDCLRRFTRPEHLGSSAKIKCSGCHSYQESTKQLTMKKLPIVACFHLKRFEHSAKLRRKITTYVSFPLELDMTPFMASSKESRMNGQYQQPTDSLNNDNKYSLFAVVNHQGTLESGHYTSFIRQHKDQWFKCDDAIITKASIKDVLDSEGYLLFYHKQFLEYE

SEQ ID NO: 13 - protein, 187 aa, Artificial sequence
  Synthetic construct
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEARRQQDPSPGSNLGGGDDLKLR

SEQ ID NO: 14 - protein, 453 aa, Artificial sequence
  Synthetic construct
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 15 - protein, 453 aa, Artificial sequence
  Synthetic construct
MDFFRVVENQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 16 - protein, 395 aa, Artificial sequence
  Synthetic construct
MLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 17 - protein, 157 aa, Artificial sequence
  Synthetic construct
MSTPPLAASGMAPGPFAGPQAQQAAREVNTASLCRIGQETVQDIVYRTMEIFQLLRNMQLPNGVTYHTGTYQDRLTKLQDNLRQLSVLFRKLRLVYDKCNENCGGMDPIPVEQLIPYVEEDGSKNDDRAGPPRFASEERREIAEVNKALSSVPEFLP

SEQ ID NO: 18 - protein, 354 aa, Artificial sequence
  Synthetic construct
MKMEEMSLSGLDNSKLEAIAQEIYADLVEDSCLGFCFEVHRAVKCGYFFLDDTDPDSMKDFEIVDQPGLDIFGQVFNQWKSKECVCPNCSRSIAASRFAPHLEKCLGMGRNSSRIANRRIANSNNMNKSESDQEDNDDINDNDWSYGSEKKAKKRKSDKLWYLPFQNPNSPRRSKSLKHKNGELSNSDPFKYNNSTGISYETLGPEELRSLLTTQCGVISEHTKKMCTRSLRCPQHTDEQRRTVRIYFLGPSAVLPEVESSLDNDSFDMTDSQALISRLQWDGSSDLSPSDSGSSKTSENQGWGLGTNSSESRKTKKKKSHLSLVGTASGLGSNKKKKPKPPAPPTPSIYDDIN

SEQ ID NO: 19 - protein, 2177 aa, Artificial sequence
  Synthetic construct
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 20 - protein, 440 aa, Artificial sequence
  Synthetic construct
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 21 - protein, 859 aa, Artificial sequence
  Synthetic construct
MKIGSGFLSGGGGTGSSGGSGSGGGGSGGGGGGGSSGRRAEMEPTFPQAPAAEPPPPPAPDMTFKKEPAASAAAFPSQRTSWGFLQSLVSIKQEKPADPEEQQSHHHHHHHHYGGLFAGAEERSPGLGGGEGGSHGVIQDLSILHQHVQQQPAQHHRDVLLSSSSRTDDHHGTEEPKQDTNVKKAKRPKPESQGIKAKRKPSASSKPSLVGDGEGAILSPSQKPHICDHCSAAFRSSYHLRRHVLIHTGERPFQCSQCSMGFIQKYLLQRHEKIHSREKPFGCDQCSMKFIQKYHMERHKRTHSGEKPYKCDTCQQYFSRTDRLLKHRRTCGEVIVKGATSAEPGSSNHTNMGNLAVLSQGNTSSSRRKTKSKSIAIENKEQKTGKTNESQISNNINMQSYSVEMPTVSSSGGIIGTGIDELQKRVPKLIFKKGSRKNTDKNYLNFVSPLPDIVGQKSLSGKPSGSLGIVSNNSVETIGLLQSTSGKQGQISSNYDDAMQFSKKRRYLPTASSNSAFSINVGHMVSQQSVIQSAGVSVLDNEAPLSLIDSSALNAEIKSCHDKSGIPDEVLQSILDQYSNKSESQKEDPFNIAEPRVDLHTSGEHSELVQEENLSPGTQTPSNDKASMLQEYSKYLQQAFEKSTNASFTLGHGFQFVSLSSPLHNHTLFPEKQIYTTSPLECGFGQSVTSVLPSSLPKPPFGMLFGSQPGLYLSALDATHQQLTPSQELDDLIDSQKNLETSSAFQSSSQKLTSQKEQKNLESSTGFQIPSQELASQIDPQKDIEPRTTYQIENFAQAFGSQFKSGSRVPMTFITNSNGEVDHRVRTSVSDFSGYTNMMSDVSEPCSTRVKTPTSQSYR

SEQ ID NO: 22 - protein, 589 aa, Artificial sequence
  Synthetic construct
MKRVRTEQIQMAVSCYLKRRQYVDSDGPLKQGLRLSQTAEEMAANLTVQSESGCANIVSAAPCQAEPQQYEVQFGRLRNFLTDSDSQHSHEVMPLLYPLFVYLHLNLVQNSPKSTVESFYSRFHGMFLQNASQKDVIEQLQTTQTIQDILSNFKLRAFLDNKYVVRLQEDSYNYLIRYLQSDNNTALCKVLTLHIHLDVQPAKRTDYQLYASGSSSRSENNGLEPPDMPSPILQNEAALEVLQESIKRVKDGPPSLTTICFYAFYNTEQLLNTAEISPDSKLLAAGFDNSCIKLWSLRSKKLKSEPHQVDVSRIHLACDILEEEDDEDDNAGTEMKILRGHCGPVYSTRFLADSSGLLSCSEDMSIRYWDLGSFTNTVLYQGHAYPVWDLDISPYSLYFASGSHDRTARLWSFDRTYPLRIYAGHLADVDCVKFHPNSNYLATGSTDKTVRLWSAQQGNSVRLFTGHRGPVLSLAFSPNGKYLASAGEDQRLKLWDLASGTLYKELRGHTDNITSLTFSPDSGLIASASMDNSVRVWDIRNTYCSAPADGSSSELVGVYTGQMSNVLSVQFMACNLLLVTGITQENQEH

SEQ ID NO: 23 - protein, 169 aa, Artificial sequence
  Synthetic construct
MAAESLPFSFGTLSSWELEAWYEDLQEVLSSDENGGTYVSPPGNEEEESKIFTTLDPASLAWLTEEEPEPAEVTSTSQSPHSPDSSQSSLAQEEEEEDQGRTRKRKQSGHSPARAGKQRMKEKEQENERKVAQLAEENERLKQEIERLTREVEATRRALIDRMVNLHQA

SEQ ID NO: 24 - protein, 1186 aa, Artificial sequence
  Synthetic construct
MDISTRSKDPGSAERTAQKRKFPSPPHSSNGHSPQDTSTSPIKKKKKPGLLNSNNKEQSELRHGPFYYMKQPLTTDPVDVVPQDGRNDFYCWVCHREGQVLCCELCPRVYHAKCLRLTSEPEGDWFCPECEKITVAECIETQSKAMTMLTIEQLSYLLKFAIQKMKQPGTDAFQKPVPLEQHPDYAEYIFHPMDLCTLEKNAKKKMYGCTEAFLADAKWILHNCIIYNGGNHKLTQIAKVVIKICEHEMNEIEVCPECYLAACQKRDNWFCEPCSNPHPLVWAKLKGFPFWPAKALRDKDGQVDARFFGQHDRAWVPINNCYLMSKEIPFSVKKTKSIFNSAMQEMEVYVENIRRKFGVFNYSPFRTPYTPNSQYQMLLDPTNPSAGTAKIDKQEKVKLNFDMTASPKILMSKPVLSGGTGRRISLSDMPRSPMSTNSSVHTGSDVEQDAEKKATSSHFSASEESMDFLDKSTASPASTKTGQAGSLSGSPKPFSPQLSAPITTKTDKTSTTGSILNLNLDRSKAEMDLKELSESVQQQSTPVPLISPKRQIRSRFQLNLDKTIESCKAQLGINEISEDVYTAVEHSDSEDSEKSDSSDSEYISDDEQKSKNEPEDTEDKEGCQMDKEPSAVKKKPKPTNPVEIKEELKSTSPASEKADPGAVKDKASPEPEKDFSEKAKPSPHPIKDKLKGKDETDSPTVHLGLDSDSESELVIDLGEDHSGREGRKNKKEPKEPSPKQDVVGKTPPSTTVGSHSPPETPVLTRSSAQTSAAGATATTSTSSTVTVTAPAPAATGSPVKKQRPLLPKETAPAVQRVVWNSSSKFQTSSQKWHMQKMQRQQQQQQQQNQQQQPQSSQGTRYQTRQAVKAVQQKEITQSPSTSTITLVTSTQSSPLVTSSGSMSTLVSSVNADLPIATASADVAADIAKYTSKMMDAIKGTMTEIYNDLSKNTTGSTIAEIRRLRIEIEKLQWLHQQELSEMKHNLELTMAEMRQSLEQERDRLIAEVKKQLELEKQQAVDETKKKQWCANCKKEAIFYCCWNTSYCDYPCQQAHWPEHMKSCTQSATAPQQEADAEVNTETLNKSSQGSSSSTQSAPSETASASKEKETSAEKSKESGSTLDLSGSRETPSSILLGSNQGSDHSRSNKSSWSSSDEKRGSTRSDHNTSTSTKSLLPKESRLDTFWD

SEQ ID NO: 25 - protein, 1454 aa, Artificial sequence
  Synthetic construct
MAPVQLENHQLVPPGGGGGGSGGPPSAPAPPPPGAAVAAAAAAAASPGYRLSTLIEFLLHRAYSELMVLTDLLPRKSDVERKIEIVQFASRTRQLFVRLLALVKWANNAGKVEKCAMISSFLDQQAILFVDTADRLASLARDALVHARLPSFAIPYAIDVLTTGSYPRLPTCIRDKIIPPDPITKIEKQATLHQLNQILRHRLVTTDLPPQLANLTVANGRVKFRVEGEFEATLTVMGDDPDVPWRLLKLEILVEDKETGDGRALVHSMQISFIHQLVQSRLFADEKPLQDMYNCLHSFCLSLQLEVLHSQTLMLIRERWGDLVQVERYHAGKCLSLSVWNQQVLGRKTGTASVHKVTIKIDENDVSKPLQIFHDPPLPASDSKLVERAMKIDHLSIEKLLIDSVHARAHQKLQELKAILRGFNANENSSIETALPALVVPILEPCGNSECLHIFVDLHSGMFQLMLYGLDQATLDDMEKSVNDDMKRIIPWIQQLKFWLGQQRCKQSIKHLPTISSETLQLSNYSTHPIGNLSKNKLFIKLTRLPQYYIVVEMLEVPNKPTQLSYKYYFMSVNAADREDSPAMALLLQQFKENIQDLVFRTKTGKQTRTNAKRKLSDDPCPVESKKTKRAGEMCAFNKVLAHFVAMCDTNMPFVGLRLELSNLEIPHQGVQVEGDGFSHAIRLLKIPPCKGITEETQKALDRSLLDCTFRLQGRNNRTWVAELVFANCPLNGTSTREQGPSRHVYLTYENLLSEPVGGRKVVEMFLNDWNSIARLYECVLEFARSLPDIPAHLNIFSEVRVYNYRKLILCYGTTKGSSISIQWNSIHQKFHISLGTVGPNSGCSNCHNTILHQLQEMFNKTPNVVQLLQVLFDTQAPLNAINKLPTVPMLGLTQRTNTAYQCFSILPQSSTHIRLAFRNMYCIDIYCRSRGVVAIRDGAYSLFDNSKLVEGFYPAPGLKTFLNMFVDSNQDARRRSVNEDDNPPSPIGGDMMDSLISQLQPPPQQQPFPKQPGTSGAYPLTSPPTSYHSTVNQSPSMMHTQSPGNLHAASSPSGALRAPSPASFVPTPPPSSHGISIGPGASFASPHGTLDPSSPYTMVSPSGRAGNWPGSPQVSGPSPAARMPGMSPANPSLHSPVPDASHSPRAGTSSQTMPTNMPPPRKLPQRSWAASIPTILTHSALNILLLPSPTPGLVPGLAGSYLCSPLERFLGSVIMRRHLQRIIQQETLQLINSNEPGVIMFKTDALKCRVALSPKTNQTLQLKVTPENAGQWKPDELQVLEKFFETRVAGPPFKANTLIAFTKLLGAPTHILRDCVHIMKLELFPDQATQLKWNVQFCLTIPPSAPPIAPPGTPAVVLKSKMLFFLQLTQKTSVPPQEPVSIIVPIIYDMASGTTQQADIPRQQNSSVAAPMMVSNILKRFAEMNPPRQGECTIFAAVRDLMANLTLPPGGRP

SEQ ID NO: 26 - protein, 631 aa, Artificial sequence
  Synthetic construct
MFYAHFVLSKRGPLAKIWLAAHWDKKLTKAHVFECNLESSVESIISPKVKMALRTSGHLLLGVVRIYHRKAKYLLADCNEAFIKIKMAFRPGVVDLPEENREAAYNAITLPEEFHDFDQPLPDLDDIDVAQQFSLNQSRVEEITMREEVGNISILQENDFGDFGMDDREIMREGSAFEDDDMLVSTTTSNLLLESEQSTSNLNEKINHLEYEDQYKDDNFGEGNDGGILDDKLISNNDGGIFDDPPALSEAGVMLPEQPAHDDMDEDDNVSMGGPDSPDSVDPVEPMPTMTDQTTLVPNEEEAFALEPIDITVKETKAKRKRKLIVDSVKELDSKTIRAQLSDYSDIVTTLDLAPPTKKLMMWKETGGVEKLFSLPAQPLWNNRLLKLFTRCLTPLVPEDLRKRRKGGEADNLDEFLKEFENPEVPREDQQQQHQQRDVIDEPIIEEPSRLQESVMEASRTNIDESAMPPPPPQGVKRKAGQIDPEPVMPPQQVEQMEIPPVELPPEEPPNICQLIPELELLPEKEKEKEKEKEDDEEEEDEDASGGDQDQEERRWNKRTQQMLHGLQRALAKTGAESISLLELCRNTNRKQAAAKFYSFLVLKKQQAIELTQEEPYSDIIATPGPRFHII

SEQ ID NO: 27 - protein, 467 aa, Artificial sequence
  Synthetic construct
MATGADVRDILELGGPEGDAASGTISKKDIINPDKKKSKKSSETLTFKRPEGMHREVYALLYSDKKDAPPLLPSDTGQGYRTVKAKLGSKKVRPWKWMPFTNPARKDGAMFFHWRRAAEEGKDYPFARFNKTVQVPVYSEQEYQLYLHDDAWTKAETDHLFDLSRRFDLRFVVIHDRYDHQQFKKRSVEDLKERYYHICAKLANVRAVPGTDLKIPVFDAGHERRRKEQLERLYNRTPEQVAEEEYLLQELRKIEARKKEREKRSQDLQKLITAADTTAEQRRTERKAPKKKLPQKKEAEKPAVPETAGIKFPDFKSAGVTLRSQRMKLPSSVGQKKIKALEQMLLELGVELSPTPTEELVHMFNELRSDLVLLYELKQACANCEYELQMLRHRHEALARAGVLGGPATPASGPGPASAEPAVTEPGLGPDPKDTIIDVVGAPLTPNSRKRRESASSSSSVKKAKKP

SEQ ID NO: 28 - protein, 85 aa, Artificial sequence
  Synthetic construct
MATYSLANERLRALEDIEREIGAILQNAGTVILELSKEKTNERLLDRQAAAFTASVQHVEAELSAQIRYLTQLPDGLTNSNSGKK

SEQ ID NO: 29 - protein, 390 aa, Artificial sequence
  Synthetic construct
MALLTPAPGSQSSQFQLMKALLKHESVGSQPLQDRVLQVPVLAHGGCCREDKVVASRLTPESQGLLKVEDVALTLTPEWTQQDSSQGNLCRDEKQENHGSLVSLGDEKQTKSRDLPPAEELPEKEHGKISCHLREDIAQIPTCAEAGEQEGRLQRKQKNATGGRRHICHECGKSFAQSSGLSKHRRIHTGEKPYECEECGKAFIGSSALVIHQRVHTGEKPYECEECGKAFSHSSDLIKHQRTHTGEKPYECDDCGKTFSQSCSLLEHHRIHTGEKPYQCSMCGKAFRRSSHLLRHQRIHTGDKNVQEPEQGEAWKSRMESQLENVETPMSYKCNECERSFTQNTGLIEHQKIHTGEKPYQCNACGKGFTRISYLVQHQRSHVGKNILSQ

SEQ ID NO: 30 - protein, 677 aa, Artificial sequence
  Synthetic construct
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 31 - protein, 787 aa, Artificial sequence
  Synthetic construct
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 32 - DNA, 20 bp, Artificial sequence
  Synthetic construct
caacagattatcacaaatcg

SEQ ID NO: 33 - DNA, 20 bp, Artificial sequence
  Synthetic construct
catcatccggacaccaacag

SEQ ID NO: 34 - DNA, 20 bp, Artificial sequence
  Synthetic construct
gtatgtgaccaatgtaccag

SEQ ID NO: 35 - DNA, 20 bp, Artificial sequence
  Synthetic construct
ttactaccagtggatcatca

SEQ ID NO: 36 - DNA, 20 bp, Artificial sequence
  Synthetic construct
acttcggcaagactttgagg

SEQ ID NO: 37 - DNA, 20 bp, Artificial sequence
  Synthetic construct
gcatcgcaccatgtctcagg

SEQ ID NO: 38 - DNA, 20 bp, Artificial sequence
  Synthetic construct
ggagggcactaccactacgc

SEQ ID NO: 39 - DNA, 20 bp, Artificial sequence
  Synthetic construct
tcggttgacaatcaatagtg

SEQ ID NO: 40 - DNA, 20 bp, Artificial sequence
  Synthetic construct
acaacgataccaataggttg

SEQ ID NO: 41 - DNA, 20 bp, Artificial sequence
  Synthetic construct
agctgaatcactgataacaa

SEQ ID NO: 42 - DNA, 20 bp, Artificial sequence
  Synthetic construct
ctggattcggaataccctag

SEQ ID NO: 43 - DNA, 20 bp, Artificial sequence
  Synthetic construct
gaagcgggctaattccaaga

SEQ ID NO: 44 - DNA, 20 bp, Artificial sequence
  Synthetic construct
aggttggtgactgtgaacgc

SEQ ID NO: 45 - DNA, 20 bp, Artificial sequence
  Synthetic construct
agttcatcgacaacaagctg

SEQ ID NO: 46 - DNA, 20 bp, Artificial sequence
  Synthetic construct
gggctgacactagcagacac

SEQ ID NO: 47 - DNA, 20 bp, Artificial sequence
  Synthetic construct
tctgttgtggggtctgaacg

SEQ ID NO: 48 - DNA, 20 bp, Artificial sequence
  Synthetic construct
aatttcatgccaagtcacct

SEQ ID NO: 49 - DNA, 20 bp, Artificial sequence
  Synthetic construct
ccagtaccaatattagcatg

SEQ ID NO: 50 - DNA, 20 bp, Artificial sequence
  Synthetic construct
gttattgtacaggttcgagt

SEQ ID NO: 51 - DNA, 20 bp, Artificial sequence
  Synthetic construct
tgataatcaagtgattcact

SEQ ID NO: 52 - DNA, 20 bp, Artificial sequence
  Synthetic construct
aagcacataaagatgaacgg

SEQ ID NO: 53 - DNA, 20 bp, Artificial sequence
  Synthetic construct
gaattgctagttaaaacgcc

SEQ ID NO: 54 - DNA, 20 bp, Artificial sequence
  Synthetic construct
gccctatcggcagtactacg

SEQ ID NO: 55 - DNA, 20 bp, Artificial sequence
  Synthetic construct
tatgcatgatagtaagacga

SEQ ID NO: 56 - DNA, 20 bp, Artificial sequence
  Synthetic construct
gagaacctcggaacatacgg

SEQ ID NO: 57 - DNA, 20 bp, Artificial sequence
  Synthetic construct
gcagatcgagtcctacccca

SEQ ID NO: 58 - DNA, 20 bp, Artificial sequence
  Synthetic construct
tcttcttgtctcggcccatg

SEQ ID NO: 59 - DNA, 20 bp, Artificial sequence
  Synthetic construct
tgagaacgcatctcagcccg

SEQ ID NO: 60 - DNA, 20 bp, Artificial sequence
  Synthetic construct
aaaccagggccaccgaaagg

SEQ ID NO: 61 - DNA, 20 bp, Artificial sequence
  Synthetic construct
ccctcgcgcttgaggccgcg

SEQ ID NO: 62 - DNA, 20 bp, Artificial sequence
  Synthetic construct
cttcggtctcttcgacgacg

SEQ ID NO: 63 - DNA, 20 bp, Artificial sequence
  Synthetic construct
tcggggtaatagaacgcagg

SEQ ID NO: 64 - DNA, 20 bp, Artificial sequence
  Synthetic construct
cgatccaaatttgaacgccg

SEQ ID NO: 65 - DNA, 20 bp, Artificial sequence
  Synthetic construct
gagcaaactgcgttatacag

SEQ ID NO: 66 - DNA, 20 bp, Artificial sequence
  Synthetic construct
ttaccccagaaccagacgga

SEQ ID NO: 67 - DNA, 20 bp, Artificial sequence
  Synthetic construct
tttgtgcagttatgccagca

SEQ ID NO: 68 - DNA, 20 bp, Artificial sequence
  Synthetic construct
atgctaagtacctgtgaaag

SEQ ID NO: 69 - DNA, 20 bp, Artificial sequence
  Synthetic construct
cattgaatatgattgcaagg

SEQ ID NO: 70 - DNA, 20 bp, Artificial sequence
  Synthetic construct
taggtgttgatacgagccca

SEQ ID NO: 71 - DNA, 20 bp, Artificial sequence
  Synthetic construct
tattcatagatctactgaca

SEQ ID NO: 72 - DNA, 20 bp, Artificial sequence
  Synthetic construct
acccaggcatcatccgacaa

SEQ ID NO: 73 - DNA, 20 bp, Artificial sequence
  Synthetic construct
cccacccacagggatcaacg

SEQ ID NO: 74 - DNA, 20 bp, Artificial sequence
  Synthetic construct
cctacttaggcactgccagg

SEQ ID NO: 75 - DNA, 20 bp, Artificial sequence
  Synthetic construct
gagggtgccaccatgactag

SEQ ID NO: 76 - DNA, 20 bp, Artificial sequence
  Synthetic construct
acctggtgtggacccacgcg

SEQ ID NO: 77 - DNA, 20 bp, Artificial sequence
  Synthetic construct
cctcgaactgcaccataggt

SEQ ID NO: 78 - DNA, 20 bp, Artificial sequence
  Synthetic construct
gccattgatctgatgtacgg

SEQ ID NO: 79 - DNA, 20 bp, Artificial sequence
  Synthetic construct
tggggctctgcatctcacag

SEQ ID NO: 80 - DNA, 20 bp, Artificial sequence
  Synthetic construct
aagtcgacatactctcggct

SEQ ID NO: 81 - DNA, 20 bp, Artificial sequence
  Synthetic construct
cctgcctcacctcacactcg

SEQ ID NO: 82 - DNA, 20 bp, Artificial sequence
  Synthetic construct
gccgacttacgatttccgag

SEQ ID NO: 83 - DNA, 20 bp, Artificial sequence
  Synthetic construct
ggagtctgtgttatctggaa

SEQ ID NO: 84 - DNA, 20 bp, Artificial sequence
  Synthetic construct
cacttcgaccgacaaacctg

SEQ ID NO: 85 - DNA, 20 bp, Artificial sequence
  Synthetic construct
ctgatcgtaggaccacggtg

SEQ ID NO: 86 - DNA, 20 bp, Artificial sequence
  Synthetic construct
ggcagtggagtggttcaggg

SEQ ID NO: 87 - DNA, 20 bp, Artificial sequence
  Synthetic construct
tagatgatcagaccaagccc

SEQ ID NO: 88 - DNA, 20 bp, Artificial sequence
  Synthetic construct
agagtgcatcgacccctcgg

SEQ ID NO: 89 - DNA, 20 bp, Artificial sequence
  Synthetic construct
ctgcggggaggactccgtcg

SEQ ID NO: 90 - DNA, 20 bp, Artificial sequence
  Synthetic construct
cttcggggagacaacgacgg

SEQ ID NO: 91 - DNA, 20 bp, Artificial sequence
  Synthetic construct
gctgcaccgagtcgtagtcg

SEQ ID NO: 92 - DNA, 20 bp, Artificial sequence
  Synthetic construct
aatcagatgacaatgagtca

SEQ ID NO: 93 - DNA, 20 bp, Artificial sequence
  Synthetic construct
caatacaatatgccacaggg

SEQ ID NO: 94 - DNA, 20 bp, Artificial sequence
  Synthetic construct
cctaaccatatgcctatgca

SEQ ID NO: 95 - DNA, 20 bp, Artificial sequence
  Synthetic construct
ggcatgttgtgagagcgtgg

SEQ ID NO: 96 - DNA, 20 bp, Artificial sequence
  Synthetic construct
acactggaacatatcaagac

SEQ ID NO: 97 - DNA, 20 bp, Artificial sequence
  Synthetic construct
gacaaatgcaatgaaaactg

SEQ ID NO: 98 - DNA, 20 bp, Artificial sequence
  Synthetic construct
ggacatcgtgtaccgcacca

SEQ ID NO: 99 - DNA, 20 bp, Artificial sequence
  Synthetic construct
ggccgcccgggaagtcaaca

SEQ ID NO: 100 - DNA, 20 bp, Artificial sequence
  Synthetic construct
cacggaccctgatagcatga

SEQ ID NO: 101 - DNA, 20 bp, Artificial sequence
  Synthetic construct
catcgctcaggagatatacg

SEQ ID NO: 102 - DNA, 20 bp, Artificial sequence
  Synthetic construct
gcagccgaatcgccaaccgc

SEQ ID NO: 103 - DNA, 20 bp, Artificial sequence
  Synthetic construct
gcttcgcagcctgctaacca

SEQ ID NO: 104 - DNA, 20 bp, Artificial sequence
  Synthetic construct
acatcgactgctggacaatg

SEQ ID NO: 105 - DNA, 20 bp, Artificial sequence
  Synthetic construct
cagtgagtagtgccaaacca

SEQ ID NO: 106 - DNA, 20 bp, Artificial sequence
  Synthetic construct
gtggcgtactgcacgtgtcg

SEQ ID NO: 107 - DNA, 20 bp, Artificial sequence
  Synthetic construct
ttcacattatgaccaacacc

SEQ ID NO: 108 - DNA, 20 bp, Artificial sequence
  Synthetic construct
atgatgtttgatgaccgtcg

SEQ ID NO: 109 - DNA, 20 bp, Artificial sequence
  Synthetic construct
ctgttgggacataccgctcg

SEQ ID NO: 110 - DNA, 20 bp, Artificial sequence
  Synthetic construct
gatgatatgagccctcgtcg

SEQ ID NO: 111 - DNA, 20 bp, Artificial sequence
  Synthetic construct
taaaatcaaagaacttcgag

SEQ ID NO: 112 - DNA, 20 bp, Artificial sequence
  Synthetic construct
cctccactggaagacacggt

SEQ ID NO: 113 - DNA, 20 bp, Artificial sequence
  Synthetic construct
cgaacagccccccatagtgg

SEQ ID NO: 114 - DNA, 20 bp, Artificial sequence
  Synthetic construct
gaggataacacgcattgcgg

SEQ ID NO: 115 - DNA, 20 bp, Artificial sequence
  Synthetic construct
tgctgagtaatacgtcacgg

SEQ ID NO: 116 - DNA, 20 bp, Artificial sequence
  Synthetic construct
cgggacacgtctacttggtg

SEQ ID NO: 117 - DNA, 20 bp, Artificial sequence
  Synthetic construct
gcagaacgaggctgccctag

SEQ ID NO: 118 - DNA, 20 bp, Artificial sequence
  Synthetic construct
gcggaccagtgtacagcacg

SEQ ID NO: 119 - DNA, 20 bp, Artificial sequence
  Synthetic construct
taaggtgaggactttgcaca

SEQ ID NO: 120 - DNA, 20 bp, Artificial sequence
  Synthetic construct
atttccaggaggtgaaacat

SEQ ID NO: 121 - DNA, 20 bp, Artificial sequence
  Synthetic construct
ctggtatgaggacctgcaag

SEQ ID NO: 122 - DNA, 20 bp, Artificial sequence
  Synthetic construct
gactggaatctggagagtga

SEQ ID NO: 123 - DNA, 20 bp, Artificial sequence
  Synthetic construct
tcagccaagccagagaagca

SEQ ID NO: 124 - DNA, 20 bp, Artificial sequence
  Synthetic construct
agatgtattccgcatagtca

SEQ ID NO: 125 - DNA, 20 bp, Artificial sequence
  Synthetic construct
cacttagcgtgataaacccg

SEQ ID NO: 126 - DNA, 20 bp, Artificial sequence
  Synthetic construct
ctcttccgcccaaacttccg

SEQ ID NO: 127 - DNA, 20 bp, Artificial sequence
  Synthetic construct
ggagcgcggcatatccgaca

SEQ ID NO: 128 - DNA, 20 bp, Artificial sequence
  Synthetic construct
atcacacatagcgacgaagt

SEQ ID NO: 129 - DNA, 20 bp, Artificial sequence
  Synthetic construct
cagagcatctctagctaacg

SEQ ID NO: 130 - DNA, 20 bp, Artificial sequence
  Synthetic construct
ctaactctgctacccaagtg

SEQ ID NO: 131 - DNA, 20 bp, Artificial sequence
  Synthetic construct
taatgttaatccgagaacgg

SEQ ID NO: 132 - DNA, 20 bp, Artificial sequence
  Synthetic construct
aagtgttgtttgatcagtca

SEQ ID NO: 133 - DNA, 20 bp, Artificial sequence
  Synthetic construct
acatactctaagtcaggcag

SEQ ID NO: 134 - DNA, 20 bp, Artificial sequence
  Synthetic construct
gtgtaatttagagagcagcg

SEQ ID NO: 135 - DNA, 20 bp, Artificial sequence
  Synthetic construct
tctgttcagactctaatagg

SEQ ID NO: 136 - DNA, 20 bp, Artificial sequence
  Synthetic construct
atgctgggcacgaacgacgg

SEQ ID NO: 137 - DNA, 20 bp, Artificial sequence
  Synthetic construct
catggataacaacaaaacgc

SEQ ID NO: 138 - DNA, 20 bp, Artificial sequence
  Synthetic construct
gaagctaccccagaaaaagg

SEQ ID NO: 139 - DNA, 20 bp, Artificial sequence
  Synthetic construct
ggacattatcaacccggaca

SEQ ID NO: 140 - DNA, 20 bp, Artificial sequence
  Synthetic construct
cacagcaggattcatctcag

SEQ ID NO: 141 - DNA, 20 bp, Artificial sequence
  Synthetic construct
gccgactcagcgcctcgcgg

SEQ ID NO: 142 - DNA, 20 bp, Artificial sequence
  Synthetic construct
gctcaggcctgagtaaacac

SEQ ID NO: 143 - DNA, 20 bp, Artificial sequence
  Synthetic construct
tcaccagcttctgcacatgt

SEQ ID NO: 144 - DNA, 20 bp, Artificial sequence
  Synthetic construct
agaggaggagacacatgtcg

SEQ ID NO: 145 - DNA, 20 bp, Artificial sequence
  Synthetic construct
catacaccatgtccatagag

SEQ ID NO: 146 - DNA, 20 bp, Artificial sequence
  Synthetic construct
gccttctgacaattcagccc

SEQ ID NO: 147 - DNA, 20 bp, Artificial sequence
  Synthetic construct
gttctgtagacttcacatgc

SEQ ID NO: 148 - DNA, 20 bp, Artificial sequence
  Synthetic construct
cagccaggacaacaatgcga

SEQ ID NO: 149 - DNA, 20 bp, Artificial sequence
  Synthetic construct
gtggccttaatgttctcctg

SEQ ID NO: 150 - DNA, 20 bp, Artificial sequence
  Synthetic construct
gttcattgtacaatatatgg

SEQ ID NO: 151 - DNA, 20 bp, Artificial sequence
  Synthetic construct
taagaggtcagaccgtcgtg

SEQ ID NO: 152 - DNA, 30 bp, Artificial sequence
  Synthetic construct
aaaccaacagattatcacaaatcgaggaag

SEQ ID NO: 153 - DNA, 30 bp, Artificial sequence
  Synthetic construct
ccatcatcatccggacaccaacagtggggc

SEQ ID NO: 154 - DNA, 30 bp, Artificial sequence
  Synthetic construct
ctcagtatgtgaccaatgtaccagtggccc

SEQ ID NO: 155 - DNA, 30 bp, Artificial sequence
  Synthetic construct
aactttactaccagtggatcatcagggacc

SEQ ID NO: 156 - DNA, 30 bp, Artificial sequence
  Synthetic construct
agaaacttcggcaagactttgaggaggtca

SEQ ID NO: 157 - DNA, 30 bp, Artificial sequence
  Synthetic construct
aaaagcatcgcaccatgtctcaggaggtac

SEQ ID NO: 158 - DNA, 30 bp, Artificial sequence
  Synthetic construct
tgcaggagggcactaccactacgcaggcgt

SEQ ID NO: 159 - DNA, 30 bp, Artificial sequence
  Synthetic construct
agtatcggttgacaatcaatagtgaggcat

SEQ ID NO: 160 - DNA, 30 bp, Artificial sequence
  Synthetic construct
tgccacaacgataccaataggttgaggaga

SEQ ID NO: 161 - DNA, 30 bp, Artificial sequence
  Synthetic construct
ccaaagctgaatcactgataacaagggcag

SEQ ID NO: 162 - DNA, 30 bp, Artificial sequence
  Synthetic construct
tttcctggattcggaataccctagaggaac

SEQ ID NO: 163 - DNA, 30 bp, Artificial sequence
  Synthetic construct
caaggaagcgggctaattccaagacggtgt

SEQ ID NO: 164 - DNA, 30 bp, Artificial sequence
  Synthetic construct
cggcaggttggtgactgtgaacgccggctt

SEQ ID NO: 165 - DNA, 30 bp, Artificial sequence
  Synthetic construct
atggagttcatcgacaacaagctgcggcgc

SEQ ID NO: 166 - DNA, 30 bp, Artificial sequence
  Synthetic construct
actggggctgacactagcagacactggtgc

SEQ ID NO: 167 - DNA, 30 bp, Artificial sequence
  Synthetic construct
ctggtctgttgtggggtctgaacggggtgg

SEQ ID NO: 168 - DNA, 30 bp, Artificial sequence
  Synthetic construct
ttgcaatttcatgccaagtcacctgggtaa

SEQ ID NO: 169 - DNA, 30 bp, Artificial sequence
  Synthetic construct
tcccccagtaccaatattagcatgtggcaa

SEQ ID NO: 170 - DNA, 30 bp, Artificial sequence
  Synthetic construct
gtctgttattgtacaggttcgagtaggtga

SEQ ID NO: 171 - DNA, 30 bp, Artificial sequence
  Synthetic construct
gatctgataatcaagtgattcactgggaaa

SEQ ID NO: 172 - DNA, 30 bp, Artificial sequence
  Synthetic construct
tttgaagcacataaagatgaacggagggga

SEQ ID NO: 173 - DNA, 30 bp, Artificial sequence
  Synthetic construct
tgaggaattgctagttaaaacgccaggtaa

SEQ ID NO: 174 - DNA, 30 bp, Artificial sequence
  Synthetic construct
ggaggccctatcggcagtactacgtggagg

SEQ ID NO: 175 - DNA, 30 bp, Artificial sequence
  Synthetic construct
aagatatgcatgatagtaagacgaaggagc

SEQ ID NO: 176 - DNA, 30 bp, Artificial sequence
  Synthetic construct
tacagagaacctcggaacatacggaggtag

SEQ ID NO: 177 - DNA, 30 bp, Artificial sequence
  Synthetic construct
gacagcagatcgagtcctaccccacggaca

SEQ ID NO: 178 - DNA, 30 bp, Artificial sequence
  Synthetic construct
gttctcttcttgtctcggcccatgcggttc

SEQ ID NO: 179 - DNA, 30 bp, Artificial sequence
  Synthetic construct
tccatgagaacgcatctcagcccgaggtgc

SEQ ID NO: 180 - DNA, 30 bp, Artificial sequence
  Synthetic construct
gccgaaaccagggccaccgaaaggcggcgg

SEQ ID NO: 181 - DNA, 30 bp, Artificial sequence
  Synthetic construct
ggcgccctcgcgcttgaggccgcgcggtcc

SEQ ID NO: 182 - DNA, 30 bp, Artificial sequence
  Synthetic construct
cagccttcggtctcttcgacgacgcggccg

SEQ ID NO: 183 - DNA, 30 bp, Artificial sequence
  Synthetic construct
gggttcggggtaatagaacgcaggcggcgg

SEQ ID NO: 184 - DNA, 30 bp, Artificial sequence
  Synthetic construct
gtgacgatccaaatttgaacgccgtggaca

SEQ ID NO: 185 - DNA, 30 bp, Artificial sequence
  Synthetic construct
aaaagagcaaactgcgttatacagaggagg

SEQ ID NO: 186 - DNA, 30 bp, Artificial sequence
  Synthetic construct
ccacttaccccagaaccagacggatggggg

SEQ ID NO: 187 - DNA, 30 bp, Artificial sequence
  Synthetic construct
gcagtttgtgcagttatgccagcagggaca

SEQ ID NO: 188 - DNA, 30 bp, Artificial sequence
  Synthetic construct
ttctatgctaagtacctgtgaaagggggca

SEQ ID NO: 189 - DNA, 30 bp, Artificial sequence
  Synthetic construct
acgccattgaatatgattgcaaggaggagc

SEQ ID NO: 190 - DNA, 30 bp, Artificial sequence
  Synthetic construct
ctgataggtgttgatacgagcccagggtgc

SEQ ID NO: 191 - DNA, 30 bp, Artificial sequence
  Synthetic construct
ggcttattcatagatctactgacaggggga

SEQ ID NO: 192 - DNA, 30 bp, Artificial sequence
  Synthetic construct
cctcacccaggcatcatccgacaagggctc

SEQ ID NO: 193 - DNA, 30 bp, Artificial sequence
  Synthetic construct
tgtccccacccacagggatcaacgtggcca

SEQ ID NO: 194 - DNA, 30 bp, Artificial sequence
  Synthetic construct
tctccctacttaggcactgccaggcggacc

SEQ ID NO: 195 - DNA, 30 bp, Artificial sequence
  Synthetic construct
cccggagggtgccaccatgactaggggcag

SEQ ID NO: 196 - DNA, 30 bp, Artificial sequence
  Synthetic construct
ctgcacctggtgtggacccacgcgaggcac

SEQ ID NO: 197 - DNA, 30 bp, Artificial sequence
  Synthetic construct
atcacctcgaactgcaccataggtgggtgg

SEQ ID NO: 198 - DNA, 30 bp, Artificial sequence
  Synthetic construct
ctcagccattgatctgatgtacggaggcat

SEQ ID NO: 199 - DNA, 30 bp, Artificial sequence
  Synthetic construct
gagctggggctctgcatctcacagcggtgc

SEQ ID NO: 200 - DNA, 30 bp, Artificial sequence
  Synthetic construct
ttctaagtcgacatactctcggctaggtgt

SEQ ID NO: 201 - DNA, 30 bp, Artificial sequence
  Synthetic construct
cccgcctgcctcacctcacactcgcggctc

SEQ ID NO: 202 - DNA, 30 bp, Artificial sequence
  Synthetic construct
gccagccgacttacgatttccgagcggccg

SEQ ID NO: 203 - DNA, 30 bp, Artificial sequence
  Synthetic construct
gaatggagtctgtgttatctggaaaggctg

SEQ ID NO: 204 - DNA, 30 bp, Artificial sequence
  Synthetic construct
cttccacttcgaccgacaaacctgaggtca

SEQ ID NO: 205 - DNA, 30 bp, Artificial sequence
  Synthetic construct
aggactgatcgtaggaccacggtggggatg

SEQ ID NO: 206 - DNA, 30 bp, Artificial sequence
  Synthetic construct
taaaggcagtggagtggttcagggaggcac

SEQ ID NO: 207 - DNA, 30 bp, Artificial sequence
  Synthetic construct
aaactagatgatcagaccaagcccgggagc

SEQ ID NO: 208 - DNA, 30 bp, Artificial sequence
  Synthetic construct
cctcagagtgcatcgacccctcggtggtct

SEQ ID NO: 209 - DNA, 30 bp, Artificial sequence
  Synthetic construct
tgccctgcggggaggactccgtcgaggaga

SEQ ID NO: 210 - DNA, 30 bp, Artificial sequence
  Synthetic construct
ctcccttcggggagacaacgacggcggtgg

SEQ ID NO: 211 - DNA, 30 bp, Artificial sequence
  Synthetic construct
tacggctgcaccgagtcgtagtcgaggtca

SEQ ID NO: 212 - DNA, 30 bp, Artificial sequence
  Synthetic construct
acagaatcagatgacaatgagtcagggaca

SEQ ID NO: 213 - DNA, 30 bp, Artificial sequence
  Synthetic construct
tcagcaatacaatatgccacagggaggcgg

SEQ ID NO: 214 - DNA, 30 bp, Artificial sequence
  Synthetic construct
agggcctaaccatatgcctatgcagggacc

SEQ ID NO: 215 - DNA, 30 bp, Artificial sequence
  Synthetic construct
tgaaggcatgttgtgagagcgtggaggtgg

SEQ ID NO: 216 - DNA, 30 bp, Artificial sequence
  Synthetic construct
taccacactggaacatatcaagaccggtta

SEQ ID NO: 217 - DNA, 30 bp, Artificial sequence
  Synthetic construct
atatgacaaatgcaatgaaaactgtggtgg

SEQ ID NO: 218 - DNA, 30 bp, Artificial sequence
  Synthetic construct
tgcaggacatcgtgtaccgcaccatggaga

SEQ ID NO: 219 - DNA, 30 bp, Artificial sequence
  Synthetic construct
agcaggccgcccgggaagtcaacacggcgt

SEQ ID NO: 220 - DNA, 30 bp, Artificial sequence
  Synthetic construct
acgacacggaccctgatagcatgaaggatt

SEQ ID NO: 221 - DNA, 30 bp, Artificial sequence
  Synthetic construct
aggccatcgctcaggagatatacgcggacc

SEQ ID NO: 222 - DNA, 30 bp, Artificial sequence
  Synthetic construct
aacagcagccgaatcgccaaccgccggtga

SEQ ID NO: 223 - DNA, 30 bp, Artificial sequence
  Synthetic construct
aggagcttcgcagcctgctaaccacggtga

SEQ ID NO: 224 - DNA, 30 bp, Artificial sequence
  Synthetic construct
atccacatcgactgctggacaatgaggatg

SEQ ID NO: 225 - DNA, 30 bp, Artificial sequence
  Synthetic construct
cagtcagtgagtagtgccaaaccaaggcac

SEQ ID NO: 226 - DNA, 30 bp, Artificial sequence
  Synthetic construct
atgggtggcgtactgcacgtgtcgtggctg

SEQ ID NO: 227 - DNA, 30 bp, Artificial sequence
  Synthetic construct
acctttcacattatgaccaacaccaggtca

SEQ ID NO: 228 - DNA, 30 bp, Artificial sequence
  Synthetic construct
tacaatgatgtttgatgaccgtcgcggacg

SEQ ID NO: 229 - DNA, 30 bp, Artificial sequence
  Synthetic construct
taaactgttgggacataccgctcggggcca

SEQ ID NO: 230 - DNA, 30 bp, Artificial sequence
  Synthetic construct
ttatgatgatatgagccctcgtcgaggacc

SEQ ID NO: 231 - DNA, 30 bp, Artificial sequence
  Synthetic construct
gtgctaaaatcaaagaacttcgagaggtaa

SEQ ID NO: 232 - DNA, 30 bp, Artificial sequence
  Synthetic construct
tatgcctccactggaagacacggtaggcat

SEQ ID NO: 233 - DNA, 30 bp, Artificial sequence
  Synthetic construct
ccagcgaacagccccccatagtggtggtgg

SEQ ID NO: 234 - DNA, 30 bp, Artificial sequence
  Synthetic construct
agaggaggataacacgcattgcgggggagg

SEQ ID NO: 235 - DNA, 30 bp, Artificial sequence
  Synthetic construct
ctgctgctgagtaatacgtcacggtggtgc

SEQ ID NO: 236 - DNA, 30 bp, Artificial sequence
  Synthetic construct
gatgcgggacacgtctacttggtggggctc

SEQ ID NO: 237 - DNA, 30 bp, Artificial sequence
  Synthetic construct
ttctgcagaacgaggctgccctagaggtct

SEQ ID NO: 238 - DNA, 30 bp, Artificial sequence
  Synthetic construct
cactgcggaccagtgtacagcacgaggttc

SEQ ID NO: 239 - DNA, 30 bp, Artificial sequence
  Synthetic construct
tatgtaaggtgaggactttgcacagggcag

SEQ ID NO: 240 - DNA, 30 bp, Artificial sequence
  Synthetic construct
cttcatttccaggaggtgaaacataggtac

SEQ ID NO: 241 - DNA, 30 bp, Artificial sequence
  Synthetic construct
aagcctggtatgaggacctgcaagaggtcc

SEQ ID NO: 242 - DNA, 30 bp, Artificial sequence
  Synthetic construct
ctctgactggaatctggagagtgagggctc

SEQ ID NO: 243 - DNA, 30 bp, Artificial sequence
  Synthetic construct
tcagtcagccaagccagagaagcagggtca

SEQ ID NO: 244 - DNA, 30 bp, Artificial sequence
  Synthetic construct
tggaagatgtattccgcatagtcagggtgc

SEQ ID NO: 245 - DNA, 30 bp, Artificial sequence
  Synthetic construct
cagacacttagcgtgataaacccggggaca

SEQ ID NO: 246 - DNA, 30 bp, Artificial sequence
  Synthetic construct
cccgctcttccgcccaaacttccgcggctg

SEQ ID NO: 247 - DNA, 30 bp, Artificial sequence
  Synthetic construct
tgggggagcgcggcatatccgacaaggaaa

SEQ ID NO: 248 - DNA, 30 bp, Artificial sequence
  Synthetic construct
ttgtatcacacatagcgacgaagtgggcta

SEQ ID NO: 249 - DNA, 30 bp, Artificial sequence
  Synthetic construct
ggaccagagcatctctagctaacgaggcca

SEQ ID NO: 250 - DNA, 30 bp, Artificial sequence
  Synthetic construct
aacactaactctgctacccaagtgcggtta

SEQ ID NO: 251 - DNA, 30 bp, Artificial sequence
  Synthetic construct
actctaatgttaatccgagaacggtgggga

SEQ ID NO: 252 - DNA, 30 bp, Artificial sequence
  Synthetic construct
gaacaagtgttgtttgatcagtcatggttg

SEQ ID NO: 253 - DNA, 30 bp, Artificial sequence
  Synthetic construct
agacacatactctaagtcaggcagtggctg

SEQ ID NO: 254 - DNA, 30 bp, Artificial sequence
  Synthetic construct
tcgagtgtaatttagagagcagcgtggaga

SEQ ID NO: 255 - DNA, 30 bp, Artificial sequence
  Synthetic construct
gtgctctgttcagactctaataggaggtta

SEQ ID NO: 256 - DNA, 30 bp, Artificial sequence
  Synthetic construct
tttgatgctgggcacgaacgacggcggaag

SEQ ID NO: 257 - DNA, 30 bp, Artificial sequence
  Synthetic construct
cggtcatggataacaacaaaacgcaggtca

SEQ ID NO: 258 - DNA, 30 bp, Artificial sequence
  Synthetic construct
aaaagaagctaccccagaaaaaggaggctg

SEQ ID NO: 259 - DNA, 30 bp, Artificial sequence
  Synthetic construct
agaaggacattatcaacccggacaaggtag

SEQ ID NO: 260 - DNA, 30 bp, Artificial sequence
  Synthetic construct
tggacacagcaggattcatctcaggggaat

SEQ ID NO: 261 - DNA, 30 bp, Artificial sequence
  Synthetic construct
cggagccgactcagcgcctcgcgggggcct

SEQ ID NO: 262 - DNA, 30 bp, Artificial sequence
  Synthetic construct
caaagctcaggcctgagtaaacacaggaga

SEQ ID NO: 263 - DNA, 30 bp, Artificial sequence
  Synthetic construct
ctgttcaccagcttctgcacatgtaggaat

SEQ ID NO: 264 - DNA, 30 bp, Artificial sequence
  Synthetic construct
gtgcagaggaggagacacatgtcgtggtca

SEQ ID NO: 265 - DNA, 30 bp, Artificial sequence
  Synthetic construct
cttgcatacaccatgtccatagagaggatg

SEQ ID NO: 266 - DNA, 30 bp, Artificial sequence
  Synthetic construct
caaggccttctgacaattcagcccgggcag

SEQ ID NO: 267 - DNA, 30 bp, Artificial sequence
  Synthetic construct
ttgggttctgtagacttcacatgcaggtgg

SEQ ID NO: 268 - DNA, 30 bp, Artificial sequence
  Synthetic construct
atggcagccaggacaacaatgcgacggcca

SEQ ID NO: 269 - DNA, 30 bp, Artificial sequence
  Synthetic construct
ctgggtggccttaatgttctcctgtggatt

SEQ ID NO: 270 - DNA, 30 bp, Artificial sequence
  Synthetic construct
ctctgttcattgtacaatatatggcggatg

SEQ ID NO: 271 - DNA, 30 bp, Artificial sequence
  Synthetic construct
gaattaagaggtcagaccgtcgtggggcag

(19) United States
(12) Patent Application Publication
(10) Pub. No.: US 2021/0340496 A1
(43) Pub. Date: Nov. 4, 2021

(54) COMPOSITIONS AND METHODS FOR MODIFYING REGULATORY T CELLS

(71) Applicant: The Regents of the University of California, Oakland, CA (US)

(72) Inventors: Alexander Marson, Oakland, CA (US); Jessica T. Cortez, Oakland, CA (US); Jeffrey A. Bluestone, Oakland, CA (US); Eric Shifrut, Oakland, CA (US); Frederic Van Gool, Oakland, CA (US)

(73) Assignee: The Regents of the University of California, Oakland, CA (US)

(21) Appl. No.: 17/284,396

(22) PCT Filed: Oct. 10, 2019
(86) PCT No.: PCT/US2019/055674

§ 371 Date: Apr. 9, 2021

Related U.S. Application Data
(60) Provisional application No. 62/744,058, filed on Oct. 10, 2018.

(51) Int. Cl. C12N 5/0783; C12N 15/90; A61K 35/17; C12N 15/11; C12N 9/22

(52) U.S. Cl. CPC C12N 5/0637; C12N 15/907; A61K 35/17; C12N 2800/80; C12N 9/22; C12N 2310/20; C12N 15/11

(57) ABSTRACT

Provided herein are compositions and methods for modifying regulatory T cells. The inventors have identified nuclear factors that influence expression of Foxp3, a key transcriptional regulator of Treg cells. Treg cells can be modified by inhibiting and/or overexpressing one or more of these nuclear factors to produce stabilized Treg cells or destabilized Treg cells.

### PRIOR RELATED APPLICATIONS

This application claims the benefit of U.S. Provisional Application No. 62/744,058, filed on Oct. 10, 2018, which is hereby incorporated by reference in its entirety.

### BACKGROUND OF THE INVENTION

Regulatory T cells (Treg cells) play a role in regulating the immune response. In some cases, for example, in some cancers, Treg cells inhibit the ability of the immune system to target and destroy cancer cells. In other cases, for example in autoimmune diseases, Treg cells are unavailable to control the immune system. Methods to stabilize Treg cells for the treatment of autoimmune diseases or actively destabilize Treg cells to ablate tolerogenic effects in a tumor microenvironment have great therapeutic potential.

### SUMMARY OF THE INVENTION

The present invention is directed to compositions and methods for modifying Treg cells. The inventors have identified nuclear factors that influence expression of Foxp3, a key transcriptional regulator of Treg cells. Treg cells can be modified by inhibiting and/or overexpressing one or more of these nuclear factors to produce stabilized Treg cells or destabilized Treg cells. In some examples, stabilized Treg cells are used to treat autoimmune disorders, assist in organ transplantation, to treat graft versus host disease, or inflammation. Examples of autoimmune diseases include but are not limited to: type 1 diabetes, rheumatoid arthritis, inflammatory bowel disease, multiple sclerosis, and multi-organ autoimmune syndromes. In other examples, destabilized Treg cells are used to treat cancer. For example, in some embodiments, destabilized Tregs can be used to target solid tumors, e.g., where Treg cells contribute to a immunosuppressive microenvironment. Examples of such cancers include but are not limited to ovarian cancer.

Provided herein is a method of increasing human regulatory T (Treg) cell stability, the method comprising: inhibiting expression of a nuclear factor set forth in Table 1 and/or overexpressing a nuclear factor set forth in Table 2 in the human Treg cell.

Also provided is a method of decreasing human Treg cell stability is provided, the method comprising: inhibiting expression of a nuclear factor set forth in Table 2 and/or overexpressing a nuclear factor set forth in Table 1 in the human Treg cell.

In some embodiments, the inhibiting comprises reducing expression of a nuclear factor, or reducing expression of a polynucleotide encoding the nuclear factor in a Treg cell. In some embodiments, the overexpressing comprises increasing expression of a nuclear factor, or increasing expression of a polynucleotide encoding the nuclear factor in a Treg cell.

In some embodiments, the inhibiting in a Treg cell comprises contacting a polynucleotide encoding the protein with a targeted nuclease, a guide RNA (gRNA), an siRNA, an antisense RNA, microRNA (miRNA), or short hairpin RNA (shRNA). In some embodiments, the inhibiting comprises contacting the polynucleotide encoding the nuclear factor with at least one gRNA and optionally a targeted nuclease, wherein the at least one gRNA comprises a sequence selected from Table 3. In some embodiments, the inhibiting comprises mutating the polynucleotide encoding the protein. In some embodiments, the inhibiting comprises contacting the polynucleotide with a targeted nuclease.

In some embodiments, the targeted nuclease introduces a double-stranded break in a target region in the polynucleotide. In some embodiments, the targeted nuclease is an RNA-guided nuclease. In some embodiments, the RNA-guided nuclease is a Cpf1 nuclease or a Cas9 nuclease and the method further comprises introducing into a Treg cell a gRNA that specifically hybridizes to a target region in the polynucleotide. In some embodiments, the Cpf1 nuclease or the Cas9 nuclease and the gRNA are introduced into the Treg cell as a ribonucleoprotein (RNP) complex. In some embodiments, the inhibiting comprises performing clustered regularly interspaced short palindromic repeats (CRISPR)/Cas genome editing.

In some embodiments, the Treg cell is administered to a human following the inhibiting and/or the overexpressing. In some embodiments, the Treg cell is obtained from a human prior to treating the Treg cell to inhibit expression of the nuclear factor and/or overexpress the nuclear factor, and the treated Treg cell is reintroduced into a human. In some embodiments, expression of a nuclear factor is inhibited and/or a nuclear factor is overexpressed in an in vivo Treg cell. In some embodiments, the human has an autoimmune disorder, GVHD, inflammation, or is an organ transplantation recipient. In some embodiments, the human has cancer.

In another embodiment, provided herein is a Treg cell made by any of the methods described herein. In another embodiment, the present invention provides a Treg cell comprising a genetic modification or heterologous polynucleotide that inhibits expression of a nuclear factor selected set forth in Table 1 and/or a heterologous polynucleotide that encodes a protein encoded by a nuclear factor set forth in Table 2. In another embodiment, the present invention provides a Treg cell comprising a genetic modification or heterologous polynucleotide that inhibits expression of a nuclear factor set forth in Table 2 and/or a heterologous polynucleotide that encodes a polypeptide encoded by a nuclear factor set forth in Table 1.

In another embodiment, provided herein is a Treg comprising at least one guide RNA (gRNA) comprising a sequence selected from Table 3. In some embodiments, the expression of a nuclear factor set forth in Table 1 or Table 2 is reduced in the Treg cell relative to the expression of the nuclear factor in a Treg cell not comprising a gRNA.

In another embodiment, provided herein is a method of destabilizing Tregs in a subject in need thereof, comprising inhibiting expression of a one or more nuclear factors set forth in Table 2 and/or overexpressing one or more nuclear factors set forth in Table 1, in the humanTreg cells of the subject. In some embodiments, the Treg cells are destabilized in vivo. In other embodiments, the Treg cells are destabilized ex vivo. In some embodiments, the subject has cancer.

In another embodiment, provided herein is a method of stabilizing Tregs in a subject in need thereof, comprising inhibiting expression of a one or more nuclear factors set forth in Table 1 and/or overexpressing one or more nuclear factors set forth in Table 2, in the humanTreg cells of the subject. In some embodiments, the Treg cells are stabilized in vivo. In other embodiments, the Treg cells are stabilized ex vivo. In some embodiments, the subject has an autoimmune disorder.

In another embodiment, provided herein is a method of treating an autoimmune disorder in a subject, the method comprising administering a population of stabilized Treg cells to a subject that has an autoimmune disease. In another embodiment, the present invention provides a method of treating cancer in a subject, the method comprising administering a population of destabilized Treg cells to a subject that has cancer.

In another embodiment, provided herein is a method of treating an autoimmune disorder, GVHD, or inflammation, or assisting in organ transplantation treatment in a subject, the method comprising: (a) obtaining Treg cells from the subject (e.g., that has an autoimmune disorder); (b) modifying the Treg cells by inhibiting expression of a nuclear factor set forth in Table 1 and/or overexpressing a nuclear factor set forth in Table 2 in the Treg cells; and (c) administering the modified Treg cells to the subject.

In another embodiment, the present invention provides a method of treating cancer in a subject, the method comprising: (a) obtaining Treg cells from a subject that has cancer; (b) modifying the Treg cells by inhibiting expression of a nuclear factor set forth in Table 2 and/or overexpressing a nuclear factor set forth in Table 1 in the Treg cells; and (c) administering the modified Treg cells to the subject.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is schematic of the Treg Fate Reporter Mouse that was used to identify Foxp3+T regs and Foxp3-ex Tregs upon inhibition of nuclear factors in a CRISPR screen.

FIG. 2a is a schematic of the pooled CRISPR screening strategy that was used to identify nuclear factors that affect Foxp3 stability.

FIG. 2b is a volcano plot for hits from the screen. The X-axis shows a Z-score for gene-level log 2 fold-change (LFC); median of LFC for all single guide RNAs (sgRNAs) per gene, scaled. The Y-axis shows the p-value as calculated by MAGeCK. Red are negative regulators (depleted in Foxp3 low cells), while blue dots show all positive regulators (enriched in Foxp3 low cells) defined by FDR <0.5 and Z-score >0.5.

FIG. 2c (top panel) shows the distribution of sgRNA-level log-fold changes (LFC) values of Foxp3 low over Foxp3 high cells for 2,000 guides. FIG. 2c (Bottom panel) shows the LFC for all four individual sgRNAs targeting genes enriched in Foxp3 low cells (blue lines) and depleted genes (red lines), overlaid on grey gradient depicting the overall distribution.

FIG. 2d shows a schematic of experimentally determined and predicted protein-protein interactions between top hits, 16 negative regulators (red) and 25 positive regulators (red), generated by STRING-db. Black lines connect proteins that interact and dotted lines depict known protein complexes.

FIG. 2e shows Foxp3 expression 5 days post electroporation of Cas9 RNPs in mouse Tregs as measured by flow cytometry of top screen hits.

FIG. 2f shows the mean fluorescence intensity (MFI) of Foxp3 from data in FIG. 2e.

FIG. 2g shows a representative histogram showing MFI of FOXP3 and CD25 from human Tregs.

FIG. 2h shows the statistical analysis of FOXP3 MFI from human Tregs in 6 biological replicates.

FIG. 2i is an S-curve for hits from the screen. The X-axis shows rank score for gene-level LFC; rank 1 is the top negative hit (Sp1), and rank 493 is the top positive hit (Foxp3). Y-axis shows the gene-level LFC as calculated by MAGeCK. Red dots show selected negative hits (depleted in Foxp3 low cells), while blue dots show selected positive hits (enriched in Foxp3 low cells) within the top 20 ranked hits.

FIG. 2j shows that in a targeted screen of over 2000 gRNAs, sgRNAs targeting Foxp3 and Usp22 were enriched in Foxp3 low cells. Non-targeting sgRNAs were evenly distributed across the cell populations (black).

FIGS. 3a-g shows the design and quality control for targeted pooled CRISPR screen in primary mouse Tregs. (a) Design strategy for selection of genes for unbiased targeted library. Genes were selected based on gene ontology (GO) annotation and then sub-selected based on highest expression across any CD4 T cell subset for a total of 2,000 sgRNAs; (b) MSCV expression vector with Thy1.1 reporter used for retroviral transduction of the sgRNA library; (c) Detailed timeline schematic of the 12-day targeted screen pipeline. Arrows indicate when the cells were split and media was replenished; (d) Retroviral transduction efficiency of the targeted library in primary mouse Tregs shown by Thy1.1 surface expression measured by flow cytometry. The infection was scaled to achieve a high efficiency multiplicity of infection; (e) Foxp3 expression from screen input, output and control cells measured by flow cytometry. Top: Foxp3 expression from input Foxp3+ purified Tregs as measured by GFP expression on Day 0. Middle: Foxp3 expression as measured by endogenous intracellular staining from control Tregs (not transduced with library) on Day 12. Bottom: Foxp3 expression as measured by endogenous intracellular staining from screen Tregs (transduced with library) on Day 12; (f) Targeted screen (2,000 guides) shows that sgRNAs targeting Foxp3 and Usp22 were enriched in Foxp3 low cells (blue). Non-targeting control (NT Ctrl) sgRNAs were evenly distributed across the cell populations (black). (g) Distribution of read counts after next generation sequencing of sgRNAs of sorted cell populations, Foxp3 high and Foxp3 low.

FIGS. 4a-g shows validation of gene targets that regulate Foxp3 expression in primary mouse and human Tregs using Cas9 RNP arrays. (a) Overview of orthogonal validation strategy using arrayed electroporation of Cas9 RNPs. (b) Representative flow plots depicting FOXP3 and CD25 expression 7 days post electroporation of Cas9 RNPs in human Tregs. The Foxp3hi CD25hi subpopulation is highlighted with a red gate. (c) Percentage of FOXP3⁺ cells from human Tregs in 6 biological replicates. (d) Percentage of FOXP3^{hi}CD25^{hi} cells from human Tregs in 6 biological replicates. (e) RNP controls in mouse Tregs collected 5 days post electroporation. Left: CD4 expression from CD4 RNP (cutting control) compared to NT control. Right: Foxp3 expression from CD4 knockout cells (left panel) compared to NT control. (f) Foxp3 expression 6 days after electroporation of Cas9 RNPs as measured by flow cytometry. Cells were pre-gated on lymphocytes, live cells, CD4+, CD25hi cells; (g) Statistical analysis of the mean fluorescence intensity (MFI) of Foxp3 from data in panel g. A two-way ANOVA with Holm-Sidak multiple comparisons test was used for statistical analysis. ** P≤0.01, **** P≤0.0001.

FIGS. 5a-b show validation of Rnf20 in primary mouse Tregs using Cas9 RNP array. (a) How cytometry histograms for 2 gRNAs targeting Rnf20 shows that Rnf20 knockout maintains stable Foxp3 expression. (b) Bar graph of Foxp3 MFI data from FIG. 5a.

FIG. 6 shows validation of USP22 regulation of Foxp3 expression in primary human Tregs using RNP arrays. (a) Foxp3 expression 7 days after electroporation of Cas9 RNPs as measured by flow cytometry. Cells were pre-gated on lymphocytes, live cells, CD4+, CD25hi cells. (b) Foxp3 MFI from data in panel a.

FIG. 7 shows that Usp22 and Atxn713 knockouts in mouse Tregs reduces Foxp3 expression, while Rnf20 knockdown maintains stable Foxp3 expression.

### DEFINITIONS

As used in this specification and the appended claims, the singular forms “a,” “an,” and “the” include plural reference unless the context clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” can refer to the segment of DNA involved in producing or encoding a polypeptide chain. It may include regions preceding and following the coding region (leader and trailer) as well as intervening sequences (introns) between individual coding segments (exons).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “inhibiting expression” refers to inhibiting or reducing the expression of a gene product, e.g., RNA or protein. As used throughout, the term “nuclear factor” refers to a protein that directly or indirectly alters expression of Foxp3, for example, a transcription factor. To inhibit or reduce the expression of a gene, the sequence and/or structure of the gene may be modified such that the gene would not be transcribed (for DNA) or translated (for RNA), or would not be transcribed or translated to produce a functional protein, for example, a polypeptide or protein encoded by a gene set forth in Table 1 or Table 2. Various methods for inhibiting or reducing expression are described in detail further herein. Some methods may introduce nucleic acid substitutions, additions, and/or deletions into the wild-type gene. Some methods may also introduce single or double strand breaks into the gene. To inhibit or reduce the expression of a protein, one may inhibit or reduce the expression of the gene or polynucleotide encoding the protein. In other embodiments, one may target the protein directly to inhibit or reduce the protein's expression using, e.g., an antibody or a protease. “Inhibited” expression refers to a decrease by at least 10% as compared to a reference control level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample).

The term “overexpressing” or “overexpression” refers to increasing the expression of a gene or protein. “Overexpression” refers to an increase in expression, for example, in increase in the amount of mRNA or protein expressed in a Treg cell, of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. Various methods for overexpression are known to those of skill in the art, and include, but are not limited to, stably or transiently introducing a heterologous polynucleotide encoding a protein (i.e., a nuclear factor set forth in Table 1 or Table 2) to be overexpressed into the cell or inducing overexpression of an endogenous gene encoding the protein in the cell.

As used herein the phrase “heterologous” refers to what is not found in nature. The term “heterologous sequence” refers to a sequence not normally found in a given cell in nature. As such, a heterologous nucleotide or protein sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

As used throughout, by subject is meant an individual. For example, the subject is a mammal, such as a primate, and, more specifically, a human Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

As used throughout, the term “targeted nuclease” refers to nuclease that is targeted to a specific DNA sequence in the genome of a cell to produce a strand break at that specific DNA sequence. The strand break can be single-stranded or double-stranded. Targeted nucleases include, but are not limited to, a Cas nuclease, a TAL-effector nuclease and a zinc finger nuclease.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. Table 3 shows exemplary gRNA sequences used in methods of the disclosure.

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p′759-′7′71, 22 Oct. 2015) and homologs thereof. Similarly, as used herein, the term “Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be subsitututed with a Cpf1 nuclease or any other guided nuclease.

As used herein, the phrase “modifying” refers to inducing a structural change in the sequence of the genome at a target genomic region in a Treg cell. For example, the modifying can take the form of inserting a nucleotide sequence into the genome of the cell. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region. “Modifying” can also refer to altering the expression of a nuclear factor in a Treg cell, for example inhibiting expression of a nuclear factor or overexpressing a nuclear factor in a Treg cell.

As used herein, the phrase “introducing” in the context of introducing a nucleic acid or a complex comprising a nucleic acid, for example, an RNP complex, refers to the translocation of the nucleic acid sequence or the RNP complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Introduction

Treg cells are a specialized subset of CD4+ T cells that suppress inflammation to maintain homeostasis and prevent autoimmunity. Treg cell development and function depend on expression of the master transcription factor Foxp3. While Treg cells have been thought to be irreversibly committed to suppressive functions, lineage tracing studies have revealed that Treg cells can exhibit plasticity. Treg cells that lose Foxp3 expression, termed ‘exTregs’, have been shown to acquire cytokine production capabilities of pro-inflammator effector T cells and exacerbate autoimmunity. However, the gene regulatory programs that promote or disrupt Foxp3 stability in Treg cells under various physiological conditions are not well understood. The inventors have identified nuclear factors that regulate expression of Foxp3, thereby altering Treg cell stability.

### II. Methods and Compositions

As described herein, the disclosure provides compositions and methods directed to modifying regulatory T (Treg) cell stability by inhibiting the expression of one or more nuclear factors and/or overexpressing one or more nuclear factors in a Treg cell. The disclosure also features compositions comprising the Treg cells having modified stability. A population of modified Treg cells that are destabilized may provide therapeutic benefits in treating cancer. A population of modified Treg cells that are stabilized may provide therapeutic benefits in treating autoimmune diseases.

The present disclosure is directed to compositions and methods for modifying the stability of regulatory T cells (also referred to as “Treg cells”). The inventors have discovered that by inhibiting the expression of one or more nuclear factors and/or overexpressing one or more nuclear factors, the stability of Treg cells may be altered. In some embodiments, the Treg cells may be destabilized by inhibiting the expression of one or more nuclear factors and/or overexpressing one or more nuclear factors, such that they may have less immunosuppressive effects and improved therapeutic benefits towards treating cancer. A population of destabilized Treg cells may be used to enhance or improve various cancer therapies or Treg cells of an individual having cancer can be targeted to destabilize the Treg cells. In other embodiments, Treg cells may be stabilized by inhibiting the expression of one or more nuclear factors and/or overexpressing one or more nuclear factors, such that they may have more immunosuppressive effects and improved therapeutic benefits towards treating an autoimmune disease. A population of stabilized Treg cells may be used to treat or alleviate autoimmune diseases or Treg cells of an individual having an autoimmune disease can be targeted to stabilize the Treg cells.

Examples of nuclear factors whose expression may be altered to modify the stability of Treg cells in the methods described herein include, but are not limited to the nuclear factors set forth in Table 1 and Table 2. In some embodiments, the present invention provides a method of increasing regulatory T (Treg) cell stability, the method comprising: inhibiting expression of one or more nuclear factors set forth in Table 1 and/or overexpressing one or more nuclear factors set forth in Table 2 in the Treg cell. Inhibition of one or more nuclear factors set forth in Table 1 and/or overexpression of one or more nuclear factors set forth in Table 2 may increase Foxp3 expression in the Treg cell or stabilize Foxp3 expression (e.g., in an inflammatory environment that would otherwise result in Foxp3 expression reduction), thereby increasing stability of the Treg cell.

In other embodiments, the present invention provides a method of decreasing Treg cell stability, the method comprising: inhibiting expression of one or more nuclear factors set forth in Table 2 and/or overexpressing one or more nuclear factors set forth in Table 1, in the Treg cell. Inhibition of one or more nuclear factors set forth in Table 2 and/or overexpression of one or more nuclear factors set forth in Table 1 may decrease Foxp3 expression in the Treg cell, thereby decreasing stability of the Treg cell. Table 1 provides nuclear factors that, when inhibited, increase Foxp3 expression. Overexpression of a nuclear factor set forth in Table 1 may decrease Foxp3 expression. In some embodiments, expression of an amino acid sequence having at least about 80%, 85%, 90%, 95% or 99% identity to an amino acid sequence set forth in Table 1 is inhibited. In some embodiments, an amino acid sequence having at least about 80%, 85%, 90%, 95% or 99% identity to an amino acid sequence set forth in Table 1 is overexpressed. Table 2 provides nuclear factors that, when inhibited, decrease Foxp3 expression. Overexpression of a nuclear factor set forth in Table 2 may increase Foxp3 expression. In some embodiments, expression of an amino acid sequence having at least about 80%, 85%, 90%, 95% or 99% identity to an amino acid sequence set forth in Table 2 is inhibited. In some embodiments, an amino acid sequence having at least about 80%, 85%, 90%, 95% or 99% identity to an amino acid sequence set forth in Table 2 is overexpressed. It is understood that, when referring to one or more nuclear factors set forth in Table 1 or Table 2, this can be the protein, i.e., the nuclear factor, or the polynucleotide encoding the nuclear factor.

TABLE 1
Nuclear factors that can be inhibited to increase Foxp3 expression or
overexpressed to decrease Foxp3 expression.

| | GenBank | | | |
|---|---|---|---|---|
| Gene/protein | Accession No. | Definition | Length | Amino acid sequence |
| Sp1 | NP_001238754.1 | transcription factor Sp1 | 737 aa | msdqdhsmde mtavvkiekg vggnnggngn gggafsqars sstgsssstg gggqgangwq |
| | | isoform c [Homo sapiens]. | | iissssgatp tskeqsgsst ngsngsessk nrtvsggqyv vaaapnlqnq qvltglpgvm |
| | | | | pniqyqvipq fqtvdgqqlq faatgaqvqq dgsgqiqiip ganqqiitnr gsggniiaam |
| | | | | pnllqqavpl qglannvlsg qtqyvtnvpv alngnitllp vnsysaatlt pssqavtiss |
| | | | | sgsqesgsqp vtsgttissa slvssqasss sfftnansys tttttsnmgi mnfttsgssg |
| | | | | tnsqgqtpqr vsglqgsdal niqqnqtsgg slqagqqkeg eqnqqtqqqq iliqpqlvqg |
| | | | | gqalqalqaa plsgqtfttq aisqetlqnl qlqavpnsgp iiirtptvgp ngqvswqtlq |
| | | | | lqnlqvqnpq aqtitlapmq gvslgqtsss nttltpiasa asipagtvtv naaqlssmpg |
| | | | | lqtinlsalg tsgiqvhpiq glplaianap gdhgaqlglh gaggdgihdd taggeegens |
| | | | | pdaqpqagrr trreactcpy ckdsegrgsg dpgkkkqhic hiqgcgkvyg ktshlrahlr |
| | | | | whtgerpfmc twsycgkrft rsdelqrhkr thtgekkfac pecpkrfmrs dhlskhikth |
| | | | | qnkkggpgva lsvgtlplds gagsegsgta tpsalittnm vameaicpeg iarlansgin |
| | | | | vmqvadlqsi nisgngf (SEQ ID NO: 1) |
| Rnf20 | NP_062538.5 | E3 ubiquitin-protein ligase | 975 aa | msgignkraa gepgtsmppe kkaavedsgt tvetiklggv ssteeldirt lqtknrklae |
| | | BRE1A [Homo sapiens]. | | mldqrqaied elrehiekle rrqatddasl livnrywsqf deniriilkr ydleqglgdl |
| | | | | lterkalvvp epepdsdsnq erkddrerge gqepafsfla tlasssseem esqlqerves |
| | | | | srraysqivt vydklqekve llsrklnsgd nliveeavqe lnsflaqenm rlqeltdllq |
| | | | | ekhrtmsqef sklqskveta esrvsvlesm iddlqwdidk irkreqrinr hlaevlervn |
| | | | | skgykvygag sslyggtiti narkfeemna eleenkelaq nrlceleklr qdfeevttqn |
| | | | | eklkvelrsa veqvvketpe yrcmqsqfsv lyneslqlka hldeartllh gtrgthqhqv |
| | | | | elierdevsl hkklrteviq ledtlaqvrk eyemlriefe qtlaaneqag pinremrhli |
| | | | | sslqnhnhql kgevlrykrk lreaqsdlnk trlrsgsall qsqsstedpk depaelkpds |
| | | | | edlssqssas kasqedanei kskrdeeere rerrekerer ererekeker erekqklkes |
| | | | | ekerdsakdk ekgkhddgrk keaeiikqlk ielkkaqesq kemkllldmy rsapkeqrdk |
| | | | | vqlmaaekks kaeledlrqr lkdledkekk enkkmadeda lrkiraveeq ieylqkklam |
| | | | | akqeeealls emdvtgqafe dmqeqnirlm qqlrekddan fklmseriks nqihkllkee |
| | | | | keeladqvlt lktqvdaqlq vvrkleekeh llqsnigtge kelglrtqal emnkrkamea |
| | | | | aqladdlkaq lelaqkklhd fqdeivensv tkekdmfnfk raqedisrlr rklettkkpd |
| | | | | nvpkcdeilm eeikdykarl tcpccnmrkk davltkcfhv fcfecvktry dtrqrkcpkc |
| | | | | naafgandfh riyig (SEQ ID NO: 2) |
| Rfx7 | NP_073752.5 | DNA-binding protein RFX7 | 1460 aa | maeeqqqppp qqpdahqqlp psapnsgval palvpglpgt easalqhkik nsicktvqsk |
| | | [Homo sapiens]. | | vdcilqevek ftdleklyly lqlpsglsng eksdqnamss sraqqmhafs wirntleehp |
| | | | | etslpkqevy deyksycdnl gyhplsaadf gkimknvfpn mkarrlgtrg kskycysglr |
| | | | | kkafvhmptl pnldfhktgd glegaepsgq lqnideevis sacrlvcewa qkvlsqpfdt |
| | | | | vlelarflvk shyigtksma altvmaaapa gmkgitqpsa fiptaesnsf qpqvktlpsp |
| | | | | idakqqlqrk iqkkqqeqkl qsplpgesaa kksesatsng vtnlpngnps ilspqpigiv |
| | | | | vaavpspipv qrtrqlvtsp spmsssdgkv lpinvqvvtq hmqsvkqapk tpqnvpaspg |
| | | | | gdrsarhryp qilpkpants altirspttv lftsspikta vvpashmssl nvvkmttisl |
| | | | | tpsnsntplk hsasyssatg tteesrsvpq ikngsvvslq spgsrsssag gtsavevkve |
| | | | | petssdehpv qcqensdeak apqtpsallg qksntdgalq kpsnegviei katkvcdqrt |
| | | | | kcksrcneml pgtstgnnqs titlsvasqn ltftsssspp ngdsinkdpk lctksprkrl |
| | | | | sstlqetqvp pvkkpiveql saatiegqkq gsvkkdqkvp hsgktegsta gaqipskvsv |
| | | | | nvsshiganq pinssalvis dsaleqqttp ssspdikvkl egsvflldsd sksvgsfnpn |
| | | | | gwqqitkdse fisasceqqq disvmtipeh sdindleksv welegmpqdt ysqqlhsqiq |
| | | | | esslnqiqah ssdqlplqse lkefepsysq tnesyfpfdd eltqdsivee lvlmeqqmsm |
| | | | | nnshsygncl gmtlqsqsvt pgapmsshts sthfyhpihs ngtpihtptp tptptptptp |
| | | | | tptptsemia gsqslsresp csrlaqttpv dsalgssrht pigtphsncs ssvppspvec |
| | | | | rnpfaftpis ssmayhdasi vssspvkpmq rpmathpdkt klewmnngys gvgnssysgh |
| | | | | gilpsyqelv edrfrkphaf avpgqsyqsq srhhdtnfgr ltpvspvqhq gatvnntnkq |
| | | | | egfavpapld nkgtnssass nfrcrsyspa vhrqrnlsgs tlypvsnipr snvtpfgspv |
| | | | | tpevhvftnv htdacannia qrsqsvpltv mmqtafpnal qkqanskkit nvllskldsd |
| | | | | nddavrglgm nnlpsnytar mnitqileps tvfpsanpqn midsstsvye fqtpsyltks |
| | | | | nstgqinfsp gdnqaqseig eqqldfnstv kdllsgdslq tnqqlvgqga sdltntasdf |
| | | | | ssdirlssel sgsindlntl dpnllfdpgr qqgqddeatl eelkndplfq qicsesmnsm |
| | | | | tssgfewies kdhptvemlg (SEQ ID NO: 3) |
| Srf | NP_003122.1 | serum response factor | 508 aa | mlptqagaaa algrgsalgg slnrtptgrp gggggtrgan ggrvpgngag lgpgrlerea |
| | | isoform 1 [Homo sapiens]. | | aaaaattpap tagalysgse gdsesgeeee lgaerrglkr slsemeigmv vggpeasaaa |
| | | | | tggygpvsga vsgakpgkkt rgrvkikmef idnklrrytt fskrktgimk kayelstltg |
| | | | | tqvlllvase tghvytfatr klqpmitset gkaliqtcln spdspprsdp ttdqrmsatg |
| | | | | feetdltyqv sesdssgetk dtlkpaftvt nlpgttstiq tapststtmq vssgpsfpit |
| | | | | nylapvsasv spsayssang tvlkstgsgp vssgglmqlp tsffimpgga vaqqvpvqai |
| | | | | qvhqapqqas psrdsstdlt qtsssgtvtl patimtssvp ttvgghmmyp sphavmyapt |
| | | | | sglgdgsltv lnafsqapst mqvshsqvqe pggvpqvflt assgtvqipv savqlhqmav |
| | | | | igqqagsssn ltelqvvnld tahstkse (SEQ ID NO: 4) |
| Elp2 | NP_001229804.1 | elongator complex protein 2 | 891 aa | mvapvletsh vfccpnrvrg vinwssgprg llafgtscsv vlydplkrvv vtnlnghtar |
| | | isoform 1 [Homo sapiens]. | | vnciqwickq dgspstelvs ggsdnqvihw eiednqllka vhlqghegpv yavhavyqrr |
| | | | | tsdpalctli vsaaadsavr lwskkgpevm clqtlnfgng falalclsfl pntdvtwktg |
| | | | | qvergrawkp paslalcsrs cdsmvscyas ilckalwkek lhtfwhhnri sflpsafrpi |
| | | | | pilacgnddc rihifaqqnd qfqkvlslcg hedwirgvew aafgrdlfla scsqdcliri |
| | | | | wklyikstsl etqdddnirl kentftiene svkiafavtl etvlaghenw vnavhwqpvf |
| | | | | ykdgvlqqpv rllsasmdkt milwapdees gvwleqvrvg evggntlgfy dcqfnedgsm |
| | | | | iiahafhgal hlwkqntvnp rewtpeivis ghfdgvqdlv wdpegefiit vgtdqttrlf |
| | | | | apwkrkdqsq vtwheiarpq ihgydlkcla minrfqfvsg adekvlrvfs aprnfvenfc |
| | | | | aitgqslnhv lcnqdsdlpe gatvpalgls nkavfqgdia sqpsdeeell tstgfeyqqv |
| | | | | afqpsiltep ptedhllqnt lwpevqklyg hgyeifcvtc nssktllasa ckaakkehaa |
| | | | | iilwnttswk qvqnlvfhsl tvtqmafspn ekfllaysrd rtwslwkkqd tispefepvf |
| | | | | slfaftnkit svhsriiwsc dwspdskyff tgsrdkkvvv wgecdstddc iehnigpcss |
| | | | | vldvggavta vsvcpvlhps qryvvavgle cgkiclytwk ktdqvpeind wthcvetsqs |
| | | | | qshtlairkl cwkncsgkte qkeaegaewl hfascgedht vkihrvnkca l (SEQ ID NO: 5) |
| Nsd1 | NP_758859.1 | histone-lysine N- | 2427 aa | mplktrtals ddpdsststl gnmlelpgts ssstsqelpf cqpkkkstpl kyevgdliwa |
| | | methyltransferase, H3 | | kfkrrpwwpc ricsdplint hskmkvsnrr pyrqyyveaf gdpserawva gkaivmfegr |
| | | lysine-36 and H4 lysine-20 | | hqfeelpvlr rrgkqkekgy rhkvpqkils kweasvglae qydvpkgskn rkcipgsikl |
| | | specific isoform a [Homo | | dseedmpfed ctndpesehd lllngclksl afdsehsade kekpcaksra rkssdnpkrt |
| | | sapiens]. | | svkkghiqfe ahkderrgki penlglnfis gdisdtqasn elsrianslt gsntapgsfl |
| | | | | fsscgkntak kefetsngds llglpegali skcsreknkp qrslvcgskv klcyigagde |
| | | | | ekrsdsisic ttsddgssdl dpiehssesd nsvleipdaf drtenmlsmq knekikysrf |
| | | | | aatntrvkak qkplisnsht dhlmgctksa epgtetsqvn lsdlkastiv hkpqsdftnd |
| | | | | alspkfnlss sissenslik ggaanqallh skskqpkfrs ikckhkenpv maeppvinee |
| | | | | cslkccssdt kgsplasisk sgkvdglkll nnmhektrds sdietavvkh vlselkelsy |
| | | | | rslgedvsds gtskpskpll fssassqnhi piepdykfst llmmlkdmhd sktkeqrlmt |
| | | | | aqnlvsyrsp grgdcstnsp vgvskvlvsg gsthnsekkg dgtqnsanps psggdsalsg |
| | | | | elsaslpgll sdkrdlpasg ksrsdcvtrr ncgrskpssk lrdafsaqmv kntvnrkalk |
| | | | | terkrklnql psvtldavlq gdrerggslr ggaedpsked plqimghlts edgdhfsdvh |
| | | | | fdskvkqsdp gkisekglsf engkgpelds vmnsendeln gvnqvvpkkr wqrinqrrtk |
| | | | | prkrmnrfke kensecafry llpsdpvqeg rdefpehrtp sasileeplt eqnhadclds |
| | | | | agprinvcdk ssasigdmek epgipsltpq aelpepavrs ekkrlrkpsk wlleyteeyd |
| | | | | qifapkkkqk kvqeqvhkvs srceeeslla rgrssaqnkq vdenslistk eeppvlerea |
| | | | | pflegplaqs elggghaelp qltlsvpvap evsprpales eellvktpgn yeskrqrkpt |
| | | | | kkllesndld pgfmpkkgdl glskkcyeag hlengitesc atsyskdfgg gttkifdkpr |
| | | | | krkrqrhaaa kmqckkvknd dsskeipgse gelmphrtat spketveegv ehdpgmpask |
| | | | | kmqgerggga alkenvcqnc eklgelllce aqccgafhle clgltemprg kficnecrtg |
| | | | | ihtcfvckqs gedvkrcllp lcgkfyheec vqkypptvmq nkgfrcslhi citchaanpa |
| | | | | nvsaskgrlm rcvrcpvayh andfclaags kilasnsiic pnhftprrgc rnhehvnvsw |
| | | | | cfvcseggsl lccdscpaaf hreclnidip egnwycndck agkkphyrei vwvkvgryrw |
| | | | | wpaeichpra vpsnidkmrh dvgefpvlff gsndylwthq arvfpymegd vsskdkmgkg |
| | | | | vdgtykkalq eaaarfeelk aqkelrqlqe drkndkkppp ykhikvnrpi grvqiftadl |
| | | | | seiprcncka tdenpcgids ecinrmllye chptvcpagg rcqnqcfskr qypeveifrt |
| | | | | lqrgwglrtk tdikkgefvn eyvgelidee ecrariryaq ehditnfyml tldkdriida |
| | | | | gpkgnyarfm nhccqpncet qkwsvngdtr vglfalsdik agteltfnyn leclgngktv |
| | | | | ckcgapncsg flgvrpknqp iateekskkf kkkqqgkrrt qgeitkered ecfscgdagq |
| | | | | lvsckkpgcp kvyhadclnl tkrpagkwec pwhqcdicgk eaasfcemcp ssfckqhreg |
| | | | | mlfiskldgr lsctehdpcg pnplepgeir eyvpppvplp pgpsthlaeq stgmaaqapk |
| | | | | msdkppadtn qmlslskkal agtcqrpllp erplertdsr pqpldkvrdl agsgtksqsl |
| | | | | vssqrpldrp pavagprpql sdkpspvtsp ssspsvrsqp lerplgtadp rldksigaas |
| | | | | prpqslekts vptglrlppp drllitsspk pqtsdrptdk phaslsqrlp ppekvlsavv |
| | | | | qtivakekal rpvdqntqsk nraalvmdli dltprqkera asphqvtpqa dekmpvless |
| | | | | swpaskglgh mpravekgcv sdplqtsgka aapsedpwqa vksltqarll sqppakafly |
| | | | | epttqasgra sagaeqtpgp lsqspglvkq akqmvggqql palaaksgqs frslgkapas |
| | | | | lpteekklvt teqspwalgk assraglwpi vagqtlaqsc wsagstqtla qtcwslgrgq |
| | | | | dpkpeqntlp alnqapsshk caeseqk (SEQ ID NO: 6) |
| Smarcb1 | NP_001349806.1 | SWI/SNF-related matrix- | 403 aa | mmmmalsktf gqkpvkfqle ddgefymigs evgnylrmfr gslykrypsl wrrlatveer |
| | | associated actin-dependent | | kkivasshgk ktkpntkdhg yttlatsvtl lkaseveeil dgndekykav sistepptyl |
| | | regulator of chromatin | | reqkakrnsq wvptlpnssh hldavpcstt inrnrmgrdk krtfplwcgc iaaltlrads |
| | | subfamily B member 1 | | alvlhfddhd pavihenasq pevlvpirld meidgqklrd aftwnmnekl mtpemfseil |
| | | isoform d [Homo sapiens]. | | cddldlnplt fvpaiasair qqiesyptds iledqsdqry iiklnihvgn islvdqfewd |
| | | | | msekenspek falklcselg lggefvttia ysirgqlswh qktyafsenp lptveiairn |
| | | | | tgdadqwcpl letltdaeme kkirdqdrnt rrmrrlanta paw (SEQ ID NO: 7) |
| Klf2 | NP_057354.1 | Krueppel-like factor 2 | 355 aa | malsepilps fstfaspere rglqerwpra epesggtddd lnsvldfils mgldglgaea |
| | | [Homo sapiens]. | | apeppppppp pafyypepga pppysapagg lvsellrpel daplgpalhg rfllappgrl |
| | | | | vkaeppeadg gggygcapgl trgprglkre gapgpaascm rgpggrpppp pdtpplspdg |
| | | | | parlpapgpr asfpppfggp gfgapgpglh yappappafg lfddaaaaaa alglappaar |
| | | | | glltppaspl elleakpkrg rrswprkrta thtcsyagcg ktytksshlk ahlrthtgek |
| | | | | pyhcnwdgcg wkfarsdelt rhyrkhtghr pfqchlcdra fsrsdhlalh mkrhm (SEQ ID NO: 8) |
| Ctcf | NP_001350845.1 | transcriptional repressor | 725 aa | megdaveaiv eesetfikgk erktyqrrre ggqeedachl pqnqtdggev vqdvnssvqm |
| | | CTCF isoform 3 [Homo | | vmmeqldptl lqmktevmeg tvapeaeaav ddtqiitlqv vnmeeqpini gelqlvqvpv |
| | | sapiens]. | | pvtvpvatts veelqgayen evskeglaes epmichtlpl pegfqvvkvg angevetleq |
| | | | | gelppqedps wqkdpdyqpp aldakktkks klryteegkd vdvsvydfee eqqegllsev |
| | | | | naekvvgnmk ppkptkikkk gvkktfqcel csytcprrsn ldrhmkshtd erphkchlcg |
| | | | | rafrtvtllr nhlnthtgtr phkcpdcdma fvtsgelvrh rrykhthekp fkcsmcdyas |
| | | | | vevsklkrhi rshtgerpfq cslcsyasrd tyklkrhmrt hsgekpyecy icharftqsg |
| | | | | tmkmhilqkh tenvakfhcp hcdtviarks dlgvhlrkqh syieqgkkcr ycdavfhery |
| | | | | aliqhqkshk nekrfkcdqc dyacrqerhm imhkrthtge kpyacshcdk tfrqkqlldm |
| | | | | hfkryhdpnf vpaafvcskc gktftrrntm arhadncagp dgvegengge tkkskrgrkr |
| | | | | kmrskkedss dsenaepdld dnedeeepav eiepepepqp vtpapppakk rrgrppgrtn |
| | | | | qpkqnqpiiq vedqntgaie niivevkkep daepaegeee eaqpaatdap ngdltpemil |
| | | | | smmdr (SEQ ID NO: 9) |
| Satb1 | NP_001309804.1 | DNA-binding protein | 763 aa | mdhlneatqg kehsemsnnv sdpkgppaki arleqngspl grgrlgstga kmqgvplkhs |
| | | SATB1 isoform 1 [Homo | | ghlmktnlrk gtmlpvfcvv ehyenaieyd ckeehaefvl vrkdmlfnql iemallslgy |
| | | sapiens]. | | shssaaqakg liqvgkwnpv plsyvtdapd atvadmlqdv yhvvtlkiql hscpkledlp |
| | | | | peqwshttvr nalkdllkdm nqsslakecp lsqsmissiv nstyyanvsa akcqefgrwy |
| | | | | khfkktkdmm vemdslsels qqganhvnfg qqpvpgntae qppspaqlsh gsqpsvrtpl |
| | | | | pnlhpglvst pispqlvnqq lvmaqllnqq yavnrllaqq slnqqylnhp ppvsrsmnkp |
| | | | | leqqvstnte vsseiyqwvr delkragisq avfarvafnr tqgllseilr keedpktasq |
| | | | | sllvnlramq nflqlpeaer driyqderer slnaasamgp aplistppsr ppqvktatia |
| | | | | terngkpenn tmninasiyd eiqqemkrak vsqalfakva atksqgwlce llrwkedpsp |
| | | | | enrtlwenls mirrflslpq perdaiyeqe snavhhhgdr pphiihvpae qiqqqqqqqq |
| | | | | qqqqqqqapp ppqpqqqpqt gprlpprqpt vaspaesdee nrqktrprtk isvealgilq |
| | | | | sfiqdvglyp deeaiqtlsa qldlpkytii kffqnqryyl khhgklkdns glevdvaeyk |
| | | | | eeellkdlee svqdkntntl fsvkleeels vegntdintd lkd (SEQ ID NO: 10) |

TABLE 2
Nuclear factors that can be inhibited to decrease Foxp3 expression or overexpressed
to increase Foxp3 expression.

| | GenBank | | | | |
|---|---|---|---|---|---|
| Gene/protein | Accession No. | Definition | Length | Amino acid sequence | |
| Foxp3 | NP_001107849.1 | forkhead box protein | 396 aa | 1 | mpnprpgkps apslalgpsp gaspswraap kasdllgarg pggtfqgrdl rggahassss |
| | | P3 isoform b [Homo | | 61 | lnpmppsqlq lstvdahart pvlqvhples pamisltppt tatgvfslka rpglppginv |
| | | sapiens]. | | 121 | aslewvsrep allctfpnps aprkdstlsa vpqssyplla ngvckwpgce kvfeepedfl |
| | | | | 181 | khcqadhlld ekgraqcllq remvqsleqq lvlekeklsa mqahlagkma ltkassvass |
| | | | | 241 | dkgsccivaa gsqgpvvpaw sgpreapdsl favrrhlwgs hgnstfpefl hnmdyfkfhn |
| | | | | 301 | mrppftyatl irwaileape kqrtlneiyh wftrmfaffr nhpatwknai rhnlslhkcf |
| | | | | 361 | vrvesekgav wtvdelefrk krsqrpsrcs nptpgp (SEQ ID NO: 11) |
| Usp22 | NP_056091 | ubiquitin carboxyl- | 525 aa | 1 | mvsrpepege amdaelavap pgcshlgsfk vdnwkqnlra iyqcfvwsgt aearkrkaks |
| | | terminal hydrolase 22 | | 61 | cichvegvhl nrlhsclycv ffgeftkkhi hehakakrhn laidlmyggi ycflcqdyiy |
| | | [Homo sapiens]. | | 121 | dkdmeiiake eqrkawkmqg vgekfstwep tkrelellkh npkrrkitsn ctiglrglin |
| | | | | 181 | lgntcfmnci vqalthtpll rdfflsdrhr cemqspsscl vcemsslfqe fysghrsphi |
| | | | | 241 | pykllhlvwt harhlagyeq qdahefliaa ldvlhrhckg ddngkkannp nhcnciidqi |
| | | | | 301 | ftgglqsdvt cqvchgvstt idpfwdisld lpgsstpfwp lspgsegnvv ngeshvsgtt |
| | | | | 361 | tltdclrrft rpehlgssak ikcsgchsyq estkqltmkk lpivacfhlk rfehsaklrr |
| | | | | 421 | kittyvsfpl eldmtpfmas skesrmngqy qqptdslnnd nkyslfavvn hqgtlesghy |
| | | | | 481 | tsfirqhkdq wfkcddaiit kasikdvlds egyllfyhkq fleye (SEQ ID NO: 12) |
| Cbfb | NP_074036.1 | core-binding factor | 187 aa | 1 | mprvvpdqrs kfeneeffrk lsreceikyt gfrdrpheer qarfqnacrd grseiafvat |
| | | subunit beta isoform 1 | | 61 | gtnlslqffp aswqgeqrqt psreyvdler eagkvylkap milngvcviw kgwidlqrld |
| | | [Homo sapiens]. | | 121 | gmgclefdee raqqedalaq qafeearrrt refedrdrsh reemearrqq dpspgsnlgg |
| | | | | 181 | gddlklr (SEQ ID NO: 13) |
| Runxl | NP_001001890.1 | runt-related | 453 aa | 1 | mripvdasts rrftppstal spgkmsealp lgapdagaal agklrsgdrs mvevladhpg |
| | | transcription factor 1 | | 61 | elvrtdspnf lcsvlpthwr cnktlpiafk vvalgdvpdg tivtvmagnd enysaelrna |
| | | isoform AML1b | | 121 | taamknqvar fndlrfvgrs grgksftlti tvftnppqva tyhraikitv dgpreprrhr |
| | | [Homo sapiens]. | | 181 | qklddqtkpg slsfserlse leqlrrtamr vsphhpaptp npraslnhst afnpqpqsqm |
| | | | | 241 | qdtrqiqpsp pwsydqsyqy lgsiaspsvh patpispgra sgmttlsael ssrlstapdl |
| | | | | 301 | tafsdprqfp alpsisdprm hypgaftysp tpvtsgigig msamgsatry htylpppypg |
| | | | | 361 | ssqaqggpfq asspsyhlyy gasagsyqfs mvggersppr ilppctnast gsallnpslp |
| | | | | 421 | nqsdvveaeg shsnsptnma psarleeavw rpy (SEQ ID NO: 14) |
| Myc | NP_001341799.1 | myc proto-oncogene | 453 aa | 1 | mdffrvvenq ppatmpinvs ftnrnydldy dsvqpyfycd eeenfyqqqq qselqppaps |
| | | protein isoform 2 | | 61 | ediwkkfell ptpplspsrr sglcspsyva vtpfslrgdn dggggsfsta dqlemvtell |
| | | [Homo sapiens]. | | 121 | ggdmvnqsfi cdpddetfik niiiqdcmws gfsaaaklvs eklasyqaar kdsgspnpar |
| | | | | 181 | ghsvcstssl ylqdlsaaas ecidpsvvfp ypindssspk scasqdssaf spssdsllss |
| | | | | 241 | tesspqgspe plvlheetpp ttssdseeeq edeeeidvvs vekrqapgkr sesgspsagg |
| | | | | 301 | hskpphsplv lkrchvsthq hnyaappstr kdypaakrvk ldsvrvlrqi snnrkctspr |
| | | | | 361 | ssdteenvkr rthnvlerqr rnelkrsffa lrdqipelen nekapkvvil kkatayilsv |
| | | | | 421 | qaeeqklise edllrkrreq lkhkleqlrn sea (SEQ ID NO: 15) |
| Ss18 | NP_001295130.1 | protein SSXT isoform | 395 aa | 1 | mlddnnhliq cimdsqnkgk tsecsqyqqm lhtnlvylat iadsnqnmqs llpapptqnm |
| | | 3 [Homo sapiens]. | | 61 | pmgpggmnqs gppppprshn mpsdgmvggg ppaphmqnqm ngqmpgpnhm pmqgpgpnql |
| | | | | 121 | nmtnssmnmp ssshgsmggy nhsvpssqsm pvqnqmtmsq gqpmgnygpr pnmsmqpnqg |
| | | | | 181 | pmmhqqppsq qynmpqgggq hyqgqqppmg mmgqvnqgnh mmgqrqippy rppqqgppqq |
| | | | | 241 | ysgqedyygd qyshggqgpp egmnqqyypd ghndygyqqp sypeqgydrp yedssqhyye |
| | | | | 301 | ggnsqygqqq dayqgpppqq gyppqqqqyp gqqgypgqqq gygpsqggpg pqypnypqgq |
| | | | | 361 | gqqyggyrpt qpgppqppqq rpygydqgqy gnyqq (SEQ ID NO: 16) |
| Med30 | NP_001350111.1 | mediator of RNA | 157 aa | 1 | mstpplaasg mapgpfagpq aqqaarevnt aslcrigqet vqdivyrtme ifqllrnmql |
| | | polymerase II | | 61 | pngvtyhtgt yqdrltklqd nlrqlsvlfr klrlvydkcn encggmdpip veqlipyvee |
| | | transcription subunit 30 | | 121 | dgsknddrag pprfaseerr eiaevnkals svpeflp (SEQ ID NO: 17) |
| | | isoform 3 | | | |
| Atxn713 | NP_064603.1 | ataxin-7-like protein 3 | 354 aa | 1 | mkmeemslsg ldnskleaia qeiyadlved sclgfcfevh ravkcgyffl ddtdpdsmkd |
| | | isoform a [Homo | | 61 | feivdqpgld ifgqvfnqwk skecvcpncs rsiaasrfap hlekclgmgr nssrianrri |
| | | sapiens]. | | 121 | ansnnmnkse sdqednddin dndwsygsek kakkrksdkl wylpfqnpns prrskslkhk |
| | | | | 181 | ngelsnsdpf kynnstgisy etlgpeelrs llttqcgvis ehtkkmctrs lrcpqhtdeq |
| | | | | 241 | rrtvriyflg psavlpeves sldndsfdmt dsqalisrlq wdgssdlsps dsgssktsen |
| | | | | 301 | qgwglgtnss esrktkkkks hlslvgtasg lgsnkkkkpk ppapptpsiy ddin (SEQ ID NO: 18) |
| Med12 | NP_005111.2 | mediator of RNA | 2177 aa | 1 | maafgilsye hrplkrprlg ppdvypqdpk qkedeltaln vkqgfnnqpa vsgdehgsak |
| | | polymerase II | | 61 | nvsfnpakis snfssiiaek lrcntlpdtg rrkpqvnqkd nfwlvtarsq saintwftdl |
| | | transcription subunit 12 | | 121 | agtkpltqla kkvpifskke evfgylakyt vpvmraawli kmtcayyaai setkvkkrhv |
| | | [Homo sapiens]. | | 181 | dpfmewtqii tkylweqlqk maeyyrpgpa gsggcgstig plphdvevai rqwdytekla |
| | | | | 241 | mfmfqdgmld rhefltwvle cfekirpged ellklllpll lrysgefvqs aylsrrlayf |
| | | | | 301 | ctrrlalqld gvsshsshvi saqststlpt tpapqpptss tpstpfsdll mcpqhrplvf |
| | | | | 361 | glscilqtil lccpsalvwh ysltdsrikt gspldhlpia psnlpmpegn saftqqvrak |
| | | | | 421 | lreieqqike rgqavevrws fdkcqeatag ftigrvlhtl evldshsfer sdfsnsldsl |
| | | | | 481 | cnrifglgps kdgheissdd davvsllcew aysclusgrh ramvvaklle krqaeieaer |
| | | | | 541 | cgeseaadek gsiasgslsa psapifqdvl lqfldtqapm ltdprseser veffnlvllf |
| | | | | 601 | celirhdvfs hnmytctlis rgdlafgapg prppspfddp addpehkeae gsssskledp |
| | | | | 661 | glsesmdidp sssvlfedme kpdfslfspt mpcegkgsps pekpdvekev kpppkekieg |
| | | | | 721 | tlgvlydqpr hvqyathfpi pqeescshec nqrlvvlfgv gkqrddarha ikkitkdilk |
| | | | | 781 | vinrkgtaet dqlapivpin pgdltflgge dgqkrrrnrp eafptaedif akfqhlshyd |
| | | | | 841 | qhqvtaqvsr nvleqitsfa lgmsyhlplv qhvqfifdlm eyslsisgli dfaiqllnel |
| | | | | 901 | svveaelllk ssdlvgsytt slcicivavl rhyhacliln qdqmaqvfeg legyvkhgmn |
| | | | | 961 | rsdgssaerc ilaylydlyt scshlknkfg elfsdfcskv kntiycnvep sesnmrwape |
| | | | | 1021 | fmidtlenpa ahtftytglg kslsenpanr ysfvcnalmh vcvghhdpdr vndiailcae |
| | | | | 1081 | ltgyckslsa ewlgvlkalc cssnngtcgf ndllcnvdvs dlsfhdslat fvailiarqc |
| | | | | 1141 | llledlirca aipsllnaac seqdsepgar ltcrillhlf ktpqlnpcqs dgnkptvgir |
| | | | | 1201 | sscdrhllaa sqnrivdgav favlkavfvl gdaelkgsgf tvtggteelp eeeggggsgg |
| | | | | 1261 | rrqggrnisv etasldvyak yvlrsicqqe wygerclksl cedsndlqdp vlssaqaqrl |
| | | | | 1321 | mqlicyphrl ldnedgenpq rqrikrilqn ldqwtmrqss lelqlmikqt pnnemnslle |
| | | | | 1381 | niakatievf qqsaetgsss gstasnmpss sktkpvlssl ersgvwlvap liaklptsvq |
| | | | | 1441 | ghvlkaagee lekgqhlgss srkerdrqkq ksmsllsqqp flslvltclk gqdeqregll |
| | | | | 1501 | tslysqvhqi vnnwrddqyl ddckpkqlmh ealklrinlv ggmfdtvqrs tqqttewaml |
| | | | | 1561 | lleiiisgtv dmqsnnelft tvldmlsvli ngtlaadmss isqgsmeenk raymnlakkl |
| | | | | 1621 | qkelgerqsd slekvrqllp lpkqtrdvit cepqgslidt kgnkiagfds ifkkeglqvs |
| | | | | 1681 | tkqkispwdl feglkpsapl swgwfgtvry drrvargeeq qrlllyhthl rprprayyle |
| | | | | 1741 | plplppedee ppaptllepe kkapeppktd kpgaappste erkkkstkgk krsqpatkte |
| | | | | 1801 | dygmgpgrsg pygvtvppdl lhhpnpgsit hlnyrqgsig lytqnqplpa ggprvdpyrp |
| | | | | 1861 | vrlpmqklpt rptypgvlpt tmtgvmglep ssyktsvyrq qqpavpqgqr lrqqlqqsqg |
| | | | | 1921 | mlgqssvhqm tpsssyglqt sqgytpyvsh vglqqhtgpa gtmvppsyss qpyqsthpst |
| | | | | 1981 | nptivdptrh lqqrpsgyvh qqaptyghgl tstqrfshqt lqqtpmistm tpmsaqgvqa |
| | | | | 2041 | gvrstailpe qqqqqqqqqq qqqqqqqqqq qqqqqqyhir qqqqqqilrq qqqqqqqqqq |
| | | | | 2101 | qqqqqqqqqq qqqqqhqqqq qqqaappqpq pqsqpqfqrq glqqtqqqqq taalvrqlqq |
| | | | | 2161 | qlsntqpqps tnifgry (SEQ ID NO: 19) |
| Hnrnpk | NP_001305116.1 | heterogeneous nuclear | 440 aa | 1 | meteqpeetf pntetngefg krpaedmeee qafkrsrntd emvelrillq sknagavigk |
| | | ribonucleoprotein K | | 61 | ggknikalrt dynasysvpd ssgperilsi sadietigei lkkiiptlee yqhykgsdfd |
| | | isoform d [Homo | | 121 | celrllihqs laggiigvkg akikelrent qttiklfqec cphstdrvvl iggkpdrvve |
| | | sapiens]. | | 181 | cikiildlis espikgraqp ydpnfydety dyggftmmfd drrgrpvgfp mrgrggfdrm |
| | | | | 241 | ppgrggrpmp psrrdyddms prrgpppppp grggrggsra rnlplppppp prggdlmayd |
| | | | | 301 | rrgrpgdryd gmvgfsadet wdsaidtwsp sewqmayepq ggsgydysya ggrgsygdlg |
| | | | | 361 | gpiittqvti pkdlagsiig kggqrikqir hesgasikid eplegsedri ititgtqdqi |
| | | | | 421 | qnaqyllqns vkqyadvegf (SEQ ID NO: 20) |
| Zfp281 | NP_001268223.1 | zinc finger protein 281 | 859 aa | 1 | mkigsgflsg gggtgssggs gsggggsggg ggggssgrra emeptfpqap aaepppppap |
| | | isoform 2 | | 61 | dmtfkkepaa saaafpsqrt swgflqslvs ikqekpadpe eqqshhhhhh hhygglfaga |
| | | (ZNF281) [Homo | | 121 | eerspglggg eggshgviqd lsilhqhvqq qpaqhhrdvl lssssrtddh hgteepkqdt |
| | | sapiens]. | | 181 | nvkkakrpkp esqgikakrk psasskpslv gdgegailsp sqkphicdhc saafrssyhl |
| | | | | 241 | rrhvlihtge rpfqcsqcsm gfiqkyllqr hekihsrekp fgcdqcsmkf iqkyhmerhk |
| | | | | 301 | rthsgekpyk cdtcqqyfsr tdrllkhrrt cgevivkgat saepgssnht nmgnlavlsq |
| | | | | 361 | gntsssrrkt ksksiaienk eqktgktnes qisnninmqs ysvemptvss sggiigtgid |
| | | | | 421 | elqkrvpkli fkkgsrkntd knylnfvspl pdivgqksls gkpsgslgiv snnsvetigl |
| | | | | 481 | lqstsgkqgq issnyddamq fskkrrylpt assnsafsin vghmvsqqsv iqsagvsvld |
| | | | | 541 | neaplslids salnaeiksc hdksgipdev lqsildqysn ksesqkedpf niaeprvdlh |
| | | | | 601 | tsgehselvq eenlspgtqt psndkasmlq eyskylqqaf ekstnasftl ghgfqfvsls |
| | | | | 661 | splhnhtlfp ekqiyttspl ecgfgqsvts vlpsslpkpp fgmlfgsqpg lylsaldath |
| | | | | 721 | qqltpsqeld dlidsqknle tssafqsssq kltsqkeqkn lesstgfqip sqelasqidp |
| | | | | 781 | qkdieprtty qienfaqafg sqfksgsrvp mtfitnsnge vdhrvrtsys dfsgytnmms |
| | | | | 841 | dvsepcstry ktptsqsyr (SEQ ID NO: 21) |
| Taf51 | NP_055224.1 | TAF5-like RNA | 589 aa | 1 | mkrvrteqiq mayscylkrr qyvdsdgplk qglrlsqtae emaanitvqs esgcanivsa |
| | | polymerase II | | 61 | apcqaepqqy evqfgrlrnf ltdsdsqhsh evmpllyplf vylhlnlvqn spkstvesfy |
| | | p300/CBP-associated | | 121 | srfhgmflqn asqkdvieql qttqtiqdil snfklrafld nkyvvrlqed synylirylq |
| | | factor-associated factor | | 181 | sdnntalckv ltlhihldvq pakrtdyqly asgsssrsen ngleppdmps pilqneaale |
| | | 65 kDa subunit 5L | | 241 | vlqesikrvk dgppslttic fyafynteql lntaeispds kllaagfdns ciklwslrsk |
| | | isoform a [Homo | | 301 | klksephqvd vsrihlacdi leeeddeddn agtemkilrg hcgpvystrf ladssgllsc |
| | | sapiens]. | | 361 | sedmsirywd lgsftntvly qghaypvwdl dispyslyfa sgshdrtarl wsfdrtyplr |
| | | | | 421 | iyaghladvd cvkfhpnsny latgstdktv rlwsaqqgns vrlftghrgp vlslafspng |
| | | | | 481 | kylasagedq rlklwdlasg tlykelrght dnitsltfsp dsgliasasm dnsvrvwdir |
| | | | | 541 | ntycsapadg ssselvgvyt gqmsnvlsvq fmacnillvt gitqenqeh (SEQ ID NO: 22) |
| Ddit3 | NP_001181986.1 | DNA damage- | 169 aa | 1 | maaeslpfsf gtlsswelea wyedlqevls sdenggtyvs ppgneeeesk ifttldpasl |
| | | inducible transcript 3 | | 61 | awlteeepep aevtstsqsp hspdssqssl aqeeeeedqg rtrkrkqsgh sparagkqrm |
| | | protein isoform 2 | | 121 | kekeqenerk vaqlaeener lkqeierltr eveatrrali drmvnlhqa (SEQ ID NO: 23) |
| | | [Homo sapiens]. | | | |
| Zmynd8 | NP_001350670.1 | protein kinase C- | 1186 aa | 1 | mdistrskdp gsaertaqkr kfpspphssn ghspqdtsts pikkkkkpgl lnsnnkeqse |
| | | binding protein 1 | | 61 | lrhgpfyymk qplttdpvdv vpqdgrndfy cwvchregqv lccelcprvy hakclrltse |
| | | isoform t [Homo | | 121 | pegdwfcpec ekitvaecie tqskamtmlt ieqlsyllkf aiqkmkqpgt dafqkpvple |
| | | sapiens]. | | 181 | qhpdyaeyif hpmdlctlek nakkkmygct eafladakwi lhnciiyngg nhkltqiakv |
| | | | | 241 | vikicehemn eievcpecyl aacqkrdnwf cepcsnphpl vwaklkgfpf wpakalrdkd |
| | | | | 301 | gqvdarffgq hdrawvpinn cylmskeipf svkktksifn samqemevyv enirrkfgvf |
| | | | | 361 | nyspfrtpyt pnsqyqmlld ptnpsagtak idkqekvkln fdmtaspkil mskpvlsggt |
| | | | | 421 | grrislsdmp rspmstnssv htgsdveqda ekkatsshfs aseesmdfld kstaspastk |
| | | | | 481 | tgqagslsgs pkpfspqlsa pittktdkts ttgsilnlnl drskaemdlk elsesvqqqs |
| | | | | 541 | tpvplispkr qirsrfqlnl dktiesckaq lgineisedv ytavehsdse dseksdssds |
| | | | | 601 | eyisddeqks knepedtedk egcqmdkeps avkkkpkptn pveikeelks tspasekadp |
| | | | | 661 | gavkdkaspe pekdfsekak psphpikdkl kgkdetdspt vhlgldsdse selvidlged |
| | | | | 721 | hsgregrknk kepkepspkq dvvgktppst tvgshsppet pvltrssaqt saagatatts |
| | | | | 781 | tsstvtvtap apaatgspvk kqrpllpket apavqrvvwn ssskfqtssq kwhmqkmqrq |
| | | | | 841 | qqqqqqqnqq qqpqssqgtr yqtrqavkav qqkeitqsps tstitivtst qssplvtssg |
| | | | | 901 | smstivssvn adlpiatasa dvaadiakyt skmmdaikgt mteiyndlsk nttgstiaei |
| | | | | 961 | rrlrieiekl qwlhqqelse mkhnleltma emrqsleqer drliaevkkq lelekqqavd |
| | | | | 1021 | etkkkqwcan ckkeaifycc wntsycdypc qqahwpehmk sctqsatapq qeadaevnte |
| | | | | 1081 | tlnkssqgss sstqsapset asaskekets aekskesgst ldlsgsretp ssillgsnqg |
| | | | | 1141 | sdhsrsnkss wsssdekrgs trsdhntsts tksllpkesr ldtfwd (SEQ ID NO: 24) |
| Med14 | NP_004220.2 | mediator of RNA | 1454 aa | 1 | mapvqlenhq lvppgggggg sggppsapap pppgaavaaa aaaaaspgyr lstliefllh |
| | | polymerase II | | 61 | rayselmvlt dllprksdve rkieivqfas rtrqlfvrll alvkwannag kvekcamiss |
| | | transcription subunit 14 | | 121 | fldqqailfv dtadrlasla rdalvharlp sfaipyaidv lttgsyprlp tcirdkiipp |
| | | [Homo sapiens]. | | 181 | dpitkiekqa tlhqlnqilr hrlyttdlpp qlanitvang rvkfrvegef eatltvmgdd |
| | | | | 241 | pdvpwrllkl eilvedketg dgralvhsmq isfihqlvqs rlfadekplq dmynclhsfc |
| | | | | 301 | lslqlevlhs qtlmlirerw gdlvqveryh agkelslsvw nqqvlgrktg tasvhkvtik |
| | | | | 361 | idendvskpl qifhdpplpa sdsklveram kidhlsiekl lidsvharah qklqelkail |
| | | | | 421 | rgfnanenss ietalpalvv pilepcgnse clhifvdlhs gmfqlmlygl dqatlddmek |
| | | | | 481 | svnddmkrii pwiqqlkfwl gqqrckqsik hlptissetl qlsnysthpi gnlsknklfi |
| | | | | 541 | kltrlpqyyi vvemlevpnk ptqlsykyyf msvnaadred spamalllqq fkeniqdlvf |
| | | | | 601 | rtktgkqtrt nakrklsddp cpveskktkr agemcafnkv lahfvamcdt nmpfvglrle |
| | | | | 661 | lsnleiphqg vqvegdgfsh airllkippc kgiteetqka ldrslldctf rlqgninrtw |
| | | | | 721 | vaelvfancp lngtstreqg psrhvyltye nllsepvggr kvvemflndw nsiarlyecv |
| | | | | 781 | lefarslpdi pahlnifsev rvynyrklil cygttkgssi siqwnsihqk fhislgtvgp |
| | | | | 841 | nsgcsnchnt ilhqlqemfn ktpnvvqllq vlfdtqapin ainklptvpm lgltqrtnta |
| | | | | 901 | yqcfsilpqs sthirlafrn mycidiycrs rgvvairdga yslfdnsklv egfypapglk |
| | | | | 961 | tflnmfvdsn qdarrrsvne ddnppspigg dmmdslisql qpppqqqpfp kqpgtsgayp |
| | | | | 1021 | ltspptsyhs tvnqspsmmh tqspgnlhaa sspsgalrap spasfvptpp psshgisigp |
| | | | | 1081 | gasfasphgt ldpsspytmv spsgragnwp gspqvsgpsp aarmpgmspa npslhspvpd |
| | | | | 1141 | ashspragts sqtmptnmpp prklpqrswa asiptilths alnilllpsp tpglvpglag |
| | | | | 1201 | sylcsplerf lgsvimrrhl qriiqqetlq linsnepgvi mfktdalkcr valspktnqt |
| | | | | 1261 | lqlkvtpena gqwkpdelqv lekffetrva gppfkantli aftkllgapt hilrdcvhim |
| | | | | 1321 | klelfpdqat qlkwnvqfcl tippsappia ppgtpavvlk skmlfflqlt qktsvppqep |
| | | | | 1381 | vsiivpiiyd masgttqqad iprqqnssva apmmvsnilk rfaemnpprq gectifaavr |
| | | | | 1441 | dlmanitlpp ggrp (SEQ ID NO: 25) |
| Rad21 | NP_006256.1 | double-strand-break | 631 aa | 1 | mfyahfvlsk rgplakiwla ahwdkkltka hvfecnless vesiispkvk malrtsghll |
| | | repair protein rad21 | | 61 | lgvvriyhrk akylladcne afikikmafr pgvvdlpeen reaaynaitl peefhdfdqp |
| | | homolog [Homo | | 121 | lpdlddidva qqfslnqsry eeitmreevg nisilqendf gdfgmddrei mregsafedd |
| | | sapiens]. | | 181 | dmlvstttsn llleseqsts nlnekinhle yedqykddnf gegndggild dklisnndgg |
| | | | | 241 | ifddppalse agvmlpeqpa hddmdeddnv smggpdspds vdpvepmptm tdqttivpne |
| | | | | 301 | eeafalepid itvketkakr krklivdsvk eldsktiraq lsdysdivtt ldlapptkkl |
| | | | | 361 | mmwketggve klfslpaqpl wnnfilklft rcltplyped lrkrrkggea dnldeflkef |
| | | | | 421 | enpevpredq qqqhqqrdvi depiieepsr lqesvmeasr tnidesampp pppqgvkrka |
| | | | | 481 | gqidpepvmp pqqveqmeip pvelppeepp nicqlipele llpekekeke kekeddeeee |
| | | | | 541 | dedasggdqd qeerrwnkrt qqmlhglqra laktgaesis llelcrntnr kqaaakfysf |
| | | | | 601 | lvlkkqqaie ltqeepysdi iatpgprfhi l (SEQ ID NO: 26) |
| Dmapl | NP_001029196.1 | DNA methyltransferase | 467 aa | 1 | matgadvrdi lelggpegda asgtiskkdi inpdkkkskk ssetltfkrp egmhrevyal |
| | | 1-associated protein 1 | | 61 | lysdkkdapp llpsdtgqgy rtvkaklgsk kvrpwkwmpf tnparkdgam ffhwrraaee |
| | | [Homo sapiens]. | | | |
| | | | | 121 | gkdypfarfn ktvqvpvyse qeyqlylhdd awtkaetdhl fdlsrrfdlr fvvihdrydh |
| | | | | 181 | qqfkkrsved lkeryyhica klanvravpg tdlkipvfda gherrrkeql erlynrtpeq |
| | | | | 241 | vaeeeyllqe lrkiearkke rekrsqdlqk litaadttae qrrterkapk kklpqkkeae |
| | | | | 301 | kpavpetagi kfpdfksagv tlrsqrmklp ssvgqkkika leqmllelgv elsptpteel |
| | | | | 361 | vhmfnelrsd lvllyelkqa canceyelqm lrhrhealar agvlggpatp asgpgpasae |
| | | | | 421 | pavtepglgp dpkdtiidvv gapltpnsrk rresasssss vkkakkp (SEQ ID NO: 27) |
| Med11 | NP_001291929.1 | mediator of RNA | 85 aa | 1 | matyslaner lralediere igailqnagt vilelskekt nerlldrqaa aftasvqhve |
| | | polymerase II | | 61 | aelsaqiryl tqlpdgltns nsgkk (SEQ ID NO: 28) |
| | | transcription subunit 11 | | | |
| | | isoform b | | | |
| Zkscan3 | NP_001229824.1 | zinc finger protein with | 390 aa | 1 | malltpapgs qssqfqlmka llkhesvgsq plqdrvlqvp vlahggccre dkvvasrltp |
| | | KRAB and SCAN | | 61 | esqgllkved valtltpewt qqdssqgnlc rdekqenhgs lvslgdekqt ksrdlppaee |
| | | domains 3 isoform 2 | | 121 | lpekehgkis chlrediaqi ptcaeageqe grlqrkqkna tggrrhiche cgksfaqssg |
| | | [Homo sapiens]. | | 181 | lskhrrihtg ekpyeceecg kafigssalv ihqrvhtgek pyeceecgka fshssdlikh |
| | | | | 241 | qrthtgekpy ecddcgktfs qscsllehhr ihtgekpyqc smcgkafrrs shllrhqrih |
| | | | | 301 | tgdknvqepe qgeawksrme sqlenvetpm sykcnecers ftqntglieh qkihtgekpy |
| | | | | 361 | qcnacgkgft risylvqhqr shvgknilsq (SEQ ID NO: 29) |
| Foxp1 | NP_001336267.1 | forkhead box protein | 677 aa | 1 | mmqesgtetk sngsaiqngs ggsnhllecg glregrsnge tpavdigaad lahaqqqqqq |
| | | P1 isoform a [Homo | | 61 | alqvarqlll qqqqqqqvsg lkspkrndkq palqvpvsva mmtpqvitpq qmqqilqqqv |
| | | sapiens]. | | 121 | lspqqlqvll qqqqalmlqq qqlqefykkq qeqlqlqllq qqhagkqpke qqqvatqqla |
| | | | | 181 | fqqqllqmqq lqqqhllslq rqglltiqpg qpalplqpla qgmiptelqq lwkevtsaht |
| | | | | 241 | aeettgnnhs sldltttcvs ssapsktsli mnphastngq lsvhtpkres lsheehphsh |
| | | | | 301 | plyghgvckw pgceavcedf qsflkhlnse halddrstaq crvqmqvvqq lelqlakdke |
| | | | | 361 | rlqammthlh vkstepkaap qpinlvssvt lsksaseasp qslphtpttp tapltpvtqg |
| | | | | 421 | psvitttsmh tvgpirrrys dkynvpissa diaqnqefyk naevrppfty aslirqaile |
| | | | | 481 | spekqltlne iynwftrmfa yfrrnaatwk navrhnlslh kcfvrvenvk gavwtvdeve |
| | | | | 541 | fqkrrpqkis gnpsliknmq sshayctpin aalqasmaen siplyttasm gnptlgnlas |
| | | | | 601 | aireelngam ehtnsnesds spgrspmqav hpvhvkeepl dpeeaegpls lvttanhspd |
| | | | | 661 | fdhdrdyede pvnedme (SEQ ID NO: 30) |
| Stat5b | NP_036580.2 | signal transducer and | 787 aa | 1 | maywiqaqql qgealhqmqa lygqhfpiev rhylsqwies qawdsvdldn pqenikatql |
| | | activator of | | 61 | leglvqelqk kaehqvgedg fllkiklghy atqlqntydr cpmelvrcir hilyneqrlv |
| | | transcription 5B | | 121 | reanngsspa gsladamsqk hlqinqtfee lrlvtqdten elkklqqtqe yfiiqyqesl |
| | | [Homo sapiens]. | | 181 | riqaqfgpla qlspqerlsr etalqqkqvs leawlqreaq tlqqyrvela ekhqktlqll |
| | | | | 241 | rkqqtiildd eliqwkrrqq lagnggppeg sldvlqswce klaeiiwqnr qqirraehlc |
| | | | | 301 | qqlpipgpve emlaevnati tdiisalvts tfiiekqppq vlktqtkfaa tvrllvggkl |
| | | | | 361 | nvhmnppqvk atiiseqqak sllknentrn dysgeilnnc cvmeyhqatg tlsahfrnms |
| | | | | 421 | lkrikrsdrr gaesvteekf tilfesqfsv ggnelvfqvk tlslpvvviv hgsqdnnata |
| | | | | 481 | tvlwdnafae pgrvpfavpd kvlwpqlcea lnmkfkaevq snrgltkenl vflaqklfnn |
| | | | | 541 | ssshledysg lsyswsqfnr enlpgrnytf wqwfdgvmev lkkhlkphwn dgailgfvnk |
| | | | | 601 | qqandllink pdgtfllrfs dseiggitia wkfdsqermf wnlmpfttrd fsirsladrl |
| | | | | 661 | gdlnyliyvf pdrpkdevys kyytpvpces atakavdgyv kpqikqvvpe fvnasadagg |
| | | | | 721 | gsatymdqap spavcpqahy nmypqnpdsv ldtdgdfdle dtmdvarrve ellgrpmdsq |
| | | | | 781 | wiphaqs (SEQ ID NO: 31) |

Stability of Treg cells may be assessed using FACS markers. Some of the FACS markers used are canonical Treg cell signature proteins. For example, with a specific gene knocked-out or inhibited in Treg cells, if these modified cells display a gain or maintenance of Treg cell canonical markers, such as FOXP3, CTLA4, CD25, IL-10, and/or IKZF2, this may indicate the Treg cells are more stabilized. In some embodiments, a loss of Treg cell canonical markers and/or gain of pro-inflammatory markers (e.g., IL-17a, IL-4, IFNγ, and IL-2) may indicate that the Treg cells are destabilized. In another example, with overexpression of a specific nuclear factor in Treg cells, if these modified cells display a gain or maintenance of Treg cell canonical markers, such as FOXP3, CTLA4, CD25, IL-10, and/or IKZF2, this may indicate the Treg cells are more stabilized. In some embodiments, with overexpression of a specific nuclear factor in Treg cells, if these modified cells display a loss of Treg cell canonical markers and/or gain of pro-inflammatory markers (e.g., IL-17a, IL-4, IFNγ, and IL-2), this may indicate that the Treg cells are destabilized. For methods of detecting and enriching for Tregs, see, for example, International Patent Application Publication No. WO2007140472.

In some embodiments of the methods described herein, inhibiting the expression of a nuclear factor set forth in Table 1 or Table 2 may comprise reducing expression of the nuclear factor or reducing expression of a polynucleotide, for example, an mRNA, encoding the nuclear factor in the Treg cell. In some embodiments expression of one or more nuclear factor s set forth in Table 1 or Table 2 is inhibited in the Treg cell. As described in detail further herein, one or more available methods may be used to inhibit the expression of one or more nuclear factors set forth in Table 1 or Table 2.

In some embodiments of the methods described herein, overexpressing a nuclear factor set forth in Table 1 or a nuclear factor set forth in Table 2 may comprise introducing a polynucleotide encoding the nuclear factor into the Treg cell. In other embodiments of the methods described herein, overexpressing a nuclear factor set forth in Table 1 or a nuclear factor set forth in Table 2 may comprise introducing an agent that induces expression of the endogenous gene encoding the nuclear factor in the Treg cell. For example, RNA activation, where short double-stranded RNAs induce endogenous gene expression by targeting promoter sequences, can be used to induce endogenous gene expression (See, for example, Wang et al. “Inducing gene expression by targeting promoter sequences using small activating RNAs,” J. Biol. Methods 2(1): e14 (2015). In another example, artificial transcription factors containing zinc-finger binding domains can be used to activate or repress expression of endogenous genes. See, for example, Dent et al., “Regulation of endogenous gene expressing using small molecule-controlled engineered zinc-finger protein transcription factors,” Gene Ther. 14(18): 1362-9 (2007).

In some embodiments, inhibiting expression may comprise contacting a polynucleotide encoding the nuclear factor, with a target nuclease, a guide RNA (gRNA), an siRNA, an antisense RNA, microRNA (miRNA), or short hairpin RNA (shRNA). In particular embodiments, if a gRNA and a target nuclease (e.g., Cas9) are used to inhibit the expression of a polynucleotide encoding a human nuclear factor set forth in Table 1 or Table 2, the gRNA may comprise a sequence set forth in Table 3, a sequence complementary to a sequence set forth in Table 3, or a portion thereof. Table 3 provides the Gene ID number, Genbank Accession No. for mRNA, genomic sequence, position in the genome after nuclease cutting, sgRNA target sequence, target context sequence, PAM sequence, and the exon targeted by the sgRNA for each nuclear factor set forth in Tables 1 and 2. ZNF281 is the human homolog of mouse Zfp281.

TABLE 3
gRNA target sequences and related information for targeting nuclear factors

| | | | | Position | | | | | |
|---|---|---|---|---|---|---|---|---|---|
| Target | Target | | | of Base | | | | | |
| Gene | Gene | Target | Genomic | After | | | | | |
| ID | Symbol | Transcript | Sequence | Cut (1-based) | Strand | sgRNA Target Sequence | Target Context Sequence | PAM Seq. | Exon No. |
| 6667 | SP1 | NM_001251825.1 | NC_000012.12 | 53382598 | sense | CAACAGATTATCACAAATC | AAACCAACAGATTATCACAAATCGAGGAAG | AGG | 3 |
| | | | | | | G (SEQ ID NO: 32) | (SEQ ID NO: 152) | | |
| 6667 | SP1 | NM_001251825.1 | NC_000012.12 | 53383311 | sense | CATCATCCGGACACCAACA | CCATCATCATCCGGACACCAACAGTGGGGC | TGG | 3 |
| | | | | | | G(SEQ ID NO: 33) | (SEQ ID NO: 153) | | |
| 6667 | SP1 | NM_001251825.1 | NC_000012.12 | 53382717 | sense | GTATGTGACCAATGTACCA | CTCAGTATGTGACCAATGTACCAGTGGCCC | TGG | 3 |
| | | | | | | G (SEQ ID NO: 34) | (SEQ ID NO: 154) | | |
| 6667 | SP1 | NM_001251825.1 | NC_000012.12 | 53382986 | sense | TTACTACCAGTGGATCATC | AACTTTACTACCAGTGGATCATCAGGGACC | GGG | 3 |
| | | | | | | A (SEQ ID NO: 35) | (SEQ ID NO: 155) | | |
| 56254 | RNF20 | NM_019592.6 | NC_000009.12 | 101547487 | sense | ACTTCGGCAAGACTTTGAG | AGAAACTTCGGCAAGACTTTGAGGAGGTCA | AGG | 9 |
| | | | | | | G (SEQ ID NO: 36) | (SEQ ID NO: 156) | | |
| 56254 | RNF20 | NM_019592.6 | NC_000009.12 | 101544881 | sense | GCATCGCACCATGTCTCAG | AAAAGCATCGCACCATGTCTCAGGAGGTAC | AGG | 6 |
| | | | | | | G (SEQ ID NO: 37) | (SEQ ID NO: 157) | | |
| 56254 | RNF20 | NM_019592.6 | NC_000009.12 | 101552394 | antisense | GGAGGGCACTACCACTACG | TGCAGGAGGGCACTACCACTACGCAGGCGT | AGG | 13 |
| | | | | | | C (SEQ ID NO: 38) | (SEQ ID NO: 158) | | |
| 56254 | RNF20 | NM_019592.6 | NC_000009.12 | 101540342 | antisense | TCGGTTGACAATCAATAGT | AGTATCGGTTGACAATCAATAGTGAGGCAT | AGG | 3 |
| | | | | | | G (SEQ ID NO: 39) | (SEQ ID NO: 159) | | |
| 64864 | RFX7 | NM_022841.5 | NC_000015.10 | 56098123 | antisense | ACAACGATACCAATAGGTT | TGCCACAACGATACCAATAGGTTGAGGAGA | AGG | 8 |
| | | | | | | G (SEQ ID NO: 40) | (SEQ ID NO: 160) | | |
| 64864 | RFX7 | NM_022841.5 | NC_000015.10 | 56095516 | antisense | AGCTGAATCACTGATAACA | CCAAAGCTGAATCACTGATAACAAGGGCAG | GGG | 9 |
| | | | | | | A (SEQ ID NO: 41) | (SEQ ID NO: 161) | | |
| 64864 | RFX7 | NM_022841.5 | NC_000015.10 | 56142833 | sense | CTGGATTCGGAATACCCTA | TTTCCTGGATTCGGAATACCCTAGAGGAAC | AGG | 4 |
| | | | | | | G (SEQ ID NO: 42) | (SEQ ID NO: 162) | | |
| 64864 | RFX7 | NM_022841.5 | NC_000015.10 | 56101446 | antisense | GAAGCGGGCTAATTCCAAG | CAAGGAAGCGGGCTAATTCCAAGACGGTGT | CGG | 7 |
| | | | | | | A (SEQ ID NO: 43) | (SEQ ID NO: 163) | | |
| 6722 | SRF | NM_003131.3 | NC_000006.12 | 43175724 | antisense | AGGTTGGTGACTGTGAACG | CGGCAGGTTGGTGACTGTGAACGCCGGCTT | CGG | 3 |
| | | | | | | C (SEQ ID NO: 44) | (SEQ ID NO: 164) | | |
| 6722 | SRF | NM_003131.3 | NC_000006.12 | 43172119 | sense | AGTTCATCGACAACAAGCT | ATGGAGTTCATCGACAACAAGCTGCGGCGC | CGG | 1 |
| | | | | | | G (SEQ ID NO: 45) | (SEQ ID NO: 165) | | |
| 6722 | SRF | NM_003131.3 | NC_000006.12 | 43175844 | antisense | GGGCTGACACTAGCAGAC | ACTGGGGCTGACACTAGCAGACACTGGTGC | TGG | 3 |
| | | | | | | AC (SEQ ID NO: 46) | (SEQ ID NO: 166) | | |
| 6722 | SRF | NM_003131.3 | NC_000006.12 | 43174015 | antisense | TCTGTTGTGGGGTCTGAAC | CTGGTCTGTTGTGGGGTCTGAACGGGGTGG | GGG | 2 |
| | | | | | | G (SEQ ID NO: 47) | (SEQ ID NO: 167) | | |
| 55250 | ELP2 | NM_018255.2 | NC_000018.10 | 36156467 | antisense | AATTTCATGCCAAGTCACC | TTGCAATTTCATGCCAAGTCACCTGGGTAA | GGG | 13 |
| | | | | | | T (SEQ ID NO: 48) | (SEQ ID NO: 168) | | |
| 55250 | ELP2 | NM_018255.2 | NC_000018.10 | 36141150 | sense | CCAGTACCAATATTAGCAT | TCCCCCAGTACCAATATTAGCATGTGGCAA | TGG | 6 |
| | | | | | | G (SEQ ID NO: 49) | (SEQ ID NO: 169) | | |
| 55250 | ELP2 | NM_018255.2 | NC_000018.10 | 36146255 | sense | GTTATTGTACAGGTTCGAG | GTCTGTTATTGTACAGGTTCGAGTAGGTGA | AGG | 11 |
| | | | | | | T (SEQ ID NO: 50) | (SEQ ID NO: 170) | | |
| 55250 | ELP2 | NM_018255.2 | NC_000018.10 | 36136355 | sense | TGATAATCAAGTGATTCAC | GATCTGATAATCAAGTGATTCACTGGGAAA | GGG | 3 |
| | | | | | | T (SEQ ID NO: 51) | (SEQ ID NO: 171) | | |
| 64324 | NSD1 | NM_022455.4 | NC_000005.10 | 177209972 | sense | AAGCACATAAAGATGAAC | TTTGAAGCACATAAAGATGAACGGAGGGGA | AGG | 5 |
| | | | | | | GG (SEQ ID NO: 52) | (SEQ ID NO: 172) | | |
| 64324 | NSD1 | NM_022455.4 | NC_000005.10 | 177238503 | sense | GAATTGCTAGTTAAAACGC | TGAGGAATTGCTAGTTAAAACGCCAGGTAA | AGG | 7 |
| | | | | | | C (SEQ ID NO: 53) | (SEQ ID NO: 173) | | |
| 64324 | NSD1 | NM_022455.4 | NC_000005.10 | 177204150 | sense | GCCCTATCGGCAGTACTAC | GGAGGCCCTATCGGCAGTACTACGTGGAGG | TGG | 4 |
| | | | | | | G (SEQ ID NO: 54) | (SEQ ID NO: 174) | | |
| 64324 | NSD1 | NM_022455.4 | NC_000005.10 | 177211164 | sense | TATGCATGATAGTAAGACG | AAGATATGCATGATAGTAAGACGAAGGAGC | AGG | 5 |
| | | | | | | A (SEQ ID NO: 55) | (SEQ ID NO: 175) | | |
| 6598 | SMARCB1 | NM_003073.3 | NC_000022.11 | 23791773 | antisense | GAGAACCTCGGAACATAC | TACAGAGAACCTCGGAACATACGGAGGTAG | AGG | 2 |
| | | | | | | GG (SEQ ID NO: 56) | (SEQ ID NO: 176) | | |
| 6598 | SMARCB1 | NM_003073.3 | NC_000022.11 | 23816887 | sense | GCAGATCGAGTCCTACCCC | GACAGCAGATCGAGTCCTACCCCACGGACA | CGG | 6 |
| | | | | | | A (SEQ ID NO: 57) | (SEQ ID NO: 177) | | |
| 6598 | SMARCB1 | NM_003073.3 | NC_000022.11 | 23801049 | antisense | TCTTCTTGTCTCGGCCCATG | GTTCTCTTCTTGTCTCGGCCCATGCGGTTC | CGG | 4 |
| | | | | | | (SEQ ID NO: 58) | (SEQ ID NO: 178) | | |
| 6598 | SMARCB1 | NM_003073.3 | NC_000022.11 | 23803342 | sense | TGAGAACGCATCTCAGCCC | TCCATGAGAACGCATCTCAGCCCGAGGTGC | AGG | 5 |
| | | | | | | G (SEQ ID NO: 59) | (SEQ ID NO: 179) | | |
| 10365 | KLF2 | NM_016270.2 | NC_000019.10 | 16325729 | antisense | AAACCAGGGCCACCGAAA | GCCGAAACCAGGGCCACCGAAAGGCGGCGG | CGG | 2 |
| | | | | | | GG (SEQ ID NO: 60) | (SEQ ID NO: 180) | | |
| 10365 | KLF2 | NM_016270.2 | NC_000019.10 | 16325576 | antisense | CCCTCGCGCTTGAGGCCGC | GGCGCCCTCGCGCTTGAGGCCGCGCGGTCC | CGG | 2 |
| | | | | | | G (SEQ ID NO: 61) | (SEQ ID NO: 181) | | |
| 10365 | KLF2 | NM_016270.2 | NC_000019.10 | 16325811 | sense | CTTCGGTCTCTTCGACGAC | CAGCCTTCGGTCTCTTCGACGACGCGGCCG | CGG | 2 |
| | | | | | | G (SEQ ID NO: 62) | (SEQ ID NO: 182) | | |
| 10365 | KLF2 | NM_016270.2 | NC_000019.10 | 16325354 | antisense | TCGGGGTAATAGAACGCA | GGGTTCGGGGTAATAGAACGCAGGCGGCGG | CGG | 2 |
| | | | | | | GG (SEQ ID NO: 63) | (SEQ ID NO: 183) | | |
| 10664 | CTCF | NM_006565.3 | NC_000016.10 | 67612001 | antisense | CGATCCAAATTTGAACGCC | GTGACGATCCAAATTTGAACGCCGTGGACA | TGG | 4 |
| | | | | | | G (SEQ ID NO: 64) | (SEQ ID NO: 184) | | |
| 10664 | CTCF | NM_006565.3 | NC_000016.10 | 67611476 | sense | GAGCAAACTGCGTTATACA | AAAAGAGCAAACTGCGTTATACAGAGGAGG | AGG | 3 |
| | | | | | | G (SEQ ID NO: 65) | (SEQ ID NO: 185) | | |
| 10664 | CTCF | NM_006565.3 | NC_000016.10 | 67610967 | sense | TTACCCCAGAACCAGACGG | CCACTTACCCCAGAACCAGACGGATGGGGG | TGG | 3 |
| | | | | | | A (SEQ ID NO: 66) | (SEQ ID NO: 186) | | |
| 10664 | CTCF | NM_006565.3 | NC_000016.10 | 67620773 | sense | TTTGTGCAGTTATGCCAGC | GCAGTTTGTGCAGTTATGCCAGCAGGGACA | GGG | 6 |
| | | | | | | A (SEQ ID NO: 67) | (SEQ ID NO: 187) | | |
| 6304 | SATB1 | NM_002971.4 | NC_000003.12 | 18415117 | antisense | ATGCTAAGTACCTGTGAAA | TTCTATGCTAAGTACCTGTGAAAGGGGGCA | GGG | 5 |
| | | | | | | G (SEQ ID NO: 68) | (SEQ ID NO: 188) | | |
| 6304 | SATB1 | NM_002971.4 | NC_000003.12 | 18417016 | sense | CATTGAATATGATTGCAAG | ACGCCATTGAATATGATTGCAAGGAGGAGC | AGG | 3 |
| | | | | | | G (SEQ ID NO: 69) | (SEQ ID NO: 189) | | |
| 6304 | SATB1 | NM_002971.4 | NC_000003.12 | 18394751 | antisense | TAGGTGTTGATACGAGCCC | CTGATAGGTGTTGATACGAGCCCAGGGTGC | GGG | 7 |
| | | | | | | A (SEQ ID NO: 70) | (SEQ ID NO: 190) | | |
| 6304 | SATB1 | NM_002971.4 | NC_000003.12 | 18394610 | antisense | TATTCATAGATCTACTGAC | GGCTTATTCATAGATCTACTGACAGGGGGA | GGG | 7 |
| | | | | | | A (SEQ ID NO: 71) | (SEQ ID NO: 191) | | |
| 50943 | FOXP3 | NM_014009.3 | NC_000023.11 | 49254057 | sense | ACCCAGGCATCATCCGACA | CCTCACCCAGGCATCATCCGACAAGGGCTC | GGG | 9 |
| | | | | | | A (SEQ ID NO: 72) | (SEQ ID NO: 192) | | |
| 50943 | FOXP3 | NM_014009.3 | NC_000023.11 | 49257007 | sense | CCCACCCACAGGGATCAAC | TGTCCCCACCCACAGGGATCAACGTGGCCA | TGG | 5 |
| | | | | | | G (SEQ ID NO: 73) | (SEQ ID NO: 193) | | |
| 50943 | FOXP3 | NM_014009.3 | NC_000023.11 | 49255795 | sense | CCTACTTAGGCACTGCCAG | TCTCCCTACTTAGGCACTGCCAGGCGGACC | CGG | 7 |
| | | | | | | G (SEQ ID NO: 74) | (SEQ ID NO: 194) | | |
| 50943 | FOXP3 | NM_014009.3 | NC_000023.11 | 49257751 | antisense | GAGGGTGCCACCATGACTA | CCCGGAGGGTGCCACCATGACTAGGGGCAG | GGG | 3 |
| | | | | | | G (SEQ ID NO: 75) | (SEQ ID NO: 195) | | |
| 23326 | USP22 | NM_015276.1 | NC_000017.11 | 21015837 | sense | ACCTGGTGTGGACCCACGC | CTGCACCTGGTGTGGACCCACGCGAGGCAC | AGG | 6 |
| | | | | | | G (SEQ ID NO: 76) | (SEQ ID NO: 196) | | |
| 23326 | USP22 | NM_015276.1 | NC_000017.11 | 21019085 | sense | CCTCGAACTGCACCATAGG | ATCACCTCGAACTGCACCATAGGTGGGTGG | GGG | 4 |
| | | | | | | T (SEQ ID NO: 77) | (SEQ ID NO: 197) | | |
| 23326 | USP22 | NM_015276.1 | NC_000017.11 | 21021211 | sense | GCCATTGATCTGATGTACG | CTCAGCCATTGATCTGATGTACGGAGGCAT | AGG | 3 |
| | | | | | | G (SEQ ID NO: 78) | (SEQ ID NO: 198) | | |
| 23326 | USP22 | NM_015276.1 | NC_000017.11 | 21018000 | antisense | TGGGGCTCTGCATCTCACA | GAGCTGGGGCTCTGCATCTCACAGCGGTGC | CGG | 5 |
| | | | | | | G (SEQ ID NO: 79) | (SEQ ID NO: 199) | | |
| 865 | CBFB | NM_001755.2 | NC_000016.10 | 67036720 | antisense | AAGTCGACATACTCTCGGC | TTCTAAGTCGACATACTCTCGGCTAGGTGT | AGG | 3 |
| | | | | | | T (SEQ ID NO: 80) | (SEQ ID NO: 200) | | |
| 865 | CBFB | NM_001755.2 | NC_000016.10 | 67029479 | antisense | CCTGCCTCACCTCACACTC | CCCGCCTGCCTCACCTCACACTCGCGGCTC | CGG | 1 |
| | | | | | | G (SEQ ID NO: 81) | (SEQ ID NO: 201) | | |
| 865 | CBFB | NM_001755.2 | NC_000016.10 | 67029807 | antisense | GCCGACTTACGATTTCCGA | GCCAGCCGACTTACGATTTCCGAGCGGCCG | CGG | 2 |
| | | | | | | G (SEQ ID NO: 82) | (SEQ ID NO: 202) | | |
| 865 | CBFB | NM_001755.2 | NC_000016.10 | 67066729 | sense | GGAGTCTGTGTTATCTGGA | GAATGGAGTCTGTGTTATCTGGAAAGGCTG | AGG | 4 |
| | | | | | | A (SEQ ID NO: 83) | (SEQ ID NO: 203) | | |
| 861 | RUNX1 | NM_001754.4 | NC_000021.9 | 34880580 | antisense | CACTTCGACCGACAAACCT | CTTCCACTTCGACCGACAAACCTGAGGTCA | AGG | 5 |
| | | | | | | G (SEQ ID NO: 84) | (SEQ ID NO: 204) | | |
| 861 | RUNX1 | NM_001754.4 | NC_000021.9 | 34799436 | antisense | CTGATCGTAGGACCACGGT | AGGACTGATCGTAGGACCACGGTGGGGATG | GGG | 8 |
| | | | | | | G (SEQ ID NO: 85) | (SEQ ID NO: 205) | | |
| 861 | RUNX1 | NM_001754.4 | NC_000021.9 | 34834458 | antisense | GGCAGTGGAGTGGTTCAGG | TAAAGGCAGTGGAGTGGTTCAGGGAGGCAC | AGG | 7 |
| | | | | | | G (SEQ ID NO: 86) | (SEQ ID NO: 206) | | |
| 861 | RUNX1 | NM_001754.4 | NC_000021.9 | 34834570 | sense | TAGATGATCAGACCAAGCC | AAACTAGATGATCAGACCAAGCCCGGGAGC | GGG | 7 |
| | | | | | | C (SEQ ID NO: 87) | (SEQ ID NO: 207) | | |
| 4609 | MYC | NM_002467.4 | NC_000008.11 | 127738837 | sense | AGAGTGCATCGACCCCTCG | CCTCAGAGTGCATCGACCCCTCGGTGGTCT | TGG | 2 |
| | | | | | | G (SEQ ID NO: 88) | (SEQ ID NO: 208) | | |
| 4609 | MYC | NM_002467.4 | NC_000008.11 | 127738942 | antisense | CTGCGGGGAGGACTCCGTC | TGCCCTGCGGGGAGGACTCCGTCGAGGAGA | AGG | 2 |
| | | | | | | G (SEQ ID NO: 89) | (SEQ ID NO: 209) | | |
| 4609 | MYC | NM_002467.4 | NC_000008.11 | 127738523 | sense | CTTCGGGGAGACAACGAC | CTCCCTTCGGGGAGACAACGACGGCGGTGG | CGG | 2 |
| | | | | | | GG (SEQ ID NO: 90) | (SEQ ID NO: 210) | | |
| 4609 | MYC | NM_002467.4 | NC_000008.11 | 127738307 | antisense | GCTGCACCGAGTCGTAGTC | TACGGCTGCACCGAGTCGTAGTCGAGGTCA | AGG | 2 |
| | | | | | | G (SEQ ID NO: 91) | (SEQ ID NO: 211) | | |
| 6760 | SS18 | NM_001007559.1 | NC_000018.10 | 260526860 | sense | AATCAGATGACAATGAGTC | ACAGAATCAGATGACAATGAGTCAGGGACA | GGG | 5 |
| | | | | | | A (SEQ ID NO: 92) | (SEQ ID NO: 212) | | |
| 6760 | SS18 | NM_001007559.1 | NC_000018.10 | 26039408 | sense | CAATACAATATGCCACAGG | TCAGCAATACAATATGCCACAGGGAGGCGG | AGG | 6 |
| | | | | | | G (SEQ ID NO: 93) | (SEQ ID NO: 213) | | |
| 6760 | SS18 | NM_001007559.1 | NC_000018.10 | 26052827 | sense | CCTAACCATATGCCTATGC | AGGGCCTAACCATATGCCTATGCAGGGACC | GGG | 5 |
| | | | | | | A (SEQ ID NO: 94) | (SEQ ID NO: 214) | | |
| 6760 | SS18 | NM_001007559.1 | NC_000018.10 | 26057677 | antisense | GGCATGTTGTGAGAGCGTG | TGAAGGCATGTTGTGAGAGCGTGGAGGTGG | AGG | 4 |
| | | | | | | G (SEQ ID NO: 95) | (SEQ ID NO: 215) | | |
| 90390 | MED30 | NM_080651.3 | NC_000008.11 | 117528690 | sense | ACACTGGAACATATCAAGA | TACCACACTGGAACATATCAAGACCGGTTA | CGG | 2 |
| | | | | | | C (SEQ ID NO: 96) | (SEQ ID NO: 216) | | |
| 90390 | MED30 | NM_080651.3 | NC_000008.11 | 117528779 | sense | GACAAATGCAATGAAAAC | ATATGACAAATGCAATGAAAACTGTGGTGG | TGG | 2 |
| | | | | | | TG (SEQ ID NO: 97) | (SEQ ID NO: 217) | | |
| 90390 | MED30 | NM_080651.3 | NC_000008.11 | 117521019 | sense | GGACATCGTGTACCGCACC | TGCAGGACATCGTGTACCGCACCATGGAGA | TGG | 1 |
| | | | | | | A (SEQ ID NO: 98) | (SEQ ID NO: 218) | | |
| 90390 | MED30 | NM_080651.3 | NC_000008.11 | 117520962 | sense | GGCCGCCCGGGAAGTCAA | AGCAGGCCGCCCGGGAAGTCAACACGGCGT | CGG | 1 |
| | | | | | | CA (SEQ ID NO: 99) | (SEQ ID NO: 219) | | |
| 56970 | ATXN7L3 | NM_001098833.1 | NC_000017.11 | 44197610 | sense | CACGGACCCTGATAGCATG | ACGACACGGACCCTGATAGCATGAAGGATT | AGG | 2 |
| | | | | | | A (SEQ ID NO: 100) | (SEQ ID NO: 220) | | |
| 56970 | ATXN7L3 | NM_001098833.1 | NC_000017.11 | 44197712 | sense | CATCGCTCAGGAGATATAC | AGGCCATCGCTCAGGAGATATACGCGGACC | CGG | 2 |
| | | | | | | G (SEQ ID NO: 101) | (SEQ ID NO: 221) | | |
| 56970 | ATXN7L3 | NM_001098833.1 | NC_000017.11 | 44197233 | sense | GCAGCCGAATCGCCAACCG | AACAGCAGCCGAATCGCCAACCGCCGGTGA | CGG | 3 |
| | | | | | | C (SEQ ID NO: 102) | (SEQ ID NO: 222) | | |
| 56970 | ATXN7L3 | NM_001098833.1 | NC_000017.11 | 44195424 | sense | GCTTCGCAGCCTGCTAACC | AGGAGCTTCGCAGCCTGCTAACCACGGTGA | CGG | 8 |
| | | | | | | A (SEQ ID NO: 103) | (SEQ ID NO: 223) | | |
| 9968 | MED12 | NM_005120.2 | NC_000023.11 | 71130165 | sense | ACATCGACTGCTGGACAAT | ATCCACATCGACTGCTGGACAATGAGGATG | AGG | 28 |
| | | | | | | G (SEQ ID NO: 104) | (SEQ ID NO: 224) | | |
| 9968 | MED12 | NM_005120.2 | NC_000023.11 | 71122231 | antisense | CAGTGAGTAGTGCCAAACC | CAGTCAGTGAGTAGTGCCAAACCAAGGCAC | AGG | 8 |
| | | | | | | A (SEQ ID NO: 105) | (SEQ ID NO: 225) | | |
| 9968 | MED12 | NM_005120.2 | NC_000023.11 | 71125111 | antisense | GTGGCGTACTGCACGTGTC | ATGGGTGGCGTACTGCACGTGTCGTGGCTG | TGG | 15 |
| | | | | | | G (SEQ ID NO: 106) | (SEQ ID NO: 226) | | |
| 9968 | MED12 | NM_005120.2 | NC_000023.11 | 71126138 | sense | TTCACATTATGACCAACAC | ACCTTTCACATTATGACCAACACCAGGTCA | AGG | 18 |
| | | | | | | C (SEQ ID NO: 107) | (SEQ ID NO: 227) | | |
| 3190 | HNRNPK | NM_002140.3 | NC_000009.12 | 83972098 | sense | ATGATGTTTGATGACCGTC | TACAATGATGTTTGATGACCGTCGCGGACG | CGG | 11 |
| | | | | | | G (SEQ ID NO: 108) | (SEQ ID NO: 228) | | |
| 3190 | HNRNPK | NM_002140.3 | NC_000009.12 | 83975465 | antisense | CTGTTGGGACATACCGCTC | TAAACTGTTGGGACATACCGCTCGGGGCCA | GGG | 6 |
| | | | | | | G (SEQ ID NO: 109) | (SEQ ID NO: 229) | | |
| 3190 | HNRNPK | NM_002140.3 | NC_000009.12 | 83971978 | sense | GATGATATGAGCCCTCGTC | TTATGATGATATGAGCCCTCGTCGAGGACC | AGG | 11 |
| | | | | | | G (SEQ ID NO: 110) | (SEQ ID NO: 230) | | |
| 3190 | HNRNPK | NM_002140.3 | NC_000009.12 | 83973291 | sense | TAAAATCAAAGAACTTCGA | GTGCTAAAATCAAAGAACTTCGAGAGGTAA | AGG | 9 |
| | | | | | | G (SEQ ID NO: 111) | (SEQ ID NO: 231) | | |
| 23528 | ZNF281 | NM_001281293.1 | NC_000001.11 | 200408377 | antisense | CCTCCACTGGAAGACACGG | TATGCCTCCACTGGAAGACACGGTAGGCAT( | AGG | 2 |
| | | | | | | T (SEQ ID NO: 112) | SEQ ID NO: 232) | | |
| 23528 | ZNF281 | NM_001281293.1 | NC_000001.11 | 200409263 | antisense | CGAACAGCCCCCCATAGTG | CCAGCGAACAGCCCCCCATAGTGGTGGTGG | TGG | 2 |
| | | | | | | G (SEQ ID NO: 113) | (SEQ ID NO: 233) | | |
| 23528 | ZNF281 | NM_001281293.1 | NC_000001.11 | 200409484 | antisense | GAGGATAACACGCATTGCG | AGAGGAGGATAACACGCATTGCGGGGGAGG | GGG | 2 |
| | | | | | | G (SEQ ID NO: 114) | (SEQ ID NO: 234) | | |
| 23528 | ZNF281 | NM_001281293.1 | NC_000001.11 | 200409128 | antisense | TGCTGAGTAATACGTCACG | CTGCTGCTGAGTAATACGTCACGGTGGTGC | TGG | 2 |
| | | | | | | G (SEQ ID NO: 115) | (SEQ ID NO: 235) | | |
| 27097 | TAF5L | NM_014409.3 | NC_000001.11 | 229602246 | antisense | CGGGACACGTCTACTTGGT | GATGCGGGACACGTCTACTTGGTGGGGCTC | GGG | 4 |
| | | | | | | G (SEQ ID NO: 116) | (SEQ ID NO: 236) | | |
| 27097 | TAF5L | NM_014409.3 | NC_000001.11 | 229602452 | sense | GCAGAACGAGGCTGCCCTA | TTCTGCAGAACGAGGCTGCCCTAGAGGTCT | AGG | 4 |
| | | | | | | G (SEQ ID NO: 117) | (SEQ ID NO: 237) | | |
| 27097 | TAF5L | NM_014409.3 | NC_000001.11 | 229595026 | sense | GCGGACCAGTGTACAGCAC | CACTGCGGACCAGTGTACAGCACGAGGTTC | AGG | 5 |
| | | | | | | G (SEQ ID NO: 118) | (SEQ ID NO: 238) | | |
| 27097 | TAF5L | NM_014409.3 | NC_000001.11 | 229602605 | antisense | TAAGGTGAGGACTTTGCAC | TATGTAAGGTGAGGACTTTGCACAGGGCAG | GGG | 4 |
| | | | | | | A (SEQ ID NO: 119) | (SEQ ID NO: 239) | | |
| 1649 | DDIT3 | NM_001195057.1 | NC_000012.12 | 57517292 | antisense | ATTTCCAGGAGGTGAAACA | CTTCATTTCCAGGAGGTGAAACATAGGTAC | AGG | 3 |
| | | | | | | T (SEQ ID NO: 120) | (SEQ ID NO: 240) | | |
| 1649 | DDIT3 | NM_001195057.1 | NC_000012.12 | 57517331 | sense | CTGGTATGAGGACCTGCAA | AAGCCTGGTATGAGGACCTGCAAGAGGTCC | AGG | 3 |
| | | | | | | G (SEQ ID NO: 121) | (SEQ ID NO: 241) | | |
| 1649 | DDIT3 | NM_001195057.1 | NC_000012.12 | 57517077 | antisense | GACTGGAATCTGGAGAGTG | CTCTGACTGGAATCTGGAGAGTGAGGGCTC | GGG | 4 |
| | | | | | | A (SEQ ID NO: 122) | (SEQ ID NO: 242) | | |
| 1649 | DDIT3 | NM_001195057.1 | NC_000012.12 | 57517146 | antisense | TCAGCCAAGCCAGAGAAG | TCAGTCAGCCAAGCCAGAGAAGCAGGGTCA | GGG | 4 |
| | | | | | | CA (SEQ ID NO: 123) | (SEQ ID NO: 243) | | |
| 23613 | ZMYND8 | NM_001281775.2 | NC_000020.11 | 47291845 | antisense | AGATGTATTCCGCATAGTC | TGGAAGATGTATTCCGCATAGTCAGGGTGC | GGG | 6 |
| | | | | | | A (SEQ ID NO: 124) | (SEQ ID NO: 244) | | |
| 23613 | ZMYND8 | NM_001281775.2 | NC_000020.11 | 47298798 | antisense | CACTTAGCGTGATAAACCC | CAGACACTTAGCGTGATAAACCCGGGGACA | GGG | 4 |
| | | | | | | G (SEQ ID NO: 125) | (SEQ ID NO: 245) | | |
| 23613 | ZMYND8 | NM_001281775.2 | NC_000020.11 | 47239052 | sense | CTCTTCCGCCCAAACTTCC | CCCGCTCTTCCGCCCAAACTTCCGCGGCTG | CGG | 15 |
| | | | | | | G (SEQ ID NO: 126) | (SEQ ID NO: 246) | | |
| 23613 | ZMYND8 | NM_001281775.2 | NC_000020.11 | 47276455 | antisense | GGAGCGCGGCATATCCGAC | TGGGGGAGCGCGGCATATCCGACAAGGAAA | AGG | 11 |
| | | | | | | A (SEQ ID NO: 127) | (SEQ ID NO: 247) | | |
| 9282 | MED14 | NM_004229.3 | NC_000023.11 | 40692233 | antisense | ATCACACATAGCGACGAA | TTGTATCACACATAGCGACGAAGTGGGCTA | GGG | 15 |
| | | | | | | GT (SEQ ID NO: 128) | (SEQ ID NO: 248) | | |
| 9282 | MED14 | NM_004229.3 | NC_000023.11 | 40714644 | antisense | CAGAGCATCTCTAGCTAAC | GGACCAGAGCATCTCTAGCTAACGAGGCCA | AGG | 4 |
| | | | | | | G (SEQ ID NO: 129) | (SEQ ID NO: 249) | | |
| 9282 | MED14 | NM_004229.3 | NC_000023.11 | 40682898 | antisense | CTAACTCTGCTACCCAAGT | AACACTAACTCTGCTACCCAAGTGCGGTTA | CGG | 17 |
| | | | | | | G (SEQ ID NO: 130) | (SEQ ID NO: 250) | | |
| 9282 | MED14 | NM_004229.3 | NC_000023.11 | 40711237 | sense | TAATGTTAATCCGAGAACG | ACTCTAATGTTAATCCGAGAACGGTGGGGA | TGG | 8 |
| | | | | | | G (SEQ ID NO: 131) | (SEQ ID NO: 251) | | |
| 5885 | RAD21 | NM_006265.2 | NC_000008.11 | 116856232 | antisense | AAGTGTTGTTTGATCAGTC | GAACAAGTGTTGTTTGATCAGTCATGGTTG | TGG | 8 |
| | | | | | | A (SEQ ID NO: 132) | (SEQ ID NO: 252) | | |
| 5885 | RAD21 | NM_006265.2 | NC_000008.11 | 116861852 | antisense | ACATACTCTAAGTCAGGCA | AGACACATACTCTAAGTCAGGCAGTGGCTG | TGG | 4 |
| | | | | | | G (SEQ ID NO: 133) | (SEQ ID NO: 253) | | |
| 5885 | RAD21 | NM_006265.2 | NC_000008.11 | 116866612 | sense | GTGTAATTTAGAGAGCAGC | TCGAGTGTAATTTAGAGAGCAGCGTGGAGA | TGG | 2 |
| | | | | | | G (SEQ ID NO: 134) | (SEQ ID NO: 254) | | |
| 5885 | RAD21 | NM_006265.2 | NC_000008.11 | 116857380 | antisense | TCTGTTCAGACTCTAATAG | GTGCTCTGTTCAGACTCTAATAGGAGGTTA | AGG | 6 |
| | | | | | | G (SEQ ID NO: 135) | (SEQ ID NO: 255) | | |
| 55929 | DMAP1 | NM_001034023.1 | NC_000001.11 | 44218708 | sense | ATGCTGGGCACGAACGAC | TTTGATGCTGGGCACGAACGACGGCGGAAG | CGG | 6 |
| | | | | | | GG (SEQ ID NO: 136) | (SEQ ID NO: 256) | | |
| 55929 | DMAP1 | NM_001034023.1 | NC_000001.11 | 44218427 | antisense | CATGGATAACAACAAAAC | CGGTCATGGATAACAACAAAACGCAGGTCA | AGG | 5 |
| | | | | | | GC (SEQ ID NO: 137) | (SEQ ID NO: 257) | | |
| 55929 | DMAP1 | NM_001034023.1 | NC_000001.11 | 44219225 | sense | GAAGCTACCCCAGAAAAA | AAAAGAAGCTACCCCAGAAAAAGGAGGCTG | AGG | 7 |
| | | | | | | GG (SEQ ID NO: 138) | (SEQ ID NO: 258) | | |
| 55929 | DMAP1 | NM_001034023.1 | NC_000001.11 | 44213854 | sense | GGACATTATCAACCCGGAC | AGAAGGACATTATCAACCCGGACAAGGTAG | AGG | 2 |
| | | | | | | A (SEQ ID NO: 139) | (SEQ ID NO: 259) | | |
| 80317 | ZKSCAN3 | NM_001242894.1 | NC_000006.12 | 28363758 | sense | CACAGCAGGATTCATCTCA | TGGACACAGCAGGATTCATCTCAGGGGAAT | GGG | 6 |
| | | | | | | G (SEQ ID NO: 140) | (SEQ ID NO: 260) | | |
| 80317 | ZKSCAN3 | NM_001242894.1 | NC_000006.12 | 28359769 | antisense | GCCGACTCAGCGCCTCGCG | CGGAGCCGACTCAGCGCCTCGCGGGGGCCT | GGG | 3 |
| | | | | | | G (SEQ ID NO: 141) | (SEQ ID NO: 261) | | |
| 80317 | ZKSCAN3 | NM_001242894.1 | NC_000006.12 | 28365662 | sense | GCTCAGGCCTGAGTAAACA | CAAAGCTCAGGCCTGAGTAAACACAGGAGA | AGG | 7 |
| | | | | | | C (SEQ ID NO: 142) | (SEQ ID NO: 262) | | |
| 80317 | ZKSCAN3 | NM_001242894.1 | NC_000006.21 | 28365539 | antisense | TCACCAGCTTCTGCACATG | CTGTTCACCAGCTTCTGCACATGTAGGAAT | AGG | 7 |
| | | | | | | T (SEQ ID NO: 143) | (SEQ ID NO: 263) | | |
| 27086 | FOXP1 | NM_032682.5 | NC_000003.12 | 71041428 | antisense | AGAGGAGGAGACACATGT | GTGCAGAGGAGGAGACACATGTCGTGGTCA | TGG | 11 |
| | | | | | | CG (SEQ ID NO: 144) | (SEQ ID NO: 264) | | |
| 27086 | FOXP1 | NM_032682.5 | NC_000003.21 | 71015617 | antisense | CATACACCATGTCCATAGA | CTTGCATACACCATGTCCATAGAGAGGATG | AGG | 12 |
| | | | | | | G (SEQ ID NO: 145) | (SEQ ID NO: 265) | | |
| 27086 | FOXP1 | NM_032682.5 | NC_000003.12 | 71046982 | sense | GCCTTCTGACAATTCAGCC | CAAGGCCTTCTGACAATTCAGCCCGGGCAG | GGG | 10 |
| | | | | | | C (SEQ ID NO: 146) | (SEQ ID NO: 266) | | |
| 27086 | FOXP1 | NM_032682.5 | NC_000003.12 | 70988031 | antisense | GTTCTGTAGACTTCACATG | TTGGGTTCTGTAGACTTCACATGCAGGTGG | AGG | 14 |
| | | | | | | C (SEQ ID NO: 147) | (SEQ ID NO: 267) | | |
| 6777 | STAT5B | NM_012448.3 | NC_000017.11 | 42216055 | sense | CAGCCAGGACAACAATGC | ATGGCAGCCAGGACAACAATGCGACGGCCA | CGG | 12 |
| | | | | | | GA (SEQ ID NO: 148) | (SEQ ID NO: 268) | | |
| 6777 | STAT5B | NM_012448.3 | NC_000017.11 | 42227658 | antisense | GTGGCCTTAATGTTCTCCT | CTGGGTGGCCTTAATGTTCTCCTGTGGATT | TGG | 3 |
| | | | | | | G (SEQ ID NO: 149) | (SEQ ID NO: 269) | | |
| 6777 | STAT5B | NM_012448.3 | NC_000017.11 | 42224822 | antisense | GTTCATTGTACAATATATG | CTCTGTTCATTGTACAATATATGGCGGATG | CGG | 4 |
| | | | | | | G (SEQ ID NO: 150) | (SEQ ID NO: 270) | | |
| 6777 | STAT5B | NM_012448.3 | NC_000017.11 | 42217252 | sense | TAAGAGGTCAGACCGTCGT | GAATTAAGAGGTCAGACCGTCGTGGGGCAG | GGG | 11 |
| | | | | | | G (SEQ ID NO: 151) | (SEQ ID NO: 271) | | |

As described herein, the stability of Treg cells may be modified by inhibiting the expression of the one or more nuclear factors set forth in Table 1 or Table 2. The stability of Treg cells may also be modified by overexpressing one or more nuclear factors set forth in Table 1 or Table 2. Subsequently, once modified Treg cells are created, the modified Treg cells may be administered to a human. Depending on whether the Treg cells are stabilized or destabilized, the modified Treg cells may be used to treat different indications. For example, Treg cells may be isolated from a whole blood sample of a human and expanded ex vivo. The expanded Treg cells may then be treated to inhibit the expression of a nuclear factor set forth in Table 1 or Table 2 thus, creating modified Treg cells. The modified Treg cells may be reintroduced to the human to treat certain indications. In some embodiments, destabilized Treg cells having less immunosuppressive effects may be used to treat cancer. In some embodiments, stabilized Treg cells having improved immunosuppressive effects may be used to treat autoimmune diseases. Certain nuclear factors in Treg cells increase Foxp3 expression (Table 1) and have a stabilizing effect once their expression is inhibited, while other nuclear factors decrease Foxp3 expression (Table 2) in Treg cells and have a destabilizing effect once their expression is inhibited. Cell stability may be determined by a multi-color FACS panel based on Treg cell markers like Foxp3, Helios, CTLA-4, CD25, IL-10, and effectors such as cytokines typically associated with effector T cell subsets like IL-2, IFNγ, IL-17a, and IL-4. Assays for measuring Treg cell stability can be found in, e.g., McClymont, et al., “Plasticity of Human Regulatory T Cells in Healthy Subjects and Patients with Type 1 Diabetes” J. immunol. 186 (2011). Depending on the indication and therapeutic needs, one may choose to target one or more nuclear factors to generate modified Treg cells that are destabilized or stabilized.

In other cases, Treg cells in a subject can be modified in vivo, for example, by using a targeted vector, such as, a lentiviral vector, a retroviral vector an adenoviral or adeno-associated viral vector. In vivo delivery of targeted nucleases that modify the genome of a Treg cell can also be used. See for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017).

Also provided is a Treg cell wherein expression of one or more nuclear factors set forth in Table 1 or Table 2 is inhibited. Further provided is a Treg cell wherein one or more nuclear factors set forth in Table 1 or Table 2 is overexpressed. The disclosure also features a Treg cell comprising a genetic modification or heterologous polynucleotide that inhibits expression of one or more nuclear factors set forth in Table 1 and/or a heterologous polynucleotide that encodes a nuclear factor set forth in Table 2. Also provided is a Treg cell comprising a genetic modification or heterologous polynucleotide that inhibits expression of a nuclear factor set forth in Table 2 and/or a heterologous polynucleotide that encodes a nuclear factor set forth in Table 1.

A genetic modification may be a nucleotide mutation or any sequence alteration in the polynucleotide encoding the nuclear factor that results in the inhibition of the expression of the nuclear factor. A heterologous polynucleotide may refer to a polynucleotide originally encoding the nuclear factor but is altered, i.e., comprising one or more nucleotide mutations or sequence alterations. In some embodiments, the heterologous polynucleotide is inserted into the genome of the Treg cell by introducing a vector, for example, a viral vector, comprising the polynucleotide. Examples of viral vectors include, but are not limited to adeno-associated viral (AAV) vectors, retroviral vectors or lentiviral vectors. In some embodiments, the lentiviral vector is an integrase-deficient lentiviral vector.

Also disclosed herein are Treg cells comprising at least one guide RNA (gRNA) comprising a sequence selected from Table 3. The expression of one or more nuclear factors set forth in Table 1 or Table 2, in the Treg cells comprising the gRNAs, may be reduced in the Treg cells relative to the expression of the one or more nuclear factors in Treg cells not comprising the gRNAs. In other examples, an endogenous nuclear factor set forth in Table 1 or Table 2 can be inhibited by targeting a deactivated targeted nuclease, for example dCAs9, fused to a transcriptional repressor, to the promoter region of the endogenous nuclear factor gene. In other examples, an endogenous nuclear factor set forth in Table 1 or Table 2 can be upregulated or overexpressed by targeting a deactivated targeted nuclease, for example dCAs9, fused to a transcriptional activator, to the promoter region of the endogenous nuclear factor gene. See, for example, Qi et al. “The New State of the Art: Cas9 for Gene Activation and Repression,” Mol. and Cell. Biol., 35(22): 3800-3809 (2015).

### III. Methods of Inhibiting Expression

CRISPR/Cas Genome Editing

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease can require both the crRNA and the tracrRNA for site-specific DNA recognition and cleavage. This system has now been engineered such that the crRNA and tracrRNA can be combined into one molecule (the “guide RNA” or “gRNA”), and the crRNA equivalent portion of the single guide RNA can be engineered to guide the Cas (e.g., Cas9) nuclease to target any desired sequence (see, e.g., Jinek et al. (2012) Science 337:816-821; Jinek et al. (2013) eLife 2:e00471; Segal (2013) eLife 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments of the methods described herein, CRISPR/Cas genome editing may be used to inhibit the expression of one or more nuclear factors set forth in Table 1 or Table 2.

In some embodiments, the Cas nuclease has DNA cleavage activity. The Cas nuclease can direct cleavage of one or both strands at a location in a target DNA sequence, i.e., a location in a polynucleotide encoding a nuclear factor set forth in Table 1 or Table 2. In some embodiments, the Cas nuclease can be a nickase having one or more inactivated catalytic domains that cleaves a single strand of a target DNA sequence.

Non-limiting examples of Cas nucleases include Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. Some CRISPR-related endonucleases that may be used in methods described herein are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 may be a fusion protein, e.g., the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the Cas9 nuclease may be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break may be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of INDEL mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10M and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme may contain a mutation at D10, E762, H983, or D986, as well as a mutation at H840 or N863. In some instances, the dCas9 enzyme may contain a D10A or DION mutation. Also, the dCas9 enzyme may contain a H840A, H840Y, or H840N. In some embodiments, the dCas9 enzyme may contain D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

As described above, a gRNA may comprise a crRNA and a tracrRNAs. The gRNA can be configured to form a stable and active complex with a gRNA-mediated nuclease (e.g., Cas9 or dCas9). The gRNA contains a binding region that provides specific binding to the target genetic element. Exemplary gRNAs that may be used to target a region in a polynucleotide encoding a nuclear factor set forth in Table 1 or Table 2 are listed in Table 3 below. A gRNA used to target a region in a polynucleotide encoding a nuclear factor set forth in Table 1 or Table 2 may comprise a sequence selected from Table 3 below or a portion thereof.

In some embodiments, the targeted nuclease, for example, a Cpf1 nuclease or a Cas9 nuclease and the gRNA are introduced into the Treg cell as a ribonucleoprotein (RNP) complex. In some embodiments, the RNP complex may be introduced into about 1×10⁵ to about 2×10⁶ cells (e.g., 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells, or about 1×10⁶ cells to about 2×10⁶ cells). In some embodiments, the Treg cells are cultured under conditions effective for expanding the population of modified Treg cells. Also disclosed herein is a population of Treg cells, in which the genome of at least 20%, 30%, 40%, 50%, 60%, 70%, 80%, 90%, 95%, 99% or greater of the cells comprises a genetic modification or heterologous polynucleotide that inhibits expression of one or more nuclear factors set forth in Table 1 or Table 2.

In some embodiments, the RNP complex is introduced into the Treg cells by electroporation. Methods, compositions, and devices for electroporating cells to introduce a RNP complex are available in the art, see, e.g., WO 2016/123578, WO/2006/001614, and Kim, J. A. et al. Biosens. Bioelectron. 23, 1353-1360 (2008). Additional or alternative methods, compositions, and devices for electroporating cells to introduce a RNP complex can include those described in U.S. Patent Appl. Pub. Nos. 2006/0094095; 2005/0064596; or 2006/0087522; Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842; Geng, T. et al., J. Control Release 144, 91-100 (2010); and Wang, J., et al. Lab. Chip 10, 2057-2061 (2010).

In some embodiments, the sequence of the gRNA or a portion thereof is designed to complement (e.g., perfectly complement) or substantially complement (e.g., 50%, 55%, 60%, 65%, 70%, 75%, 80%, 85%, 90%, 91%, 92%, 93%, 94% 95%, 96%, 97%, 98%, or 99% complement) the target region in the polynucleotide encoding the protein. In some embodiments, the portion of the gRNA that complements and binds the targeting region in the polynucleotide is, or is about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more nucleotides in length. In some cases, the portion of the gRNA that complements and binds the targeting region in the polynucleotide is between about 19 and about 21 nucleotides in length. In some cases, the gRNA may incorporate wobble or degenerate bases to bind target regions. In some cases, the gRNA can be altered to increase stability. For example, non-natural nucleotides, can be incorporated to increase RNA resistance to degradation. In some cases, the gRNA can be altered or designed to avoid or reduce secondary structure formation. In some cases, the gRNA can be designed to optimize G-C content. In some cases, G-C content is between about 40% and about 60% (e.g., 40%, 45%, 50%, 55%, 60%). In some cases, the binding region can contain modified nucleotides such as, without limitation, methylated or phosphorylated nucleotides

In some embodiments, the gRNA can be optimized for expression by substituting, deleting, or adding one or more nucleotides. In some cases, a nucleotide sequence that provides inefficient transcription from an encoding template nucleic acid can be deleted or substituted. For example, in some cases, the gRNA is transcribed from a nucleic acid operably linked to an RNA polymerase III promoter. In such cases, gRNA sequences that result in inefficient transcription by RNA polymerase III, such as those described in Nielsen et al., Science. 2013 Jun. 28; 340(6140):1577-80, can be deleted or substituted. For example, one or more consecutive uracils can be deleted or substituted from the gRNA sequence. In some cases, if the uracil is hydrogen bonded to a corresponding adenine, the gRNA sequence can be altered to exchange the adenine and uracil. This “A-U flip” can retain the overall structure and function of the gRNA molecule while improving expression by reducing the number of consecutive uracil nucleotides.

In some embodiments, the gRNA can be optimized for stability. Stability can be enhanced by optimizing the stability of the gRNA:nuclease interaction, optimizing assembly of the gRNA:nuclease complex, removing or altering RNA destabilizing sequence elements, or adding RNA stabilizing sequence elements. In some embodiments, the gRNA contains a 5′ stem-loop structure proximal to, or adjacent to, the region that interacts with the gRNA-mediated nuclease. Optimization of the 5′ stem-loop structure can provide enhanced stability or assembly of the gRNA:nuclease complex. In some cases, the 5′ stem-loop structure is optimized by increasing the length of the stem portion of the stem-loop structure.

gRNAs can be modified by methods known in the art. In some cases, the modifications can include, but are not limited to, the addition of one or more of the following sequence elements: a 5′ cap (e.g., a 7-methylguanylate cap); a 3′ polyadenylated tail; a riboswitch sequence; a stability control sequence; a hairpin; a subcellular localization sequence; a detection sequence or label; or a binding site for one or more proteins. Modifications can also include the introduction of non-natural nucleotides including, but not limited to, one or more of the following: fluorescent nucleotides and methylated nucleotides.

Also described herein are expression cassettes and vectors for producing gRNAs in a host cell. The expression cassettes can contain a promoter (e.g., a heterologous promoter) operably linked to a polynucleotide encoding a gRNA. The promoter can be inducible or constitutive. The promoter can be tissue specific. In some cases, the promoter is a U6, H1, or spleen focus-forming virus (SFFV) long terminal repeat promoter. In some cases, the promoter is a weak mammalian promoter as compared to the human elongation factor 1 promoter (EF1A). In some cases, the weak mammalian promoter is a ubiquitin C promoter or a phosphoglycerate kinase 1 promoter (PGK). In some cases, the weak mammalian promoter is a TetOn promoter in the absence of an inducer. In some cases, when a TetOn promoter is utilized, the host cell is also contacted with a tetracycline transactivator. In some embodiments, the strength of the selected gRNA promoter is selected to express an amount of gRNA that is proportional to the amount of Cas9 or dCas9. The expression cassette can be in a vector, such as a plasmid, a viral vector, a lentiviral vector, etc. In some cases, the expression cassette is in a host cell. The gRNA expression cassette can be episomal or integrated in the host cell.

Zinc-Finger Nucleases (ZFNs)

“Zinc finger nucleases” or “ZFNs” are a fusion between the cleavage domain of FokI and a DNA recognition domain containing 3 or more zinc finger motifs. The heterodimerization at a particular position in the DNA of two individual ZFNs in precise orientation and spacing leads to a double-strand break in the DNA. In some embodiments of the methods described herein, ZFNs may be used to inhibit the expression of one or more nuclear factors set forth in Table 1 or Table 2, i.e., by cleaving the polynucleotide encoding the protein.

In some cases, ZFNs fuse a cleavage domain to the C-terminus of each zinc finger domain. In order to allow the two cleavage domains to dimerize and cleave DNA, the two individual ZFNs bind opposite strands of DNA with their C-termini at a certain distance apart. In some cases, linker sequences between the zinc finger domain and the cleavage domain requires the 5′ edge of each binding site to be separated by about 5-7 bp. Exemplary ZFNs that may be used in methods described herein include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140.

ZFNs can generate a double-strand break in a target DNA, resulting in DNA break repair which allows for the introduction of gene modification. DNA break repair can occur via non-homologous end joining (NHEJ) or homology-directed repair (HDR). In HDR, a donor DNA repair template that contains homology arms flanking sites of the target DNA can be provided.

In some embodiments, a ZFN is a zinc finger nickase which can be an engineered ZFN that induces site-specific single-strand DNA breaks or nicks, thus resulting in HDR. Descriptions of zinc finger nickases are found, e.g., in Ramirez et al., Nucl Acids Res, 2012, 40(12):5560-8; Kim et al., Genome Res, 2012, 22(7):1327-33.

TALENs

TALENS may also be used to inhibit the expression of one or more nuclear factors set forth in Table 1 or Table 2. “TALENs” or “TAL-effector nucleases” are engineered transcription activator-like effector nucleases that contain a central domain of DNA-binding tandem repeats, a nuclear localization signal, and a C-terminal transcriptional activation domain. In some instances, a DNA-binding tandem repeat comprises 33-35 amino acids in length and contains two hypervariable amino acid residues at positions 12 and 13 that can recognize one or more specific DNA base pairs. TALENs can be produced by fusing a TAL effector DNA binding domain to a DNA cleavage domain. For instance, a TALE protein may be fused to a nuclease such as a wild-type or mutated FokI endonuclease or the catalytic domain of FokI. Several mutations to Fold have been made for its use in TALENs, which, for example, improve cleavage specificity or activity. Such TALENs can be engineered to bind any desired DNA sequence.

TALENs can be used to generate gene modifications by creating a double-strand break in a target DNA sequence, which in turn, undergoes NHEJ or HDR. In some cases, a single-stranded donor DNA repair template is provided to promote HDR.

Detailed descriptions of TALENs and their uses for gene editing are found, e.g., in U.S. Pat. Nos. 8,440,431; 8,440,432; 8,450,471; 8,586,363; and U.S. Pat. No. 8,697,853; Scharenberg et al., Curr Gene Ther, 2013, 13(4):291-303; Gaj et al., Nat Methods, 2012, 9(8):805-7; Beurdeley et al., Nat Commun, 2013, 4:1762; and Joung and Sander, Nat Rev Mol Cell Biol, 2013, 14(1):49.

Meganucleases

Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some embodiments of the methods described herein, meganucleases may be used to inhibit the expression of one or more nuclear factors set forth in Table 1 or Table 2 i.e., by cleaving in a target region within the polynucleotide encoding the nuclear factor. In some instances, the meganuclease is naturally-occurring (found in nature) or wild-type, and in other instances, the meganuclease is non-natural, artificial, engineered, synthetic, or rationally designed. In certain embodiments, the meganucleases that may be used in methods described herein include, but are not limited to, an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Natl Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

RNA-Based Technologies

Various RNA-based technologies may also be used in methods described herein to inhibit the expression of one or more nuclear factors set forth in Table 1 or Table 2. Examples of RNA-based technologies include, but are not limited to, small interfering RNA (siRNA), antisense RNA, microRNA (miRNA), and short hairpin RNA (shRNA).

RNA-based technologies may use an siRNA, an antisense RNA, a miRNA, or a shRNA to target a sequence, or a portion thereof, that encodes a transcription factor. In some embodiments, one or more genes regulated by a transcription factor may also be targeted by an siRNA, an antisense RNA, a miRNA, or a shRNA. An siRNA, an antisense RNA, a miRNA, or a shRNA may target a sequence comprising at least 10, at least 20, at least 30, at least 40, at least 50, at least 60, at least 70, at least 80, at least 90, or at least 100 contiguous nucleotides.

An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by a protein called Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

The shRNA or siRNA may be encoded in a vector. In some embodiments, the vector further comprises appropriate expression control elements known in the art, including, e.g., promoters (e.g., inducible promoters or tissue specific promoters), enhancers, and transcription terminators.

### IV. Methods of Treatment

Any of the methods described herein may be used to modify Treg cells in a human subject or obtained from a human subject. Any of the methods and compositions described herein may be used to modify Treg cells obtained from a human subject to treat or prevent a disease (e.g., cancer, an autoimmune disease, an infectious disease, transplantation rejection, graft vs. host disease or other inflammatory disorder in a subject).

Provided herein is a method of treating an autoimmune disorder in a subject, the method comprising administering a population of Treg cells comprising a genetic modification or heterologous polynucleotide that inhibits expression of a nuclear factor set forth in Table 1 and/or a heterologous polynucleotide that encodes a nuclear factor set forth in Table 2 to a subject that has an autoimmune disorder.

Also provided is a method of treating cancer in a subject, the method comprising administering a population of Treg cells comprising a genetic modification or heterologous polynucleotide that inhibits expression of a nuclear factor set forth in Table 2 and/or a heterologous polynucleotide that encodes a nuclear factor set forth in Table 1 to a subject that has cancer.

Provided herein is a method of treating cancer in a human subject comprising: a) obtaining Treg cells from the subject; b) modifying the Treg cells using any of the methods provided herein to decrease the stability of the Treg cells; and c) administering the modified Treg cells to the subject, wherein the human subject has cancer. Also provided herein is a method of treating an autoimmune disease in a human subject comprising: a) obtaining Treg cells from the subject; b) modifying the Treg cells using any of the methods provided herein to increase the stability of the Treg cells; and c) administering the modified Treg cells to the subject, wherein the human subject has an autoimmune disease.

In some embodiments, Treg cells obtained from a cancer subject may be expanded ex vivo. The characteristics of the subject's cancer may determine a set of tailored cellular modifications (i.e., which nuclear factors from Table 1 and/or Table 2 to target), and these modifications may be applied to the Treg cells using any of the methods described herein. Modified Treg cells may then be reintroduced to the subject. This strategy capitalizes on and enhances the function of the subject's natural repertoire of cancer specific T cells, providing a diverse arsenal to eliminate mutagenic cancer cells quickly. Similar strategies may be applicable for the treatment of autoimmune diseases, in which the modified Treg cells would have improved stability.

In other cases, Treg cells in a subject can be targeted for in vivo modification. See, for example, See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

The following examples are provided by way of illustration only and not by way of limitation. Those of skill in the art will readily recognize a variety of non-critical parameters that could be changed or modified to yield essentially the same or similar results.

### Mice

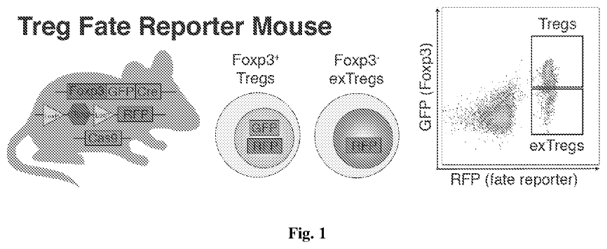

B6 Foxp3-GFP-Cre mice (Zhou et al., “Selective miRNA disruption in T reg cells leads to uncontrolled autoimmunity,” J Exp Med. 205, 1983-91 (2008)) were crossed with B6 Rosa26-RFP reporter mice (Luche et al., “Faithful activation of an extra-bright red fluorescent protein in “knock-in” Cre-reporter mice ideally suited for lineage tracing studies,” Eur. J. Immunol. 37, 43-53 (2007)) as previously described (Bailey-Bucktrout et al., “Self-antigen-driven activation induces instability of regulatory T cells during an inflammatory autoimmune response, Immunity. 39, 949-62 (2013)) to generate the Foxp3 fate reporter mice (FIG. 1). These mice were then crossed to B6 constitutive Cas9-expressing mice (Platt et al., “CRISPR-Cas9 knockin mice for genome editing and cancer modeling,” Cell. 159, 440-455 (2014)) to generate the Foxp3-GFP-Cre/Rosa26-RFP/Cas9 mice used for the CRISPR screen. For the arrayed validation experiments, B6 Foxp3-EGFP knockin mice that were obtained from Jackson Laboratories (Strain No. 006772) were used. All mice were maintained in the UCSF specific-pathogen-free animal facility in accordance with guidelines established by the Institutional Animal Care and Use Committee and Laboratory Animal Resource Center.

### Isolation and Culture of Primary Mouse Tregs

Spleens and peripheral lymph nodes were harvested from mice and dissociated in 1×PBS with 2% FBS and 1 mM EDTA. The mixture was then passed through a 70-μm filter. CD4+ T cells were isolated using the CD4+ Negative Selection Kit (StemCell Technologies, Cat #19752) followed by fluorescence-activated cell sorting. For the prescreen sort, Tregs were gated on lymphocytes, live cells, CD4+, CD62L+, RFP+, Foxp3-GFP+ cells. For the arrayed validation experiments, Tregs were gated on lymphocytes, live cells, CD4+, Foxp3-GFP+ cells. Sorted Tregs were cultured in complete DMEM, 10% FBS, 1% pen/strep+2000U hIL-2 in 24 well plates at 1 million cells/mL. Tregs were stimulated using CD3/CD28 Mouse T-Activator Dynabeads (Thermo Fisher, Cat #11456D) at a ratio of 3:1 beads to cells for 48 hours. Cells were split and media was refreshed every 2-3 days.

Pooled sgRNA Library Design and Construction

For the cloning of the targeted library, we followed the custom sgRNA library cloning protocol as previously described (Joung et al., “Genome-scale CRISPR-Cas9 knockout and transcriptional activation screening,” Nat Protocols. 12, 828-863 (2017)). We utilized a MSCV-U6-sgRNA-IRES-Thy1.1 backbone. To optimize this plasmid for cloning the library, we first replaced the sgRNA with a 1.9 kb stuffer derived from the lentiGuide-Puro plasmid (Addgene, plasmid #52963) with flanking BsgI cut sites. This stuffer was excised using the BsgI restriction enzyme (NEB, Cat #R0559) and the linear backbone was gel purified (Qiagen, Cat #28706). We designed a targeted library to include all genes matching Gene Ontology for “Nucleic Acid Binding Transcription Factors”, “Protein Binding Transcription Factors”, “Involved in Chromatin Organization” and “Involved in Epigenetic Regulation”. Genes were then selected based on those that have the highest expression levels across any mouse CD4 T cell subset as defined by Stubbington et al. (Stubbington et al., “An atlas of mouse CD4+ T cell transcriptomes,” Biol Direct. 10. 14 (2015)). In total, we included 493 targets with 4 guides per gene, and 28 non-targeting controls. Guides were subsetted from the Brie sgRNA library (Doench et al., “Optimized sgRNA design to maximize activity and minimize off-target effects of CRISPR-Cas9,” Nature biotechnology. 34 (2), 184-191 (2016)), and the pooled oligo library was ordered from Twist Bioscience to match the vector backbone. Oligos were PCR amplified and cloned into the modified MSCV backbone by Gibson assembly as described by Joung et al. The library was amplified using Endura ElectroCompetent Cells following the manufacturer's protocol (Endura, Cat #60242-1).

### Retrovirus Production

Platinum-E (Plat-E) Retroviral Packaging cells (Cell Biolabs, Inc., Cat #RV-101) were seeded at 10 million cells in 15 cm poly-L-Lysine coated dishes 16 hours prior to transfection and cultured in complete DMEM, 10% FBS, 1% pen/strep, 1 μg/mL puromycin and 10 μg/mL blasticidin Immediately before transfection, the media was replaced with antibiotic free complete DMEM, 10% FBS. The cells were transfected with the sgRNA transfer plasmids (MSCV-U6-sgRNA-IRES-Thy1.1) using TransIT-293 transfection reagent per the manufacturer's protocol (Mirus, Cat #MIR 2700). The following morning, the media was replaced with complete DMEM, 10% FBS, 1% pen/strep. The viral supernatant was collected 48 hours post-transfection and filtered through a 0.45 μm, polyethersulfone sterile syringe filter (Whatman, Cat #6780-2504), to remove cell debris. The viral supernatant was aliquoted and stored until use at −80° C.

### Retroviral Transduction

Tregs were stimulated as described above for 48-60 hours. Cells were counted and seeded at 3 million cells in 1 mL of media with 2×hIL-2 into each well of a 6 well plate that was coated with 15 μg/mL of RetroNectin (Takara, Cat #T100A) for 3 hours at room temperature and subsequently washed with 1×PBS. Retrovirus was added at a 1:1 v/v ratio (1 mL) and plates were centrifuged for 1 hour at 2000 g at 30° C. and placed in the incubator at 37° C. overnight. The next day, half (1 mL) of the 1:1 retrovirus to media mixture was removed from the plate and 1 mL of fresh retrovirus was added. Plates were immediately centrifuged for 1 hour at 2000 g at 30° C. After the second spinfection, cells were pelleted, washed, and cultured in fresh media.

### Foxp3 Intracellular Stain and Post-Screen Cell Collection

Tregs were collected from their culture vessels 8 days after the second transduction and centrifuged for 5 min at 300 g. Cells were first stained with a viability dye at a 1:1,000 dilution in 1×PBS for 20 min at 4° C., then washed with EasySep Buffer (1×PBS, 2% FBS, 1 mM EDTA). Cells were then resuspended in the appropriate surface staining antibody cocktail and incubated for 30 min at 4° C., then washed with EasySep Buffer. Cells were then fixed, permeabilized, and stained for transcription factors using the Foxp3 Transcription Factor Staining Buffer Set (eBioscience, Cat #00-5523-00) according to the manufacturer's instructions. For the CRISPR screen, Foxp3 high and Foxp3 low populations were isolated using fluorescence-activated cell sorting by gating on lymphocytes, live cells, CD4+ and gating on the highest 40% of Foxp3-expressing cells (Foxp3 high) and lowest 40% of Foxp3-expressing cells (Foxp3 low) by endogenous Foxp3 intracellular staining. Over 2 million cells were collected for both sorted populations to maintain a library coverage of at least 1,000 cells per sgRNA.

Isolation of Genomic DNA from Fixed Cells

After cell sorting and collection, genomic DNA (gDNA) was isolated using a protocol specific for fixed cells. Cell pellets were resuspended in cell lysis buffer (0.5% SDS, 50 mM Tris, pH 8, 10 mM EDTA) with 1:25 v/v of 5M NaCl to reverse crosslinking and incubated at 66° C. overnight. RNase A (10 mg/mL) was added at 1:50 v/v and incubated at 37° C. for 1 hour. Proteinase K (20 mg/mL) was added at 1:50 v/v and incubated at 45° C. for 1 hour. Phenol:Chloroform:Isoamyl Alcohol (25:24:1) was added to the sample 1:1 v/v and transferred to a phase lock gel light tube (QuantaBio, Cat #2302820), inverted vigorously and centrifuged at 20,000 g for 5 mins. The aqueous phase was then transferred to a clean tube and NaAc at 1:10 v/v, 1 μl of GeneElute-LPA (Sigma, Cat #56575), and isopropanol at 2.5:1 v/v were added. The sample was vortexed, and incubated at −80° C. until frozen solid. Then thawed and centrifuged at 20,000 g for 30 mins. The cell pellet was washed with 500 μl of 75% EtOH, gently inverted and centrifuged at 20,000 g for 5 mins, aspirated, dried, and resuspended in 20 μl TE buffer.

### Preparation of Genomic DNA for Next Generation Sequencing

Amplification and bar-coding of sgRNAs for the cell surface sublibrary was performed as previously described (Gilbert et al., “Genome scale CRISPR-mediated control of gene repression and activation,’ Cell. 159, 647-661 (2014)) with some modifications. Briefly, after gDNA isolation, sgRNAs were amplified and barcoded with TruSeq Single Indexes using a one-step PCR. TruSeq Adaptor Index 12 (CTTGTA) was used for the Foxp3 low population and TrueSeq Adaptor Index 14 (AGTTCC) was used for the Foxp3 high population. Each PCR reaction consisted of 50 μL of NEBNext Ultra II Q5 Master Mix (NEB #M0544), 1 μg of gDNA, 2.5 μL each of the 10 μM forward and reverse primers, and water to 1004, total. The PCR cycling conditions were: 3 minutes at 98° C., followed by 10 seconds at 98° C., 10 seconds at 62° C., 25 seconds at 72° C., for 26 cycles; and a final 2 minute extension at 72° C. After the PCR, the samples were purified using Agencourt AMPure XPSPRI beads (Beckman Coulter, cat #A63880) per the manufacturer's protocol, quantified using the Qubit ssDNA high sensitivity assay kit (Thermo Fisher Scientific, cat #Q32854), and then analyzed on the 2100 Bioanalyzer Instrument. Samples were then sequenced on an Illumina MiniSeq using a custom sequencing primer.

### Pooled CRISPR Screen Pipeline

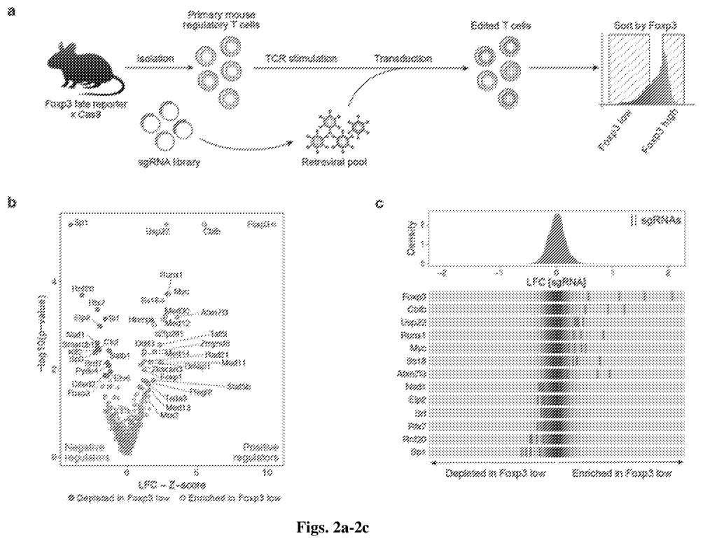

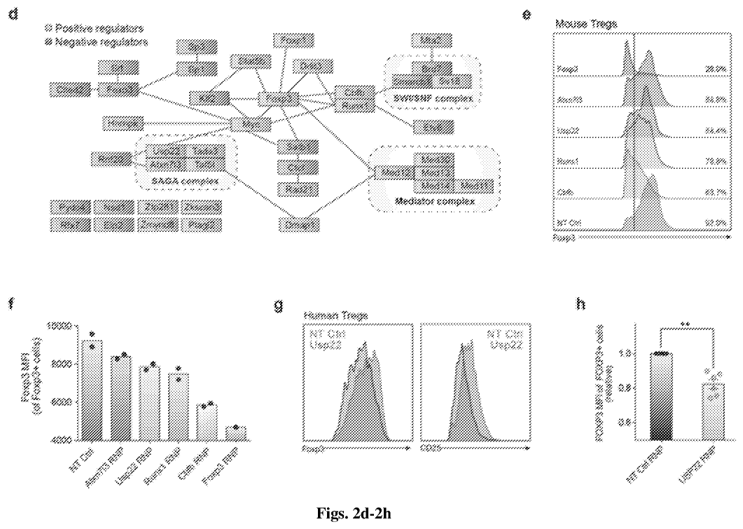

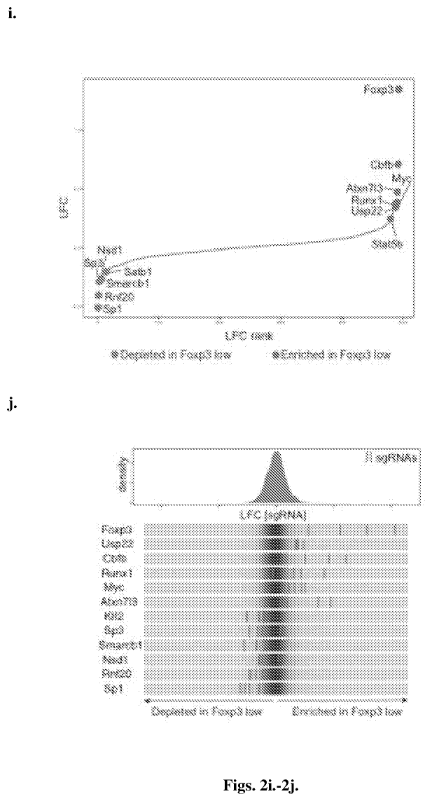

Primary Tregs were isolated from the spleen and lymph nodes of three male Foxp3-GFP-Cre/Rosa26-RFP/Cas9 mice aged 5-7 months old, pooled together, and stimulated for 60 hours. Cells were then retrovirally transduced with the sgRNA library and cultured at a density of 1 million cells/ml continually maintaining a library coverage of at least 1,000 cells per sgRNA. Eight days after the second transduction, cells were sorted based on Foxp3 expression defined by intracellular staining. Genomic DNA was harvested from each population and the sgRNA-encoding regions were then amplified by PCR and sequenced on an Illumina MiniSeq using custom sequencing primers. From this data, we quantified the frequencies of cells expressing different sgRNAs in each in each population (Foxp3 high and Foxp3 low) and quantified the phenotype of the sgRNAs, which we have defined as Foxp3 stabilizing (enriched in Foxp3 high) or Foxp3 destabilizing (enriched in Foxp3 low) (FIG. 2).

### Analysis of Pooled CRISPR Screen

Analysis was performed as previously described (Shifrut et al., “Genome-wide CRISPR Screens in Primary Human T Cells Reveal Key Regulators of Immune Function. Biorxiv. (2018)doi: https://doi.org/10.1101/384776)). To identify hits from the screen, we used the MAGeCK software to quantify and test for guide enrichment (Li et al., “MAGeCK enables robust identification of essential genes from genome-scale CRISPR/Cas9 knockout screens,” Genome Biol. 15, 554 (2014)). Abundance of guides was first determined by using the MAGeCK “count” module for the raw fastq files. For the targeted libraries the constant 5′ trim was automatically detected by MAGeCK. To test for robust guide and gene-level enrichment, the MAGeCK “test” module was used with default parameters. This step includes median ratio normalization to account for varying read depths. We used the non-targeting control guides to estimate the size factor for normalization, as well as to build the mean-variance model for null distribution, which is used to find significant guide enrichment. MAGeCK produced guide-level enrichment scores for each direction (i.e. positive and negative) which were then used for alpha-robust rank aggregation (RRA) to obtain gene-level scores. The p-value for each gene is determined by a permutation test, randomizing guide assignments and adjusted for false discovery rates by the Benjamini-Hochberg method. Log 2 fold change (LFC) is also calculated for each gene, defined throughout as the median LFC for all guides per gene target. Where indicated, LFC was normalized to have a mean of 0 and standard deviation of 1 to obtain the LFC Z-score.

### Arrayed Cas9 Ribonucleotide Protein (RNP) Preparation and Electroporation

RNPs were produced by complexing a two-component gRNA to Cas9, as previously described (Schumann et al., “Generation of knock-in primary human T cells using Cas9 ribonucleoproteins,” Proc. Natl Acad. Sci. USA. 112, 10437-10442 (2015)). In brief, crRNAs and tracrRNAs were chemically synthesized (IDT), and recombinant Cas9-NLS were produced and purified (QB3 Macrolab). Lyophilized RNA was resuspended in Nuclease-free Duplex Buffer (IDT, Cat #1072570) at a concentration of 160 μM, and stored in aliquots at −80° C. crRNA and tracrRNA aliquots were thawed, mixed 1:1 by volume, and annealed by incubation at 37° C. for 30 min to form an 80 μM gRNA solution. Recombinant Cas9 was stored at 40 μM in 20 mM HEPES-KOH, pH 7.5, 150 mM KCl, 10% glycerol, 1 mM DTT, were then mixed 1:1 by volume with the 80 μM gRNA (2:1 gRNA to Cas9 molar ratio) at 37° C. for 15 min to form an RNP at 20 μM. RNPs were electroporated immediately after complexing. RNPs were electroporated 3 days after initial stimulation, Tregs were collected from their culture vessels and centrifuged for 5 min at 300 g, aspirated, and resuspended in the Lonza electroporation buffer P3 using 20 μl buffer per 200,000 cells. 200,000 Tregs were electroporated per well using a Lonza 4D 96-well electroporation system with pulse code EO148. Immediately after electroporation, 80 μL of pre-warmed media was added to each well and the cells were incubated at 37° C. for 15 minutes. The cells were then transferred to a round-bottom 96-well tissue culture plate and cultured in complete DMEM, 10% FBS, 1% pen/strep+2000U hIL-2 at 200,000 cells/well in 200 μl of media.

### Isolation and Culture of Human Treg Cells

Primary human Treg cells for all experiments were obtained from residuals from leukoreduction chambers after Trima Apheresis (Blood Centers of the Pacific) under a protocol approved by the UCSF Committee on Human Research (CHR #13-11950). Peripheral blood mononuclear cells (PBMCs) were isolated from samples by Lymphoprep centrifugation (StemCell, Cat #07861) using SepMate tubes (StemCell, Cat #85460). CD4+ T cells were isolated from PBMCs by magnetic negative selection using the EasySep Human CD4+ T Cell Isolation Kit (StemCell, Cat #17952) and Tregs were then isolated using fluorescence-activated cell sorting by gating on CD4+, CD25+, CD127low cells. After isolation, cells were stimulated with ImmunoCult Human CD3/CD28/CD2 T Cell Activator (StemCell, Cat #10970) per the manufacturer's protocol and expanded for 9 days. Cells were cultured in complete RPMI media, 10% FBS, 50 mM 2-mercaptoethanol and 1% pen/strep with hIL-2 at 300 U/mL at 1 million cells/mL. After expansion, Tregs were restimulated in the same way for 24 h before RNP electroporation.

### Results

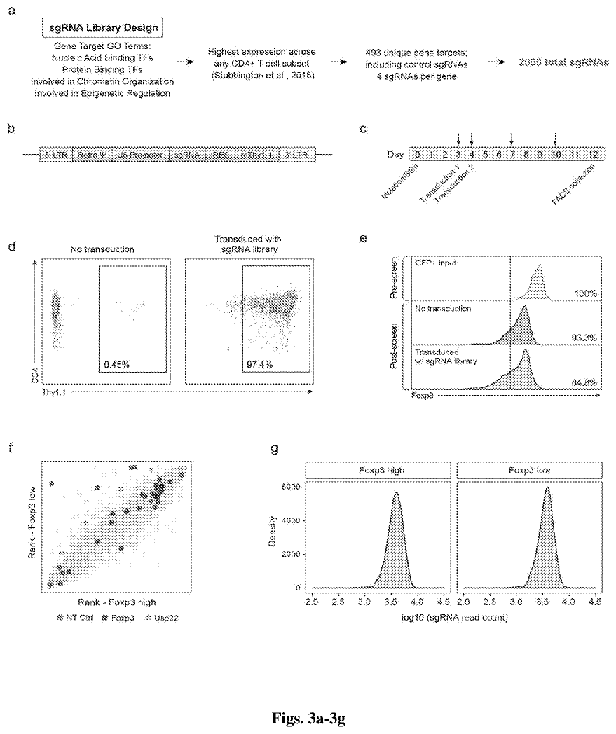

As shown in FIGS. 2a-2j and Table 1, using the methods described above, pooled CRISPR screening of transcription factors identified transcription factors that increased Foxp3 expression (Foxp3 high), including Sp1, Rnf20, Smarcb1, Satb1, Sp3 and Nsd1. As shown in FIG. 2a-j and Table 2, the screen also identified transcription factors that decreased Foxp3 expression (Foxp3 low) including, Cbfb, Myc, Atxn713, Runx1, Usp22 and Stat5b. FIGS. 3a-3g provide the design and results for the pooled CRISPR screen in primary mouse Tregs.

Additional studies were conducted to validate the role of previously undescribed candidate genes from the CRISPR screen including Rnf20 and members of the SAGA deubiquitination module, Usp22 and Atxn713. CRISPR-Cas9 ribonucleoproteins (RNP) were used to knock out candidate genes in both human and mouse primary Tregs and changes were identified in several Treg characteristic markers and pro-inflammatory cytokines by flow cytometry. Five of the top-ranking positive regulators were assessed by invidual CRISPR knockout with Cas9 RNPs. All guides tested resulted in a decrease in Foxp3 expression reproducing the screen data (FIGS. 2e and 2f).

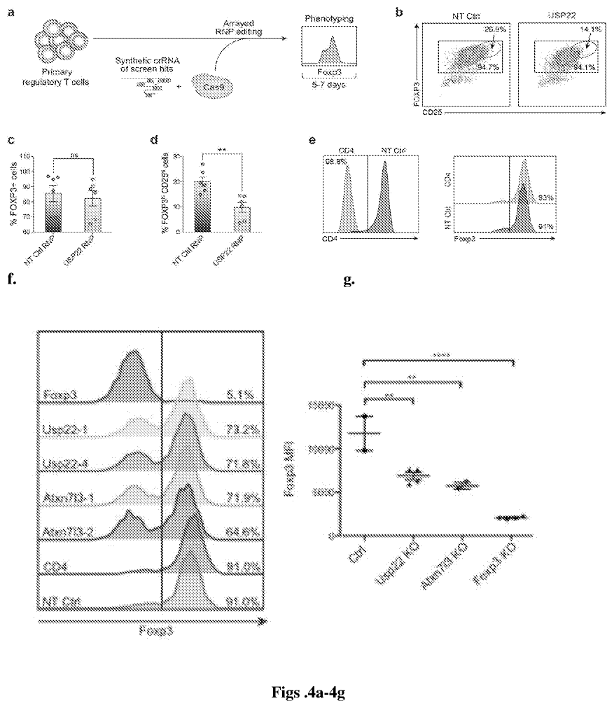

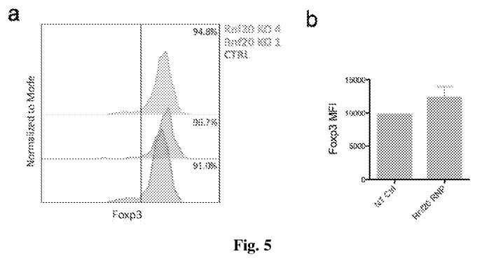

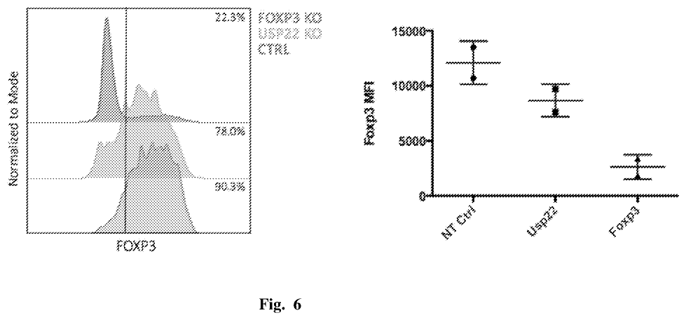

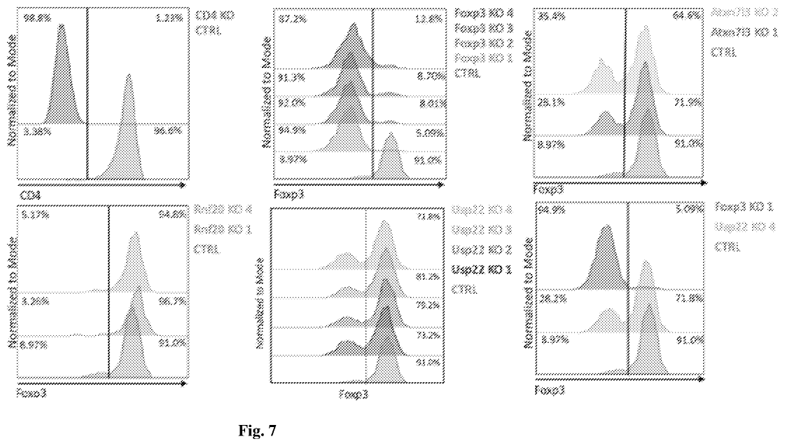

It was also found that Usp22 and Atxn713 knockouts in mouse Tregs reduces Foxp3 expression (FIGS. 4a, 4f and 4g), while Rnf20 knockdown maintains stable Foxp3 expression (FIGS. 5a, 5b and 7). FIG. 4e shows RNP controls in mouse Tregs collected 5 days post electroporation. It was also found that Usp22 knockout in human Tregs reduced Foxp3 expression (FIG. 6). Additional studies showed that knocking out USP22 with RNPs significantly decreased FOXP3 and CD25 mean fluorescence intensity (MFI) (FIGS. 2g and 2h) and frequencies of FOXP3^{hi}CD25^{hi} cells in USP22-deficient human Tregs across six biological replicates (FIGS. 4b-4d). Furthermore, quantitative assessments of genome editing were performed using sequencing based analysis tools. It was found that USP22 knockdown resulted in decreased FOXP3, CD25, and IL-10 expression, but increased IFN-γ expression compared to a scrambled non-targeting control. This data suggests that USP22 could play an important role in maintaining FOXP3 expression and Treg identity.

1. A method of increasing human regulatory T (Treg) cell stability, the method comprising:
inhibiting expression of one or more nuclear factors set forth in Table 1 and/or overexpressing one or more nuclear factors set forth in Table 2, in the human Treg cell.

2. A method of decreasing human Treg cell stability, the method comprising: inhibiting expression of a one or more nuclear factors set forth in Table 2 and/or overexpressing one or more nuclear factors set forth in Table 1, in the humanTreg cell.

3. The method of claim 1, wherein the inhibiting comprises reducing expression of the nuclear factor, or reducing expression of a polynucleotide encoding the nuclear factor.

4. The method of claim 1, wherein the overexpressing comprises increasing expression of the nuclear factor, or increasing expression of a polynucleotide encoding the nuclear factor.

5. The method of claim 4, wherein the overexpressing comprises introducing a polynucleotide encoding the nuclear factor into the Treg cell.

6. The method of claim 3, wherein the inhibiting comprises contacting a polynucleotide encoding the nuclear factor with a targeted nuclease, a guide RNA (gRNA), an siRNA, an antisense RNA, microRNA (miRNA), or short hairpin RNA (shRNA).

7. The method of claim 6, wherein the inhibiting comprises contacting the polynucleotide encoding the nuclear factor with at least one gRNA and optionally a targeted nuclease, wherein the at least one gRNA comprises a sequence selected from Table 3.

8. The method of claim 1, wherein the inhibiting comprises mutating the polynucleotide encoding the nuclear factor.

9. The method of claim 8, wherein the inhibiting comprises contacting the polynucleotide with a targeted nuclease.

10. The method of claim 9, wherein the targeted nuclease introduces a double-stranded break in a target region in the polynucleotide.

11. The method of claim 6, wherein the targeted nuclease is an RNA-guided nuclease.

12. The method of claim 11, wherein the RNA-guided nuclease is a Cpf1 nuclease or a Cas9 nuclease and the method further comprises introducing into a Treg cell a gRNA that specifically hybridizes to a target region in the polynucleotide.

13. The method of claim 12, wherein the Cpf1 nuclease or the Cas9 nuclease and the gRNA are introduced into the Treg cell as a ribonucleoprotein (RNP) complex.

14. The method of claim 9, wherein the inhibiting comprises performing clustered regularly interspaced short palindromic repeats (CRISPR)/Cas genome editing.

15. The method of claim 1, wherein the Treg cell is administered to a human following the inhibiting and/or the overexpressing.

16. The method of claim 1, wherein the Treg cell is obtained from a human prior to treating the Treg cell to inhibit expression of the nuclear factor and/or overexpress the nuclear factor, and the treated Treg cell is reintroduced into a human.

17. The method of claim 16, wherein inhibiting expression and/or overexpression results in a Treg cell having increased stability.

18. The method of claim 17, wherein the human has an autoimmune disorder.

19. The method of claim 16, wherein inhibiting expression and/or overexpression results in a Treg cell having decreased stability.

20. The method of claim 19, wherein the human has cancer.

21. A Treg cell made by the method of claim 1.

22. A Treg cell comprising:
(a) a genetic modification or heterologous polynucleotide that inhibits expression of a nuclear factor set forth in Table 1 and/or a heterologous polynucleotide that encodes a nuclear factor set forth in Table 2;
(b) a genetic modification or heterologous polynucleotide that inhibits expression of a nuclear factor set forth in Table 2 and/or a heterologous polynucleotide that encodes a nuclear factor set forth in Table 1; or
(c) at least one guide RNA (gRNA) comprising a sequence selected from Table 3.

23. (canceled)

24. (canceled)

25. The Treg cell of claim 22, wherein the expression of a nuclear factor set forth in Table 1 or Table 2 is reduced in the Treg cell relative to the expression of the nuclear factor in a Treg cell not comprising a gRNA.

26. A method of destabilizing Tregs in a subject in need thereof, comprising inhibiting expression of a one or more nuclear factors set forth in Table 2 and/or overexpressing one or more nuclear factors set forth in Table 1, in the humanTreg cells of the subject.

27. The method of claim 26, wherein inhibiting expression of a one or more nuclear factors set forth in Table 2 and/or overexpressing one or more nuclear factors set forth in Table 1 occurs in vivo.

28. The method of claim 26, wherein the method of destabilizing the Treg cells comprises:
a) obtaining Treg cells from the subject;
b) destabilizing the Treg cells by inhibiting expression of a nuclear factor set forth in Table 2 and/or overexpressing a nuclear factor set forth in Table 1 in the Treg cells; and
c) administering the destabilized Treg cells to the subject.

29. The method of claim 26, wherein the subject has cancer.

30. A method of stabilizing Tregs in a subject in need thereof, comprising inhibiting expression of a one or more nuclear factors set forth in Table 1 and/or overexpressing one or more nuclear factors set forth in Table 2, in the humanTreg cells of the subject.

31. The method of claim 30, wherein inhibiting expression of a one or more nuclear factors set forth in Table 1 and/or overexpressing one or more nuclear factors set forth in Table 2 occurs in vivo.

32. The method of claim 30, wherein the method of stabilizing the Treg cells comprises:
a) obtaining Treg cells from the subject;
b) stabilizing the Treg cells by inhibiting expression of a nuclear factor set forth in Table 1 and/or overexpressing a nuclear factor set forth in Table 2 in the Treg cells; and
c) administering the destabilized Treg cells to the subject.

33. The method of claim 30, wherein the subject has an autoimmune disorder.

34. A method of treating an autoimmune disorder in a subject, the method comprising administering a population of the Treg cells of claim 22 to a subject that has an autoimmune disease.

35. A method of treating cancer in a subject, the method comprising administering a population of the Treg cells of claim 23 to a subject that has cancer.
